SEQUENCE LISTING (237 sequences)

SEQ ID NO: 1 - protein, 42 aa, Artificial Sequence
  Human P55-TNF-R chimeric peptide
KKTAIAIAVALAGFATVAQAGSGGGGSGSSLPQIENVKGTED

SEQ ID NO: 2 - protein, 51 aa, Artificial Sequence
  Human P75-TNF-R chimeric peptide
SMAPGAVHLPQPDRVYIHPFGSSGSGGGGSTAIAIAVALAGFATVAQATKK

SEQ ID NO: 3 - protein, 43 aa, Artificial Sequence
  Human IL-6-R chimeric peptide
TSLPVQDSSSVPGSSGSGGGGSTAIAIAVALAGFATVAQATKK

SEQ ID NO: 4 - protein, 47 aa, Artificial Sequence
  L-selectin chimeric peptide
KKTAIAIAVALAGFATVAQAGSGGGGSGGGGSGSSCQKLDKSFSMIK

SEQ ID NO: 5 - protein, 50 aa, Artificial Sequence
  MUC-1 chimeric peptide
KKTAIAIAVALAGFATVAQAGSGGGGSGSSGVTSAPDTRPAPGSTAPPAH

SEQ ID NO: 6 - protein, 48 aa, Artificial Sequence
  Ovalbumin chimeric peptide I
ISQAVHAAHAEINEAGRGSSGSGGGGSTAIAIAVALAGFATVAQATKK

SEQ ID NO: 7 - protein, 38 aa, Artificial Sequence
  Ovalbumin chimeric peptide II
KKTAIAIAVALAGFATVAQAGSGGGGSGSSSIINFEKL

SEQ ID NO: 8 - protein, 51 aa, Artificial Sequence
  HIV gp120 chimeric peptide I
YNAKRKRIHIQRGPGRAFYTTKNIIGSSGSTAIAIAVALAGFATVAQATKK

SEQ ID NO: 9 - protein, 49 aa, Artificial Sequence
  HIV gp120 chimeric peptide II
KKTAIAIAVALAGFATVAQAGSGSSNNTRKSIRIQRGPGRAFVTIGKIG

SEQ ID NO: 10 - protein, 54 aa, Artificial Sequence
  HIV gp120 chimeric peptide III
KKTAIAIAVALAGFATVAQAGSGGGGSGSSCGKIEPLGVAPTKAKRRVVQREKR

SEQ ID NO: 11 - protein, 66 aa, Artificial Sequence
  HIV gp41 chimeric peptide
KKTAIAIAVALAGFATVAQAGSGGGGSGSSRVTAIEKYLQDQARLNSWGCAFRQVCHTTVPWVNDS

SEQ ID NO: 12 - protein, 34 aa, Bordetella pertussis
MNMSLSRIVKAAPLRRTTLAMALGALGAAPAAHA

SEQ ID NO: 13 - protein, 22 aa, Chlamydia trachomatis
MKKLLKSVLVFAALSSASSLQA

SEQ ID NO: 14 - protein, 22 aa, Chlamydophila psittaci
MKKLLKSALLFAATGSALSLQA

SEQ ID NO: 15 - protein, 20 aa, Escherichia coli
MIKKASLLTACSVTAFSAWA

SEQ ID NO: 16 - protein, 21 aa, Escherichia coli
MKVKVLSLLVPALLVAGAANA

SEQ ID NO: 17 - protein, 22 aa, Escherichia coli
MMKRNILAVIVPALLVAGTANA

SEQ ID NO: 18 - protein, 27 aa, Enterobacter cloacae
MKKIACLSALAAVLAVSAGTAVAATST

SEQ ID NO: 19 - protein, 24 aa, Haemophilus influenzae
MKKTNMALALLVAFSVTGCANTDI

SEQ ID NO: 20 - protein, 23 aa, Haemophilus influenzae
MNKFVKSLLVAGSVAALAACSSS

SEQ ID NO: 21 - protein, 22 aa, Haemophilus influenzae
MKKFNQSLLATAMLLAAGGANA

SEQ ID NO: 22 - protein, 20 aa, Haemophilus influenzae
MKKTLAALIVGAFAASAANA

SEQ ID NO: 23 - protein, 22 aa, Neisseria gonorrhoeae
MKKSLIALTLAALPVAAMADVT

SEQ ID NO: 24 - protein, 24 aa, Pseudomonas aeruginosa
MNNVLKFSALALAAVLATGCSSHS

SEQ ID NO: 25 - protein, 24 aa, Pseudomonas aeruginosa
MKLKNTLGVVIGSLVAASAMNAFA

SEQ ID NO: 26 - protein, 25 aa, Serratia marcescens
MNRTKLVLGAVILGSHSAGCSSNAK

SEQ ID NO: 27 - protein, 27 aa, Serratia marcescens
MILNKRLKLAYCVFLGCYGLSIHSSLA

SEQ ID NO: 28 - protein, 21 aa, Salmonella typhimurium
MKVKVLSLLVPALLVAGAANA

SEQ ID NO: 29 - protein, 20 aa, Salmonella typhimurium
MKKWLLAAGLGLAMVTSAQA

SEQ ID NO: 30 - protein, 22 aa, Escherichia coli
MNKKIHSLALLVNLGIYGVAQA

SEQ ID NO: 31 - protein, 21 aa, Cloning vector pINIIIompA3
MKKTAIAIAVALAGFATVAQA

SEQ ID NO: 32 - protein, 25 aa, Escherichia coli
MMITLRKLPLAVAVAAGVMSAQAMA

SEQ ID NO: 33 - protein, 26 aa, Escherichia coli
MKIKTGARILALSALTTMMFSASALA

SEQ ID NO: 34 - protein, 32 aa, Neisseria meningitidis
KPSLLFSSLLFSSLLFSSLLFSSLLFSSAAQA

SEQ ID NO: 35 - protein, 26 aa, Neisseria meningitidis
KNLLFSSLLFSSLLFSSLLFSSAAQA

SEQ ID NO: 36 - protein, 21 aa, Neisseria gonorrhoeae
MKAYLALISAAVIGLAACSQE

SEQ ID NO: 37 - protein, 25 aa, Haemophilus influenzae
MLNKKFKLNFIALTVAYALTPYTEA

SEQ ID NO: 38 - protein, 21 aa, Escherichia coli
MKVKVLSLLVPALLVAGAANA

SEQ ID NO: 39 - protein, 16 aa, Ralstonia solanacearum
MAAALLLWTAGTVCAA

SEQ ID NO: 40 - protein, 23 aa, Streptomyces coelicolor A3(2)
MHNSRVAVLLTTSVLTAASVGVS

SEQ ID NO: 41 - protein, 22 aa, Escherichia coli
MMKRNILAVIVPALLVAGTANA

SEQ ID NO: 42 - protein, 22 aa, Brucella melitensis biovar Abortus
MNIKSLLLGSAAALVAASGAQA

SEQ ID NO: 43 - protein, 27 aa, Neisseria gonorrhoeae
MKAKRFKINAISLSIFLAYALTPYSEA

SEQ ID NO: 44 - protein, 25 aa, Escherichia coli
MMITLRKLPLAVAVAAGVMSAQAMA

SEQ ID NO: 45 - protein, 25 aa, Haemophilus influenzae
MKKTVFRLNFLTACISLGIVSQAWA

SEQ ID NO: 46 - protein, 25 aa, Haemophilus influenzae
MKKTVFRLNFLTACVSLGIASQAWA

SEQ ID NO: 47 - protein, 22 aa, Escherichia coli
MMKRNILAVIVPALLVAGTANA

SEQ ID NO: 48 - protein, 21 aa, Escherichia coli
MKVKVLSLLVPALLVAGAANA

SEQ ID NO: 49 - protein, 26 aa, Buchnera aphidicola (Acyrthosiphon pisum)
MTNRKSLAMVIPMLLAASNGVNALEV

SEQ ID NO: 50 - protein, 21 aa, Salmonella typhimurium
MKVKVLSLLVPALLVAGAANA

SEQ ID NO: 51 - protein, 23 aa, Escherichia coli
MKKSTLALVVMGIVASASVQAAE

SEQ ID NO: 52 - protein, 23 aa, Bacteriophage PA-2
MKKLTVAISAVAASVLMAMSAQA

SEQ ID NO: 53 - protein, 21 aa, Salmonella typhimurium
MKLKLVAVAVTSLLAAGVVNA

SEQ ID NO: 54 - protein, 22 aa, Salmonella enterica subsp. enterica serovar Typhi
MKRKVLALVIPALLAAGAAHAA

SEQ ID NO: 55 - protein, 22 aa, Salmonella enterica subsp. enterica serovar Typhi
MNRKVLALLVPALLVAGAANAA

SEQ ID NO: 56 - protein, 22 aa, Salmonella enterica subsp. enterica serovar Typhi
MKVKVLSLLVPALLVAGAANAA

SEQ ID NO: 57 - protein, 22 aa, Salmonella typhi
MMKRKILAAVIPALLAAATANA

SEQ ID NO: 58 - protein, 23 aa, Salmonella typhi
MNKSTLAIVVSIIASASVHAAEV

SEQ ID NO: 59 - protein, 22 aa, Escherichia coli O157H7 EDL933
MKVKVLSLLVPALLVAGAANAA

SEQ ID NO: 60 - protein, 22 aa, Yersinia pestis
MKRNILAILIPTLLVATTSHAA

SEQ ID NO: 61 - protein, 25 aa, Yersinia pestis
MITMKLRVLSFIIPALLVAGSASAA

SEQ ID NO: 62 - protein, 23 aa, Yersinia pestis
MMKRNILAVVIPALLAAGAANAA

SEQ ID NO: 63 - protein, 23 aa, Salmonella enterica subsp. enterica serovar Typhi
MMKRKILAAVIPALLAAATANAA

SEQ ID NO: 64 - protein, 21 aa, Salmonella typhi
MKRKVLALVIPALLAAGAAHA

SEQ ID NO: 65 - protein, 21 aa, Salmonella typhi
MNRKVLALLVPALLVAGAANA

SEQ ID NO: 66 - protein, 23 aa, Salmonella typhi
MKVKVLSLLVPALLVAGAANAAE

SEQ ID NO: 67 - protein, 22 aa, Klebsiella pneumoniae
MKKTAIAIAVALAGFATVAQAA

SEQ ID NO: 68 - protein, 22 aa, Salmonella typhimurium
MKKTAIAIAVALAGFATVAQAA

SEQ ID NO: 69 - protein, 22 aa, Enterobacter aerogenes
MKKTAIAIAVALAGFATVAQAA

SEQ ID NO: 70 - protein, 22 aa, Escherichia coli
MKKTAIAIAVALAGFATVAQAA

SEQ ID NO: 71 - protein, 22 aa, Shigella dysenteriae
MKKTAIAITVALAGFATVAQAA

SEQ ID NO: 72 - protein, 22 aa, Serratia marcescens
MKKTAIALAVALAGFATVAQAA

SEQ ID NO: 73 - protein, 22 aa, Pectobacterium carotovorum
MKKTAIGLAVALAGFATVAQAA

SEQ ID NO: 74 - protein, 22 aa, Yersinia pestis
MKKTAIALAVALVGFATVAQAA

SEQ ID NO: 75 - protein, 22 aa, Pasteurella multocida
MKKTAIALTIVALAVASVAQAA

SEQ ID NO: 76 - protein, 22 aa, Buchnera sp. APS
MKKRALAIAFLLASLIPSAAQA

SEQ ID NO: 77 - protein, 25 aa, Haemophilus ducreyi
MKKTLVTLAVLSATAVATAAPQADT

SEQ ID NO: 78 - protein, 22 aa, Haemophilus sp.
MKKTAITLVVAGLAAASIAQAA

SEQ ID NO: 79 - protein, 27 aa, Bacillus subtilis
MKKTAIAIAVALAGFATVAQAGIPSST

SEQ ID NO: 80 - protein, 25 aa, Haemophilus ducreyi
MKKTLVTLSVLSATSAAFAAPDANT

SEQ ID NO: 81 - protein, 22 aa, Vibrio sp.
MKKQTLALWVGLVLAGQASMAL

SEQ ID NO: 82 - protein, 22 aa, Haemophilus influenzae Rd
MKKTAIALVVAGLAAASVAQAA

SEQ ID NO: 83 - protein, 22 aa, Haemophilus influenzae
MKKTAIALVVAGLAAASVAQAA

SEQ ID NO: 84 - protein, 22 aa, Pasteurella multocida
MKKTAIALTIAALAAASVAQAA

SEQ ID NO: 85 - protein, 25 aa, Mannheimia haemolytica
MKKTLVALAVLSAAAVAQAAPQANT

SEQ ID NO: 86 - protein, 24 aa, Sinorhizobium meliloti
TAISVGVALTGMAGMAFADPWKDE

SEQ ID NO: 87 - protein, 22 aa, Actinobacillus actinomycetemcomitans
MKRTAIALAIAGLAAATVAQAA

SEQ ID NO: 88 - protein, 22 aa, Artificial Sequence
  Signal peptide disclosed in US Patent No. 5,284,768
MXKSTLLLLFLLLCLPSWNAGA

SEQ ID NO: 89 - protein, 22 aa, Artificial Sequence
  Artificial sequence for US Patent No. 5,712,114-1
MFSFVDLRLLLLLAATALLTHG

SEQ ID NO: 90 - protein, 22 aa, Artificial Sequence
  Artificial sequence for US Patent No. 5,712,114-2
MIRLGAQSLVLLTLLVAAVLRC

SEQ ID NO: 91 - DNA, 102 bp, Bordetella pertussis
atgaacatgtctctgtcacgcattgtcaaggcggcgcccctgcgccgcaccacgctggccatggcgctgggcgcgctgggcgccgccccggcggcgcatgcc

SEQ ID NO: 92 - DNA, 66 bp, Chlamydia trachomatis
atgaaaaaactcttgaaatcggtattagtgtttgccgctttgagttctgcttcctccttgcaagct

SEQ ID NO: 93 - DNA, 66 bp, Chlamydia psittaci
atgaaaaaactcttgaaatcggcattattgtttgccgctacgggttccgctctctccttacaagcc

SEQ ID NO: 94 - DNA, 60 bp, Escherichia coli
atgattaaaaaagcttccctgctgacggcgtgttccgtcacggcattttccgcttgggca

SEQ ID NO: 95 - DNA, 63 bp, Escherichia coli
atgaaagttaaagtactgtccctcctggtcccagctctgctggtagcaggcgcagcaaacgct

SEQ ID NO: 96 - DNA, 66 bp, Escherichia coli
atgatgaagcgcaatattctggcagtgatcgtccctgctctgttagtagcaggtactgcaaacgct

SEQ ID NO: 97 - DNA, 81 bp, Enterobacter cloacae
atgaaaaaaattgcatgtctttcagcactggcagctgttctggctgtttccgcaggtaccgctgtagcggcaacttctact

SEQ ID NO: 98 - DNA, 72 bp, Haemophilus influenzae
atgaaaaaaacaaatatggcattagcactgttagttgcttttagtgtaactggttgtgcaaatactgatatt

SEQ ID NO: 99 - DNA, 69 bp, Haemophilus influenzae
atgaacaaatttgttaaatcattattagttgcaggttctgtagctgcattagcagcttgtagttcatct

SEQ ID NO: 100 - DNA, 66 bp, Haemophilus influenzae
atgaaaaaatttaatcaatctctattagcaactgcaatgttgttggctgcaggtggtgcaaatgcg

SEQ ID NO: 101 - DNA, 60 bp, Haemophilus influenzae
atgaaaaaaacacttgcagcattaatcgttggtgcattcgcagcttcagcagcaaacgca

SEQ ID NO: 102 - DNA, 66 bp, Neisseria gonorrhoeae
atgaaaaaatccctgattgccctgactttggcagcccttcctgttgcagcaatggctgacgttacc

SEQ ID NO: 103 - DNA, 72 bp, Pseudomonas aeruginosa
atgaacaacgttctgaaattctctgctctggctctggctgctgttctggccaccggttgcagcagccactcc

SEQ ID NO: 104 - DNA, 72 bp, Pseudomonas aeruginosa
atgaaactgaagaacaccttaggcgttgtcatcggctcgctggttgccgcttcggcaatgaacgccttcgcc

SEQ ID NO: 105 - DNA, 75 bp, Serratia marcescens
atgaatcgtactaaactggtactgggcgcggtaatccttggttcccactctgctggctgctctagcaacgctaaa

SEQ ID NO: 106 - DNA, 81 bp, Serratia marcescens
atgatacttaataaaagattgaagttagcgtattgcgtttttctgggttgttatggcttatccattcattcttctcttgcc

SEQ ID NO: 107 - DNA, 63 bp, Salmonella typhi
atgaaagttaaagtactgtccctcctggtaccagctctgctggtggcgggcgcagcgaatgcg

SEQ ID NO: 108 - DNA, 60 bp, Salmonella typhimurium
gtgaaaaagtggttattagctgcaggtcttggtttggcgatggtaacgtccgcacaggct

SEQ ID NO: 109 - DNA, 66 bp, Escherichia coli
atgaacaagaagattcattccctggccttgttggtcaatctggggatttatggggtagcgcaggca

SEQ ID NO: 110 - DNA, 63 bp, Escherichia coli
atgaaaaagacagctatcgcgattgcagtggcactggctggtttcgctaccgtagcgcaggcc

SEQ ID NO: 111 - DNA, 75 bp, Escherichia coli
atgatgattactctgcgcaaacttcctctggcggttgccgtcgcagcgggcgtaatgtctgctcaggcaatggct

SEQ ID NO: 112 - DNA, 78 bp, Escherichia coli
atgaaaataaaaacaggtgcacgcatcctcgcattatccgcattaacgacgatgatgttttccgcctcggctctcgcc

SEQ ID NO: 113 - DNA, 98 bp, Neisseria meningitidis
aaaaaccttctcttctcttctcttctcttctcttctcttctcttctcttctcttctcttctcttctcttctcttctcttctcttccgcagcgcaggcg

SEQ ID NO: 114 - DNA, 78 bp, Neisseria meningitidis
aaaaaccttctcttctcttctcttctcttctcttctcttctcttctcttctcttctcttctcttccgcagcgcaggcg

SEQ ID NO: 115 - DNA, 48 bp, Ralstonia solanacearum
atggccgccgcgctgctgctatggacggcaggcacggtctgcgccgcg

SEQ ID NO: 116 - DNA, 69 bp, Streptomyces coelicolor
atgcacaacagccgcgtcgcagtgctcctcacgacgtccgtactgacggccgcaagcgttggcgtgagc

SEQ ID NO: 117 - DNA, 66 bp, Brucella melitensis
atgaacatcaagagccttctccttggctccgctgcagctctggttgcagcttccggcgctcaggct

SEQ ID NO: 118 - DNA, 66 bp, Salmonella enterica
atgaaaagaaaagtattggcacttgtcatcccggctctgctggctgctggcgcagcacacgccgct

SEQ ID NO: 119 - DNA, 66 bp, Salmonella enterica
atgaacagaaaagttctggcactgcttgtcccggcgttattagtggcaggcgcagcaaatgcggct

SEQ ID NO: 120 - DNA, 66 bp, Salmonella enterica
atgaaagttaaagtactgtccctcctggtaccagctctgctggtggcgggcgcagcgaatgcggct

SEQ ID NO: 121 - DNA, 66 bp, Escherichia coli
atgaaagttaaagtactgtccctcctggtcccagctctgctggtagcaggcgcagcaaacgctgct

SEQ ID NO: 122 - DNA, 66 bp, Salmonella typhimurium
atgaaaaagacagctatcgcgattgcagtggcactggctggtttcgctaccgtagcgcaggccgct

SEQ ID NO: 123 - DNA, 66 bp, Enterobacter aerogenes
atgaaaaagacagctatcgcgattgcagtggcactggctggcttcgctaccgtagcgcaggccgct

SEQ ID NO: 124 - DNA, 66 bp, Erwinia carotovora
atgaaaaaaaccgcgatcggtctggctgtcgcgctggctggtttcgctactgtggctcaagctgcg

SEQ ID NO: 125 - DNA, 66 bp, Pasteurella multocida
atgaaaaaaacagcaattgcattgactatcgttgcactagccgtggcttcagttgcacaagctgca

SEQ ID NO: 126 - DNA, 75 bp, Haemophilus ducreyi
atgaaaaaaacattagttacattagctgtattatcagcaacagctgtagcgactgctgcgccacaagcggatact

SEQ ID NO: 127 - DNA, 66 bp, Haemophilus sp.
atgaaaaaaactgcaatcacattagtagttgctggtttagcagcagcttcaatagctcaagcagct

SEQ ID NO: 128 - DNA, 75 bp, Haemophilus ducreyi
atgaaaaaaacattagtcacattatccgtattatctgctacaagcgcagcatttgctgcaccagatgccaatacc

SEQ ID NO: 129 - DNA, 84 bp, Vibrio sp.
atgaaaaaacagatgagaatgaaaaaacagactttagccctgtgggtagggcttgtgctggcgggtcaggcatcaatggcgctg

SEQ ID NO: 130 - DNA, 66 bp, Haemophilus influenzae
atgaaaaaaactgcaatcgcattagtagttgctggcttagcagcagcttcagtagctcaagcagct

SEQ ID NO: 131 - DNA, 75 bp, Pasteurella haemolytica
atgaaaaaaacattagttgcattagcagtattatcagcagctgcagtagctcaagcagctccacaagctaacact

SEQ ID NO: 132 - DNA, 66 bp, Actinobacillus actinomycetemcomitans
atgaaaagaactgcaatcgcattagctatcgctggtttagcagcagcaacagtagcacaggcagca

SEQ ID NO: 133 - protein, 36 aa, Artificial Sequence
  Portable N-terminal SCE
MKKTAIAIAVALAGFATVAQAGGGGSGGGGSGGGGS

SEQ ID NO: 134 - protein, 36 aa, Artificial Sequence
  Portable C-terminal SCE
GSSGSGGGGSGGGGSTAIAIAVALAGFATVAQATKK

SEQ ID NO: 135 - DNA, 63 bp, Artificial Sequence; identical to SEQ ID NO: 110
  Nucleic acid sequence encoding N-SCE

SEQ ID NO: 136 - protein, 21 aa, Artificial Sequence
  Nucleic acid sequence encoding N-SCE
MKKTAIAIAVALAGFATVAQA

SEQ ID NO: 137 - DNA, 63 bp, Artificial Sequence
  Nucleic acid sequence encoding SCE-C
accgctatcgcgattgcagtggcactggctggtttcgctaccgtagcgcaggccacaaagaaa

SEQ ID NO: 138 - protein, 21 aa, Artificial Sequence
  Nucleic acid sequence encoding SCE-C
TAIAIAVALAGFATVAQATKK

SEQ ID NO: 139 - DNA, 420 bp, Mouse
  CDS (1)..(420)
tggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaa

SEQ ID NO: 140 - protein, 140 aa, Mouse
WLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 141 - DNA, 429 bp, Human
  CDS (1)..(429)
tggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggag

SEQ ID NO: 142 - protein, 143 aa, Human
WLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 143 - DNA, 543 bp, Mouse
  CDS (1)..(543)
aacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaac

SEQ ID NO: 144 - protein, 181 aa, Mouse
NNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 145 - DNA, 558 bp, Human
  CDS (1)..(558)
accaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatattgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagattttaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaac

SEQ ID NO: 146 - protein, 186 aa, Human
TNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 147 - DNA, 531 bp, Mouse
  CDS (1)..(531)
gaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaa

SEQ ID NO: 148 - protein, 177 aa, Mouse
EICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 149 - DNA, 528 bp, Human
  CDS (1)..(528)
gaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 150 - protein, 176 aa, Human
EICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 151 - DNA, 504 bp, Mouse
  CDS (1)..(504)
tacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 152 - protein, 168 aa, Mouse
YSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 153 - DNA, 396 bp, Human
  CDS (1)..(396)
cctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctgaaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcaaagcatcatctcaacactgact

SEQ ID NO: 154 - protein, 132 aa, Human
PTSSSTKKTQLQLEHLLLDLQMILNGINNYKNPKLTRMLTFKFYMPKKATELKHLQCLEEELKPLKEVLNLAQSKNFHLRPRDLISNINVIVLELKGSETTFMCEYADETATIVEFLNRWITFSQSIISTLT

SEQ ID NO: 155 - DNA, 834 bp, Mouse
  CDS (1)..(834)
cagcagcccatgaattacccatgtccccagatcttctgggtagacagcagtgccacttcatcttgggctcctccagggtcagtttttccctgtccatcttgtgggcctagagggccggaccaaaggagaccgccacctccaccaccacctgtgtcaccactaccaccgccatcacaaccactcccactgccgccactgacccctctaaagaagaaggaccacaacacaaatctgtggctaccggtggtatttttcatggttctggtggctctggttggaatgggattaggaatgtatcagctcttccacctgcagaaggaactggcagaactccgtgagttcaccaaccaaagccttaaagtatcatcttttgaaaagcaaatagccaaccccagtacaccctctgaaaaaaaagagccgaggagtgtggcccatttaacagggaacccccactcaaggtccatccctctggaatgggaagacacatatggaaccgctctgatctctggagtgaagtataagaaaggtggccttgtgatcaacgaaactgggttgtacttcgtgtattccaaagtatacttccggggtcagtcttgcaacaaccagcccctaaaccacaaggtctatatgaggaactctaagtatcctgaggatctggtgctaatggaggagaagaggttgaactactgcactactggacagatatgggcccacagcagctacctgggggcagtattcaatcttaccagtgctgaccatttatatgtcaacatatctcaactctctctgatcaattttgaggaatctaagacctttttcggcttgtataagctt

SEQ ID NO: 156 - protein, 278 aa, Mouse
QQPMNYPCPQIFWVDSSATSSWAPPGSVFPCPSCGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKEPRSVAHLTGNPHSRSIPLEWEDTYGTALISGVKYKKGGLVINETGLYFVYSKVYFRGQSCNNQPLNHKVYMRNSKYPEDLVLMEEKRLNYCTTGQIWAHSSYLGAVFNLTSADHLYVNISQLSLINFEESKTFFGLYKL

SEQ ID NO: 157 - DNA, 840 bp, Human
  CDS (1)..(840)
cagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctc

SEQ ID NO: 158 - protein, 280 aa, Human
QQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 159 - DNA, 438 bp, Chicken
  CDS (1)..(438)
aggtctttgctaatcttggtgctttgcttcctgcccctggctgctctggggaaagtctttggacgatgtgagctggcagcggctatgaagcgtcacggacttgataactatcggggatacagcctgggaaactgggtgtgtgttgcaaaattcgagagtaacttcaacacccaggctacaaaccgtaacaccgatgggagtaccgactacggaatcctacagatcaacagccgctggtggtgcaacgatggcaggaccccaggctccaggaacctgtgcaacatcccgtgctcagccctgctgagctcagacataacagcgagcgtgaactgcgcgaagaagatcgtcagcgatggaaacggcatgagcgcgtgggtcgcctggcgcaaccgctgcaagggtaccgacgtccaggcgtggatcagaggctgccggctg

SEQ ID NO: 160 - protein, 146 aa, Chicken
RSLLILVLCFLPLAALGKVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 161 - DNA, 24 bp, Artificial Sequence
  Flag tag
gactacaaggacgatgacgacaag

SEQ ID NO: 162 - protein, 8 aa, Artificial Sequence
  Flag tag
DYKDDDDK

SEQ ID NO: 163 - DNA, 18 bp, Artificial Sequence
  His tag
catcatcatcatcatcat

SEQ ID NO: 164 - protein, 6 aa, Artificial Sequence
  His tag
HHHHHH

SEQ ID NO: 165 - DNA, 27 bp, Artificial Sequence
  Strep tag
gcttggcgtcacccgcagttcggtggt

SEQ ID NO: 166 - protein, 9 aa, Artificial Sequence
  Strep tag
AWRHPQFGG

SEQ ID NO: 167 - protein, 10 aa, Artificial Sequence
  Spacer 1
GSGGGGSGSS

SEQ ID NO: 168 - protein, 3 aa, Artificial Sequence
  Spacer 2
GSS

SEQ ID NO: 169 - protein, 10 aa, Artificial Sequence
  Spacer 3
GSSGSGGGGS

SEQ ID NO: 170 - DNA, 15 bp, Artificial Sequence
  Spacer 1, n = 0
ggatccggctcgagt

SEQ ID NO: 171 - protein, 5 aa, Artificial Sequence
  Spacer 1, n = 0
GSGSS

SEQ ID NO: 172 - DNA, 30 bp, Artificial Sequence
  Spacer 1, n = 1
ggatccggtggtggtggatccggctcgagt

SEQ ID NO: 173 - protein, 10 aa, Artificial Sequence
  Spacer 1, n = 1
GSGGGGSGSS

SEQ ID NO: 174 - DNA, 45 bp, Artificial Sequence
  Spacer 1, n = 2
ggatccggtggtggtggtagcggtggtggtggatccggctcgagt

SEQ ID NO: 175 - protein, 15 aa, Artificial Sequence
  Spacer 1, n = 2
GSGGGGSGGGGSGSS

SEQ ID NO: 176 - DNA, 45 bp, Artificial Sequence; identical to SEQ ID NO: 174
  Spacer 1

SEQ ID NO: 177 - DNA, 9 bp, Artificial Sequence
  Spacer 2
ggctcgagt

SEQ ID NO: 178 - DNA, 15 bp, Artificial Sequence
  Spacer 3, n=0
ggctcgagtggatcc

SEQ ID NO: 179 - protein, 5 aa, Artificial Sequence
  Spacer 3, n = 0
GSSGS

SEQ ID NO: 180 - DNA, 30 bp, Artificial Sequence
  Spacer 3, n = 1
ggctcgagtggatccggtggtggtggatcc

SEQ ID NO: 181 - protein, 10 aa, Artificial Sequence
  Spacer 3, n = 1
GSSGSGGGGS

SEQ ID NO: 182 - DNA, 45 bp, Artificial Sequence
  Spacer 3, n = 2
ggctcgagtggatccggtggtggtggtagcggtggtggtggatcc

SEQ ID NO: 183 - protein, 15 aa, Artificial Sequence
  Spacer 3, n = 2
GSSGSGGGGSGGGGS

SEQ ID NO: 184 - DNA, 45 bp, Artificial Sequence; identical to SEQ ID NO: 182
  Spacer 3

SEQ ID NO: 185 - DNA, 552 bp, Artificial Sequence
  Murine GM-CSF chimeric construct
atgaaaaagacagctatcgcgattgcagtggcactggctggtttcgctaccgtagcgcaggccggatccggtggtggtggatccggctcgagttggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaaggctcgagtgactacaaggacgatgacgacaagtaataa

SEQ ID NO: 186 - protein, 182 aa, Artificial Sequence
  Murine GM-CSF chimeric construct
MKKTAIAIAVALAGFATVAQAGSGGGGSGSSWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQKGSSDYKDDDDK

SEQ ID NO: 187 - DNA, 579 bp, Artificial Sequence
  Human GM-CSF chimeric construct
atggactacaaggacgatgacgacaagggctcgagttggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggctcgagtggatccggtggtggtggtagcggtggtggtggatccaccgctatcgcgattgcagtggcactggctggtttcgctaccgtagcgcaggccacaaagaaataataa

SEQ ID NO: 188 - protein, 191 aa, Artificial Sequence
  Human GM-CSF chimeric construct
MDYKDDDDKGSSWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGSSGSGGGGSGGGGSTAIAIAVALAGFATVAQATKK

SEQ ID NO: 189 - DNA, 732 bp, Artificial Sequence
  Murine IFN-beta chimeric construct
atgcatcatcatcatcatcatggctcgagtaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaacggctcgagtggatccggtggtggtggtagcggtggtggtggtagcggtggtggtggtagcggtggtggtggtagcggtggtggtggatccaccgctatcgcgattgcagtggcactggctggtttcgctaccgtagcgcaggccacaaagaaataataa

SEQ ID NO: 190 - protein, 242 aa, Artificial Sequence
  Murine IFN-beta chimeric construct
MHHHHHHGSSNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQNGSSGSGGGGSGGGGSGGGGSGGGGSGGGGSTAIAIAVALAGFATVAQATKK

SEQ ID NO: 191 - DNA, 708 bp, Artificial Sequence
  Human IFN-beta chimeric construct
atgaaaaagacagctatcgcgattgcagtggcactggctggtttcgctaccgtagcgcaggccggatccggtggtggtggtagcggtggtggtggatccggctcgagtaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatattgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagattttaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaacggctcgagtgcttggcgtcacccgcagttcggtggttaataa

SEQ ID NO: 192 - protein, 234 aa, Artificial Sequence
  Human IFN-beta chimeric construct
MKKTAIAIAVALAGFATVAQAGSGGGGSGGGGSGSSTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGSSAWRHPQFGG

SEQ ID NO: 193 - DNA, 723 bp, Artificial Sequence
  Murine IL-1Ra chimeric construct
atgaaaaagacagctatcgcgattgcagtggcactggctggtttcgctaccgtagcgcaggccggatccggtggtggtggtagcggtggtggtggtagcggtggtggtggtagcggtggtggtggtagcggtggtggtggatccggctcgagtgaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaaggctcgagtgactacaaggacgatgacgacaagtaataa

SEQ ID NO: 194 - protein, 239 aa, Artificial Sequence
  Murine IL-1Ra chimeric construct
MKKTAIAIAVALAGFATVAQAGSGGGGSGGGGSGGGGSGGGGSGGGGSGSSEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQGSSDYKDDDDK

SEQ ID NO: 195 - DNA, 642 bp, Artificial Sequence
  Human IL-1Ra chimeric construct
atgcatcatcatcatcatcatggctcgagtgaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagggctcgagtggatccaccgctatcgcgattgcagtggcactggctggtttcgctaccgtagcgcaggccacaaagaaataataa

SEQ ID NO: 196 - protein, 212 aa, Artificial Sequence
  Human IL-1Ra chimeric construct
MHHHHHHGSSEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEGSSGSTAIAIAVALAGFATVAQATKK

SEQ ID NO: 197 - DNA, 642 bp, Artificial Sequence
  Murine IL-2 chimeric construct
atggcttggcgtcacccgcagttcggtggtggctcgagttacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggctcgagtggatccggtggtggtggatccaccgctatcgcgattgcagtggcactggctggtttcgctaccgtagcgcaggccacaaagaaataataa

SEQ ID NO: 198 - protein, 212 aa, Artificial Sequence
  Murine IL-2 chimeric construct
MAWRHPQFGGGSSYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGSSGSGGGGSTAIAIAVALAGFATVAQATKK

SEQ ID NO: 199 - DNA, 513 bp, Artificial Sequence
  Human IL-2 chimeric construct
atgaaaaagacagctatcgcgattgcagtggcactggctggtttcgctaccgtagcgcaggccggatccggctcgagtcctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctgaaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcaaagcatcatctcaacactgactggctcgagtgactacaaggacgatgacgacaagtaataa

SEQ ID NO: 200 - protein, 169 aa, Artificial Sequence [sequence omitted]

SEQ ID NO: 201 - DNA, 960 bp, Artificial Sequence
  Murine Fas-L chimeric construct
atgaaaaagacagctatcgcgattgcagtggcactggctggtttcgctaccgtagcgcaggccggatccggtggtggtggatccggctcgagtcagcagcccatgaattacccatgtccccagatcttctgggtagacagcagtgccacttcatcttgggctcctccagggtcagtttttccctgtccatcttgtgggcctagagggccggaccaaaggagaccgccacctccaccaccacctgtgtcaccactaccaccgccatcacaaccactcccactgccgccactgacccctctaaagaagaaggaccacaacacaaatctgtggctaccggtggtatttttcatggttctggtggctctggttggaatgggattaggaatgtatcagctcttccacctgcagaaggaactggcagaactccgtgagttcaccaaccaaagccttaaagtatcatcttttgaaaagcaaatagccaaccccagtacaccctctgaaaaaaaagagccgaggagtgtggcccatttaacagggaacccccactcaaggtccatccctctggaatgggaagacacatatggaaccgctctgatctctggagtgaagtataagaaaggtggccttgtgatcaacgaaactgggttgtacttcgtgtattccaaagtatacttccggggtcagtcttgcaacaaccagcccctaaaccacaaggtctatatgaggaactctaagtatcctgaggatctggtgctaatggaggagaagaggttgaactactgcactactggacagatatgggcccacagcagctacctgggggcagtattcaatcttaccagtgctgaccatttatatgtcaacatatctcaactctctctgatcaattttgaggaatctaagacctttttcggcttgtataagcttggctcgagtcatcatcatcatcatcattaataa

SEQ ID NO: 202 - protein, 318 aa, Artificial Sequence
  Murine Fas-L chimeric construct
MKKTAIAIAVALAGFATVAQAGSGGGGSGSSQQPMNYPCPQIFWVDSSATSSWAPPGSVFPCPSCGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKEPRSVAHLTGNPHSRSIPLEWEDTYGTALISGVKYKKGGLVINETGLYFVYSKVYFRGQSCNNQPLNHKVYMRNSKYPEDLVLMEEKRLNYCTTGQIWAHSSYLGAVFNLTSADHLYVNISQLSLINFEESKTFFGLYKLGSSHHHHHH

SEQ ID NO: 203 - DNA, 993 bp, Artificial Sequence
  Human Fas-L chimeric construct
atggcttggcgtcacccgcagttcggtggtggctcgagtcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctcggctcgagtggatccggtggtggtggtagcggtggtggtggatccaccgctatcgcgattgcagtggcactggctggtttcgctaccgtagcgcaggccacaaagaaataataa

SEQ ID NO: 204 - protein, 329 aa, Artificial Sequence
  Human Fas-L chimeric construct
MAWRHPQFGGGSSQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKLGSSGSGGGGSGGGGSTAIAIAVALAGFATVAQATKK

SEQ ID NO: 205 - DNA, 633 bp, Artificial Sequence
  HEL chimeric construct
atggactacaaggacgatgacgacaagggctcgagtaggtctttgctaatcttggtgctttgcttcctgcccctggctgctctggggaaagtctttggacgatgtgagctggcagcggctatgaagcgtcacggacttgataactatcggggatacagcctgggaaactgggtgtgtgttgcaaaattcgagagtaacttcaacacccaggctacaaaccgtaacaccgatgggagtaccgactacggaatcctacagatcaacagccgctggtggtgcaacgatggcaggaccccaggctccaggaacctgtgcaacatcccgtgctcagccctgctgagctcagacataacagcgagcgtgaactgcgcgaagaagatcgtcagcgatggaaacggcatgagcgcgtgggtcgcctggcgcaaccgctgcaagggtaccgacgtccaggcgtggatcagaggctgccggctgggctcgagtggatccggtggtggtggtagcggtggtggtggtagcggtggtggtggtagcggtggtggtggtagcggtggtggtggatccaccgctatcgcgattgcagtggcactggctggtttcgctaccgtagcgcaggccacaaagaaataataa

SEQ ID NO: 206 - protein, 209 aa, Artificial Sequence
  HEL chimeric construct
MDYKDDDDKGSSRSLLILVLCFLPLAALGKVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRLGSSGSGGGGSGGGGSGGGGSGGGGSGGGGSTAIAIAVALAGFATVAQATKK

SEQ ID NO: 207 - DNA, 282 bp, Mouse
  CDS (1)..(282)
aagatttccacacttctatgcctcctgctcatagctaccaccatcagtcctcaggtattggctggaccagatgcggtgagcaccccagtcacgtgctgttataatgttgttaagcagaagattcacgtccggaagctgaagagctacaggagaatcacaagcagccagtgtccccgggaagctgtgatcttcaggaccatactggataaggagatctgtgctgaccccaaggagaagtgggttaagaattccataaaccacttggataagacgtctcgaacg

SEQ ID NO: 208 - protein, 94 aa, Mouse
KISTLLCLLLIATTISPQVLAGPDAVSTPVTCCYNVVKQKIHVRKLKSYRRITSSQCPREAVIFRTILDKEICADPKEKWVKNSINHLDKTSRT

SEQ ID NO: 209 - DNA, 294 bp, Human
  CDS (1)..(294)
aaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagact

SEQ ID NO: 210 - protein, 98 aa, Human
KVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 211 - DNA, 402 bp, Artificial Sequence
  Murine MCP-1 chimeric construct
atgaaaaagacagctatcgcgattgcagtggcactggctggtttcgctaccgtagcgcaggccggatccggctcgagtaagatttccacacttctatgcctcctgctcatagctaccaccatcagtcctcaggtattggctggaccagatgcggtgagcaccccagtcacgtgctgttataatgttgttaagcagaagattcacgtccggaagctgaagagctacaggagaatcacaagcagccagtgtccccgggaagctgtgatcttcaggaccatactggataaggagatctgtgctgaccccaaggagaagtgggttaagaattccataaaccacttggataagacgtctcgaacgggctcgagtgcttggcgtcacccgcagttcggtggttaataa

SEQ ID NO: 212 - protein, 132 aa, Artificial Sequence
  Murine MCP-1 chimeric construct
MKKTAIAIAVALAGFATVAQAGSGSSKISTLLCLLLIATTISPQVLAGPDAVSTPVTCCYNVVKQKIHVRKLKSYRRITSSQCPREAVIFRTILDKEICADPKEKWVKNSINHLDKTSRTGSSAWRHPQFGG

SEQ ID NO: 213 - DNA, 405 bp, Artificial Sequence
  Human MCP-1 chimeric construct
atgaaaaagacagctatcgcgattgcagtggcactggctggtttcgctaccgtagcgcaggccggatccggctcgagtaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagactggctcgagtcatcatcatcatcatcattaataa

SEQ ID NO: 214 - protein, 133 aa, Artificial Sequence
  Human MCP-1 chimeric construct
MKKTAIAIAVALAGFATVAQAGSGSSKVSAALLCLLLIAATFIPQGLAQPDAINAPVTCCYNFTNRKISVQRLASYRRITSSKCPKEAVIFKTIVAKEICADPKQKWVQDSMDHLDKQTQTPKTGSSHHHHHH

SEQ ID NO: 215 - protein, 20 aa, Artificial Sequence
  N-SCE2
KKTAIAIAVALAGFATVAQA

SEQ ID NO: 216 - protein, 69 aa, Artificial Sequence
  Human ACTH chimeric peptide
KKTAIAIAVALAGFATVAQAGSGGGGSGSSSYSMEHFRWGKPVGKKRRPVKVYPNGAEDESAEAFPLEF

SEQ ID NO: 217 - protein, 65 aa, Artificial Sequence
  Murine ACTH chimeric peptide
SYSMEHFRWGKPVGKKRRPVKVYPNVAENESAEAFPLEFGSSGSTAIAIAVALAGFATVAQATKK

SEQ ID NO: 218 - protein, 44 aa, Artificial Sequence
  Human alpha MSH chimeric peptide
SYSMEHFRWGKPVGSSGSGGGGSTAIAIAVALAGFATVAQATKK

SEQ ID NO: 219 - protein, 47 aa, Artificial Sequence
  Human beta MSH chimeric peptide
KKTAIAIAVALAGFATVAQAGSGSSAEKKDEGPYRMEHFRWGSPPKD

SEQ ID NO: 220 - protein, 52 aa, Artificial Sequence
  Murine beta MSH chimeric peptide
AEKDDGPYRVEHFRWSNPPKDGSSGSGGGGSTAIAIAVALAGFATVAQATKK

SEQ ID NO: 221 - protein, 37 aa, Artificial Sequence
  Human gamma MSH chimeric peptide
KKTAIAIAVALAGFATVAQAGSGSSYVMGHFRWDRFG

SEQ ID NO: 222 - protein, 40 aa, Artificial Sequence
  Human angiotensin I chimeric peptide
KKTAIAIAVALAGFATVAQAGSGGGGSGSSDRVYIHPFHL

SEQ ID NO: 223 - protein, 39 aa, Artificial Sequence
  Human angiotensin II chimeric peptide
DRVYIHPFGSSGSGGGGSTAIAIAVALAGFATVAQATKK

SEQ ID NO: 224 - protein, 37 aa, Artificial Sequence
  Human angiotensin III chimeric peptide
KKTAIAIAVALAGFATVAQAGSGGGGSGSSRVYIHPF

SEQ ID NO: 225 - protein, 55 aa, Artificial Sequence
  Human GHRH chimeric peptide I
YFDAIFTNSYRKVLGQLSARKLLQDIMSRGSSGSTAIAIAVALAGFATVAQATKK

SEQ ID NO: 226 - protein, 70 aa, Artificial Sequence
  Human GHRH chimeric peptide II
YADAIFTNSYRKVLGQLSARKLLQDIMSRQQGESNQERGARARLGSSGSTAIAIAVALAGFATVAQATKK

SEQ ID NO: 227 - protein, 67 aa, Artificial Sequence
  Murine GHRH chimeric peptide
KKTAIAIAVALAGFATVAQAGSGSSHVDAIFTTNYRKLLSQLYARKVIQDIMNKQGERIQEQRARLS

SEQ ID NO: 228 - protein, 35 aa, Artificial Sequence
  Human IL-1 beta chimeric peptide I
VQGEESNDKGSSGSTAIAIAVALAGFATVAQATKK

SEQ ID NO: 229 - protein, 60 aa, Artificial Sequence
  Human IL-1 beta chimeric peptide II
KKTAIAIAVALAGFATVAQAGSGGGGSGSSLKEKNLYLSCVLKDDKPTLQLESVDPKNYP

SEQ ID NO: 230 - protein, 38 aa, Artificial Sequence
  Human IL-2 chimeric peptide I
KKTAIAIAVALAGFATVAQAGSGSSEYADETATIVEFL

SEQ ID NO: 231 - protein, 44 aa, Artificial Sequence
  Human IL-2 chimeric peptide II
ILNGINNYKNPKLGSSGSGGGGSTAIAIAVALAGFATVAQATKK

SEQ ID NO: 232 - protein, 41 aa, Artificial Sequence
  Human IL-2 chimeric peptide III
KKTAIAIAVALAGFATVAQAGSGGGGSGSSLTFKFYMPKKA

SEQ ID NO: 233 - protein, 43 aa, Artificial Sequence
  Human TNF-alpha chimeric peptide I
SPLAQAVRSSSRGSSGSGGGGSTAIAIAVALAGFATVAQATKK

SEQ ID NO: 234 - protein, 52 aa, Artificial Sequence
  Human TNF-alpha chimeric peptide II
KKTAIAIAVALAGFATVAQAGSGSSDKPVAHVVANPQAEGQLQWLNRRANAL

SEQ ID NO: 235 - protein, 46 aa, Artificial Sequence
  Human TNF-alpha chimeric peptide III
RRANALLANGVELRDGSSGSGGGGSTAIAIAVALAGFATVAQATKK

SEQ ID NO: 236 - protein, 54 aa, Artificial Sequence
  Human Cys-BAFF-R chimeric peptide I
CLRGASSAEAPDGDKDAPEPLDKGSSGSGGGGSTAIAIAVALAGFATVAQATKK

SEQ ID NO: 237 - protein, 56 aa, Artificial Sequence
  Human Cys-BAFF-R chimeric peptide II
KKTAIAIAVALAGFATVAQAGSGGGGSGSSCHSVPVPATELGSTELVTTKTAGPEQ

(19) United States
(12) Patent Application Publication
(10) Pub. No.: US 2004/0029179 A1
(43) Pub. Date: Feb. 12, 2004

(54) Higher molecular weight entities and uses therefor

(76) Inventor: Frank Koentgen, North Beach (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/449,831

(22) Filed: May 30, 2003

Related U.S. Application Data
(60) Provisional application No. 60/384,878, filed on May 31, 2002.

(51) Int. Cl. G01N 33/53; C07K 14/435; C07K 14/195; C07K 14/005

(52) U.S. Cl. 435/7.1; 530/350

(57) ABSTRACT

The present invention discloses a method for enhancing the activity of a molecule, or for combining individual activities of different molecules, by linking, fusing or otherwise associating the molecule(s) with a self-coalescing element, whereby the chimeric molecule so formed self-assembles into a higher molecular weight aggregate. The present invention also discloses such chimeric molecules per se and to their use in therapeutic, prophylactic and chemical process applications.

### FIELD OF THE INVENTION

THIS INVENTION relates generally to active molecules and more particularly to a method for enhancing the activity of a molecule, or for combining individual activities of different molecules, by linking, fusing or otherwise associating the molecule(s) with a self-coalescing element, whereby the chimeric molecule so formed self-assembles into a higher molecular weight aggregate. The present invention also relates to those chimeric molecules per se and to their use in therapeutic, prophylactic and chemical process applications.

### BACKGROUND OF THE INVENTION

There is much interest in using biochemical or molecular biological techniques to produce proteins with novel or enhanced properties. One desirable property is enhancing the biological activity of a protein such as increasing its circulating half-life or immunogenicity.

Several methods have been employed to enhance the biological activity of proteins and these often focus on increasing the size of the molecules. One method of increasing a protein's size is through chemical cross-linking with another protein. For example, to increase the immunogenicity of a protein, chemical cross-linking agents are used to conjugate an antigen of interest to a carrier protein. The carrier serves to non-specifically stimulate T helper cell activity and to direct the antigen to an antigen-presenting cell (e.g., a professional antigen-presenting cell such as a dendritic cell), where the antigen is processed and presented at the cell surface in the context of the major histocompatibility complex (MHC).

Several carrier systems have been developed for this purpose. For example, small peptide antigens are often coupled to protein carriers such as tetanus toxoid (Muller et al., 1982, Proc. Natl. Acad. Sci. U.S.A. 79: 569-573), keyhole limpet haemocyanin (Bittle et al., 1982, Nature 298: 30-33), ovalbumin, and sperm whale myoglobin, to raise an immune response. However, carriers may elicit strong immunity not relevant to the peptide antigen and this may inhibit the immune response to the peptide vaccine on secondary immunisation (Schutze et al., J. Immunol. 135: 2319-2322).

Antigen delivery systems have also been based on particulate carriers. For example, preformed particles have been used as platforms onto which antigens can be coupled and incorporated. Systems based on proteosomes (Lowell et al., 1988, Science 240: 800-802), immune stimulatory complexes (Morein 1984, Nature 308: 457460), and viral particles such as HBsAg (Neurath et al., 1989, Mol. Immunol. 26: 53-62) and rotavirus inner capsid protein (Redmond et al., 1991, Mol. Immunol. 28: 269-278) have been developed.

Other carrier systems have been devised using recombinantly produced chimeric viral capsid proteins or viral core proteins that self assemble into virus-like particles (VLP) or viral core-like particles (CLP), respectively. Representative chimeric particles of this type include those based on yeast Ty protein (Kingsman and Kirigsman 1988, Vacc. 6: 304-306), HBsAg, (Valenzuela, 1985, Bio/Technol. 3: 323-326; U.S. Pat. No. 4,722,840; Delpeyroux et al., 1986, Science 233: 472-475), Hepatitis B core antigen (Clarke et al., Vaccines 88 (Ed. H. Ginsberg, et al., 1988) pp. 127-131), the capsid protein from Poliovirus (Burke et al., 1988, Nature 332: 81-82) or Parvovirus (Brown et al., 1994, Virology 198: 477488), and the L1 and L2 capsid proteins of papillomavirus (U.S. Pat. No. 5,618,536). However, these carriers are restricted in their usefulness by virtue of the limited size of the antigen that may be inserted into the structural protein without interfering with particle assembly.

Alternatives, such as peptide linkers have been used to enhance the combined biological activities of two or more different proteins. For example, U.S. Pat. No. 5,073,627 describes the use of a peptide linker to join a Granulocyte-Macrophage Colony-Stimulating Factor (GM-CSF) protein molecule to an Interleukin-3 (IL-3) protein molecule to form a fusion protein, which was more biologically active than GM-CSF or IL-3 alone or in combination. Conventional peptide linkers, however, can be rigid and inflexible. As a result, the linked protein often cannot “flex” into the desired biologically active conformation exhibited by the wild type protein, or the cross-linker or carrier protein sterically hinders biological activity.

Signal peptide sequences (also known as leader sequences) are membrane translocating sequences, which mediate secretion of proteins into various intracellular compartments or the extracellular environment. Typically, signal sequences comprise about 15 to 35 residues and are composed of a positively charged. amino terminus, a central hydrophobic region and a short chain amino acid at the carboxyl terminus. Signal sequences used for targeting proteins to specific locations have been found in both prokaryotic and eukaryotic cells. In bacteria, phage fd signal sequences for the major and minor coat proteins direct those proteins to the inner membrane. The β-lactamase protein of pBR322 is directed to the periplasmic space by a different signal sequence, while outer membrane proteins such as OmpA are directed to their assigned destination by other signal sequences. Eukaryotic signal sequences directing translocation of the protein into the endoplasmic reticulum include that of human preproinsulin, bovine growth hormone, and the Drosophila glue protein. Near the N-terminus of such sequences are 2-3 polar residues, and within the signal sequence is a hydrophobic core consisting of hydrophobic amino acids. No other conservation of sequence has been observed (Lewin, B.,1994, Genes V, Oxford University Press, p. 290; Watson, M., 1984, Nucl. Acids. Res. 12:5145-5164).

Biological membrane transport has been exploited for protein expression and export from transfected or transformed cells. Secretion of proteins, such as a globin protein, which would normally remain in the cytosol, has been achieved by adding a signal sequence to the N-terminus of the protein (Lewin, B., 1994, supra). Foreign genes have been inserted into recombinant DNA constructs for expression and secretion from bacterial cells, as described for example in U.S. Pat. No. 5,156,959, which discloses a method to export gene products into the growth medium of gram negative bacteria. U.S. Pat. No. 5,380,653 describes expression vectors and methods for intracellular protein production in Bacillus species. U.S. Pat. No. 5,712,114 describes a recombinant DNA construct for secretion of expressed proteins, particularly from Hansenula polymorpha cells, which utilises the signal sequence of the human preprocollagen α-1 protein.

International publication WO97/35887 describes a B cell mitogen precursor and its use for the production of antigen-specific catalytic antibodies. The precursor comprises a T cell surface molecule binding portion (H) from hen egg lysozyme (HEL), flanked by a pair of immunoglobulin-binding domains (L) from protein L of Peptostreptococcus magnus as B cell surface molecule binding portions. The specificity of the LHL construct for catalytic B cells is provided by an antigen masking the immunoglobulin-binding domains. Catalytic cleavage of the antigen exposes the immunoglobulin-binding domains to ligate the immunoglobulin molecules on the B cell surface, to thereby permit catalytic antibody production by the B cell. For recombinant production of the mitogen precursor, the OmpA signal peptide was fused with the B cell mitogen precursor as a means for targeting expression of the precursor to the periplasmic space of a bacterium. The resultant fusion protein, however, was found unexpectedly to self assemble into a higher molecular weight aggregate. The multimerising capacity of the OmpA signal peptide was exploited to design a non-specific B cell mitogen that cross-links immunoglobulin molecules on the surface of any B cell. This B cell mitogen was constructed by fusing the OmpA signal peptide to the C-terminus of an immunoglobulin-binding domain from protein L.

Fundamentally, therefore, signal sequences have been used in the context of protein expression systems. They have also been used as a means to cross-link immunoglobulin molecules on the surface of B cells. However, the use of signal sequences, generally, to enhance the biological activity (e.g., longer circulating half-life, higher potency or enhanced immunogenicity) of a molecule or to combine the individual activities of different molecules, has not heretofore been described.

### SUMMARY OF THE INVENTION

One aspect of the present invention provides methods for enhancing the activity of a molecule of interest, or for combining distinct activities of different molecules of interest. These methods generally comprise linking, fusing or otherwise associating individual molecules of interest with a self-coalescing element (SCE) that is obtainable or derivable from a membrane translocating sequence (MTS) or variant thereof. The chimeric molecule formed by this process is caused by the SCE to coalesce with other such molecules into a higher molecular weight aggregate with enhanced or improved properties relative to the non-aggregated molecules.

The molecule of interest may be selected from any compound, organic or inorganic, but is usually a polymer and typically a polypeptide having a desired biological activity, including an enzymatic, antigenic or therapeutic activity. Thus, the present invention also contemplates a chimeric polypeptide comprising an SCE as broadly described above, which is fused, linked or otherwise associated with a polypeptide of interest, and which causes an individual chimeric molecule to coalesce with other chimeric molecules into higher order aggregates under conditions favorable to aggregation. In a related aspect, the present invention extends to isolated or purified higher order aggregates comprising a plurality of such chimeric molecules.

The present invention also extends to processes for producing the chimeric molecules of the invention. In certain embodiments, the chimeric molecules are produced by chemical synthesis. In other embodiments, the chimeric molecules are produced by chemically fusing SCEs with individual molecules of interest. In still other embodiments, the chimeric molecules are produced by recombinant means and, in this regard, expression vectors and host cells, as well as methods of producing chimeric polypeptides in host cells, or in genetically modified animals, are also encompassed by the present invention.

The present invention also extends to methods of using the higher order aggregates described herein in a range of applications, including chemical, therapeutic and prophylactic applications. In one embodiment, a higher order aggregate comprises only identical, or substantially similar, molecules of interest, whereby such “homo-aggregates” are useable in the same manner as the non-aggregated parent molecules of interest, especially where an increased biological activity is desirable. For example, higher order aggregates comprising GM-CSF-SCE chimeric polypeptides, which have a higher GM-CSF potency compared to non aggregated GM-CSF, can be used to treat various haemopoetic conditions, as described infra. In another embodiment, higher order aggregates comprising two or more distinct biological activities can be used to produce a desired biological outcome resulting from the product of those activities. For example, a pair of chimeric polypeptides can be constructed, wherein a first chimeric polypeptide comprises interleukin-2 (IL-2) and wherein a second chimeric polypeptide comprises Fas ligand. Higher order aggregates comprising these chimeric polypeptides are useful in targeting certain leukemia or lymphoma cells, or recently activated T cells which bear both high affinity IL-2R and Fas. Aggregates comprising a plurality of distinct chimeric polypeptides whose collective activities are required to achieve a biological effect will generally increase the speed and/or efficiency of the process resulting in the biological effect due to the close proximity of the distinct polypeptides of interest. Thus, “hetero-aggregates”, containing two or more different polypeptides, can exhibit synergistic characteristics, and thus exhibit an activity greater than the activity that would be exhibited by a similar quantity of each polypeptide found in the hetero-aggregate if each polypeptide component were to be used alone.

### BRIEF DESCRIPTION OF THE DRAWINGS

FIG. 1 is a diagrammatic representation showing an alignment of membrane translocating amino acid sequences from a diverse selection of species.

FIG. 2 is a diagrammatic representation showing an alignment of bacterial outer membrane proteins.

### BRIEF DESCRIPTION OF THE SEQUENCES

TABLE A

| SEQUENCE ID | | | |
|---|---|---|---|
| NUMBER | SEQUENCE | LENGTH | |
| SEQ ID NO: 1 | Formula I | 14 | subunits |
| SEQ ID NO: 2 | Formula II | 3 | subunits |
| SEQ ID NO: 3 | Formula III | 5 | subunits |
| SEQ ID NO: 4 | Formula IV | 6 | subunits |
| SEQ ID NO: 5 | Formula V | 4 | subunits |
| SEQ ID NO: 6 | Formula VI | 4 | subunits |
| SEQ ID NO: 7 | Formula VII | 16 | subunits |
| SEQ ID NO: 8 | Formula VIII | 19 | subunits |
| SEQ ID NO: 9 | Formula IX | 5 | subunits |
| SEQ ID NO: 10 | Formula X | 2 | subunits |
| SEQ ID NO: 11 | Formula XI | 3 | subunits |
| SEQ ID NO: 12 | Signal peptide relating to P.69 outer membrane protein precursor - | 34 | residues |
| | Bordetella Pertussis - GenBank Accession No. AAA22980 | | |
| SEQ ID NO: 13 | Signal peptide relating to major outer membrane protein precursor - | 22 | residues |
| | Chlamydia trachomatis - GenBank Accession No. AAA23142 | | |
| SEQ ID NO: 14 | Signal peptide relating to major outer membrane protein (MOMP) | 22 | residues |
| | precursor - Chlamydophila psittaci - GenBank Accession No. | | |
| | AAA23146 | | |
| SEQ ID NO: 15 | Signal peptide relating to B12 receptor protein BtuB - Escherichia | 20 | residues |
| | coli - GenBank Accession No. AAA23524 | | |
| SEQ ID NO: 16 | Signal peptide relating to outer membrane protein - Escherichia | 21 | residues |
| | coli - GenBank No. AAA24243 | | |
| SEQ ID NO: 17 | Signal peptide relating to Pro-OmpF outer membrane protein - | 22 | residues |
| | Escherichia coli - GenBank Accession No. AAA24244 | | |
| SEQ ID NO: 18 | Signal peptide relating to outer membrane protein X precursor - | 27 | residues |
| | Enterobacter cloacae - GenBank Accession No. AAA24808 | | |
| SEQ ID NO: 19 | Signal peptide relating to 15kd peptidoglycan-associated outer | 24 | residues |
| | membrane lipoprotein precursor - Haemophilus influenzae - | | |
| | GenBank Accession No. AAA24938 | | |
| SEQ ID NO: 20 | Signal peptide relating to PC protein precursor - Haemophilus | 23 | residues |
| | influenzae - GenBank Accession No. AAA24940 | | |
| SEQ ID NO: 21 | Signal peptide relating to outer membrane protein p1 precursor - | 21 | residues |
| | Haemophilus influenzae - GenBank Accession No. AAA24990 | | |
| SEQ ID NO: 22 | Signal peptide relating to outer membrane protein precursor - | 20 | residues |
| | Haemophilus influenzae - GenBank Accession No. AAA24993 | | |
| SEQ ID NO: 23 | Signal peptide relating to major outer membrane protein precursor - | 22 | residues |
| | Neisseria gonorrhoeae - GenBank Accession No. AAA25458 | | |
| SEQ ID NO: 24 | Signal peptide relating to lipoprotein I precursor - Pseudomonas | 24 | residues |
| | aeruginosa - GenBank Accession No. AAA25880 | | |
| SEQ ID NO: 25 | Signal peptide relating to porin protein F precursor - | 24 | residues |
| | Pseudomonas aeruginosa - GenBank Accession No. AAA25973 | | |
| SEQ ID NO: 26 | Signal peptide relating to outer membrane protein - Serratia | 25 | residues |
| | marcescens - GenBank Accession No. AAA26566 | | |
| SEQ ID NO: 27 | Signal peptide relating to serine protease precursor - Serratia | 27 | residues |
| | marcescens - GenBank Accession No. AAA26572 | | |
| SEQ ID NO: 28 | Signal peptide relating to outer membrane protein precursor II - | 21 | residues |
| | Salmonella typhimurium - GenBank Accession No. AAA27169 | | |
| SEQ ID NO: 29 | Signal peptide relating to cationic outer membrane protein | 20 | residues |
| | precursor (gtg start codon) - Salmonella typhimurium - GenBank | | |
| | Accession No. AAA27170 | | |
| SEQ ID NO: 30 | Signal peptide relating to ferrienterochelin receptor protein - | 22 | residues |
| | Escherichia coli - GenBank Accession No. AAA65994 | | |
| SEQ ID NO: 31 | Signal peptide relating to outer membrane protein A - Cloning | 21 | residues |
| | vector pINIIIompA3 - GenBank Accession No. AAA82946 | | |
| SEQ ID NO: 32 | Signal peptide relating to lambda receptor protein - Escherichia | 25 | residues |
| | coli - GenBank Accession No. AAB59058 | | |
| SEQ ID NO: 33 | Signal peptide relating to periplasmic maltose-binding protein - | 26 | residues |
| | Escherichia coli - GenBank Accession No. AAB59056 | | |
| SEQ ID NO: 34 | Signal peptide relating to Opal 1 - Neisseria meningitidis - | 32 | residues |
| | GenBank Accession No. AAC44565 | | |
| SEQ ID NO: 35 | Signal peptide relating to Opal 2 - Neisseria meningitidis - | 26 | residues |
| | GenBank Accession No. AAC44566 | | |
| SEQ ID NO: 36 | Signal peptide relating to H.8 outer membrane protein precursor - | 21 | residues |
| | Neisseria gonorrhoeae - GenBank Accession No. P07211 | | |
| SEQ ID NO: 37 | Signal peptide relating to Immunoglobulin A1 protease precursor | 25 | residues |
| | (IgA1 protease). - Haemophilus influenzae - GenBank Accession | | |
| | No. P42782 | | |
| SEQ ID NO: 38 | Signal peptide relating to outer membrane porin OmpC precursor - | 21 | residues |
| | Escherichia coli - GenBank Accession No. MMECPC | | |
| SEQ ID NO: 39 | Signal peptide relating to HrpA - Ralstonia solanacearum - | 16 | residues |
| | GenBank Accession No. CAB58261 | | |
| SEQ ID NO: 40 | Signal peptide relating to putative secreted protein - Streptomyces | 23 | residues |
| | coelicolor A3(2) - GenBank Accession No. CAB92608 | | |
| SEQ ID NO: 41 | Signal peptide relating to outer membrane porin OmpF precursor - | 22 | residues |
| | Escherichia coli - GenBank Accession No. MMECF | | |
| SEQ ID NO: 42 | Signal peptide relating to ORF2a precursor - Brucella melitensis | 22 | residues |
| | biovar Abortus - GenBank Accession No. AAA83993 | | |
| SEQ ID NO: 43 | Signal peptide relating to IgA-specific serine endopeptidase | 27 | residues |
| | precursor - Neisseria gonorrhoeae - GenBank Accession No. | | |
| | AZNHG | | |
| SEQ ID NO: 44 | Signal peptide relating to Maltoporin precursor (Maltose-inducible | 25 | residues |
| | porin) - Escherichia coli - GenBank Accession No. P02943 | | |
| SEQ ID NO: 45 | Signal peptide relating to adhesion and penetration protein | 25 | residues |
| | precursor - Haemophilus influenzae - GenBank Accession No. | | |
| | P45387 | | |
| SEQ ID NO: 46 | Signal peptide relating to adhesion and penetration protein | 25 | residues |
| | precursor 2 - Haemophilus influenzae - GenBank Accession No. | | |
| | P44596 | | |
| SEQ ID NO: 47 | Signal peptide relating to outer membrane protein F precursor | 22 | residues |
| | (Porin OmpF) - Escherichia coli - GenBank Accession No. | | |
| | P02931 | | |
| SEQ ID NO: 48 | Signal peptide relating to outer membrane protein C precursor | 21 | residues |
| | (Porin OmpC) - Escherichia coli - GenBank Accession No. | | |
| | P06996 | | |
| SEQ ID NO: 49 | Signal peptide relating to Porin-like protein BU359 precursor - | 26 | residues |
| | Buchnera aphidicola (Acyrthosiphon pisum) - GenBank | | |
| | Accession No. P57440 | | |
| SEQ ID NO: 50 | Signal peptide relating to outer membrane protein C precursor - | 21 | residues |
| | (Porin OmpC) - Salmonella typhimurium - GenBank Accession | | |
| | No. O52503 | | |
| SEQ ID NO: 51 | Signal peptide relating to outer membrane pore protein E | 23 | residues |
| | precursor - Escherichia coli - Genbank Accession No. P02932 | | |
| SEQ ID NO: 52 | Signal peptide relating to outer membrane porin protein LC | 23 | residues |
| | precursor - Bacteriophage PA-2 - GenBank Accession No. | | |
| | P07238 | | |
| SEQ ID NO: 53 | Signal peptide relating to outer membrane porin protein OmpD | 21 | residues |
| | precursor - Salmonella typhimurium - GenBank Accession No. | | |
| | P37592 | | |
| SEQ ID NO: 54 | Signal peptide relating to outer membrane protein 2 - Salmonella | 22 | residues |
| | enterica subsp. enterica serovar Typhi - GenBank Accession No. | | |
| | NP_456059 | | |
| SEQ ID NO: 55 | Signal peptide relating to outer membrane protein S1 - Salmonella | 22 | residues |
| | enterica subsp. enterica serovar Typhi - GenBank Accession No. | | |
| | NP_456554 | | |
| SEQ ID NO: 56 | Signal peptide relating to outer membrane protein C - Salmonella | 22 | residues |
| | enterica subsp. enterica serovar Typhi - GenBank Accession No. | | |
| | NP_456812 | | |
| SEQ ID NO: 57 | Signal peptide relating to outer membrane protein F precursor - | 22 | residues |
| | Salmonella typhi - GenBank Accession No. Q56113 | | |
| SEQ ID NO: 58 | Signal peptide relating to outer membrane pore protein E | 23 | residues |
| | precursor 2 - Salmonella typhi - GenBank Accession No. Q56119 | | |
| SEQ ID NO: 59 | Signal peptide relating to outer membrane protein 1b (Ib; c) - | 22 | residues |
| | Escherichia coli O157: H7 EDL933 - GenBank Accession No. | | |
| | NP_288795 | | |
| SEQ ID NO: 60 | Signal peptide relating to outer membrane protein C2 - Yersinia | 22 | residues |
| | pestis - GenBank Accession No. NP_404809 | | |
| SEQ ID NO: 61 | Signal peptide relating to outer membrane protein C, porin - | 25 | residues |
| | Yersinia pestis - GenBank Accession No. NP_404824 | | |
| SEQ ID NO: 62 | Signal peptide relating to putative outer membrane porin C protein - | 23 | residues |
| | Yersinia pestis - GenBank Accession No. NP_405004 | | |
| SEQ ID NO: 63 | Signal peptide relating to outer membrane protein F precursor - | 23 | residues |
| | Salmonella enterica subsp. enterica serovar Typhi | | |
| | GenBank Accession No. NP_455485 | | |
| SEQ ID NO: 64 | Signal peptide relating to outer membrane protein S2 precursor - | 21 | residues |
| | Salmonella typhi - GenBank Accession No. Q56111 | | |
| SEQ ID NO: 65 | Signal peptide relating to outer membrane protein S1 precursor - | 21 | residues |
| | Salmonella typhi - GenBank Accession No. Q56110 | | |
| SEQ ID NO: 66 | Signal peptide relating to Outer membrane protein C precursor - | 23 | residues |
| | Salmonella typhi - GenBank Accession No. P09878 | | |
| SEQ ID NO: 67 | Signal peptide relating to outer membrane protein A precursor - | 22 | residues |
| | Klebsiella pneumoniae - GenBank Accession No. JC6558 | | |
| SEQ ID NO: 68 | Signal peptide relating to outer membrane protein (ompA) - | 22 | residues |
| | Salmonella typhimurium - GenBank Accession No. CAA26037 | | |
| SEQ ID NO: 69 | Signal peptide relating to OmpA protein - Enterobacter | 22 | residues |
| | aerogenes - GenBank Accession No. CAA25062 | | |
| SEQ ID NO: 70 | Signal peptide relating to outer membrane protein 3a (11*; G; d) - | 22 | residues |
| | Escherichia coli - GenBank Accession No. NP_286832 | | |
| SEQ ID NO: 71 | Signal peptide relating to outer membrane protein A precursor 2 - | 22 | residues |
| | Shigella dysenteriae - GenBank Accession No. MMEBAD | | |
| SEQ ID NO: 72 | Signal peptide relating to outer membrane protein ompA precursor - | 22 | residues |
| | Serratia marcescens - GenBank Accession No. S07298 | | |
| SEQ ID NO: 73 | Signal peptide relating to putative outer-membrane protein A - | 22 | residues |
| | Erwinia carotovora - GenBank Accession No. CAB57308 | | |
| SEQ ID NO: 74 | Signal peptide relating to putative outer membrane porin A | 22 | residues |
| | protein - Yersinia pestis - GenBank Accession No. NP_405026 | | |
| SEQ ID NO: 75 | Signal peptide relating to OmpA - Pasteurella multocida - | 22 | residues |
| | GenBank Accession No. AAK61593 | | |
| SEQ ID NO: 76 | Signal peptide relating to outer membrane protein A precursor 3 - | 22 | residues |
| | Buchnera sp. APS - GenBank Accession No. - | | |
| | GenBank Accession No. NP_240151 | | |
| SEQ ID NO: 77 | Signal peptide relating to OmpA2 - Haemophilus ducreyi - | 25 | residues |
| | GenBank Accession No. AAB4927 | | |
| SEQ ID NO: 78 | Signal peptide relating to Outer membrane protein - Haemophilus | 22 | residues |
| | sp. - GenBank Accession No. CAA07454 | | |
| SEQ ID NO: 79 | Signal peptide relating to outer membrane protein A 2 - Bacillus | 27 | residues |
| | subtilis - GenBank Accession No. I39969 | | |
| SEQ ID NO: 80 | Signal peptide relating to major outer membrane protein - | 25 | residues |
| | Haemophilus ducreyi - GenBank Accession No. AAB49273 | | |
| SEQ ID NO: 81 | Signal peptide relating to hypothetical protein - Vibrio sp. - | 22 | residues |
| | GenBank Accession No. CAC40971 | | |
| SEQ ID NO: 82 | Signal peptide relating to outer membrane protein P5 (ompA)- | 22 | residues |
| | Haemophilus influenzae Rd - GenBank Accession No. | | |
| | NP_439322 | | |
| SEQ ID NO: 83 | Signal peptide relating to fimbrial protein - Haemophilus | 22 | residues |
| | influenzae - GenBank Accession No. AAA24959 | | |
| SEQ ID NO: 84 | Signal peptide relating to signal peptide sequence - Pasteurella | 22 | residues |
| | multocida - GenBank Accession No. NP_245723 | | |
| SEQ ID NO: 85 | Signal peptide relating to outer membrane protein PomA - | 25 | residues |
| | Mannheimia haemolytica - GenBank Accession No. AAD53408 | | |
| SEQ ID NO: 86 | Signal peptide relating to hypothetical signal peptide protein - | 24 | residues |
| | Sinorhizobium meliloti - GenBank Accession No. NP_385333 | | |
| SEQ ID NO: 87 | Signal peptide relating to outer membrane protein 34; Omp34 - | 22 | residues |
| | Actinobacillus actinomycetemcomitans - GenBank Accession No. | | |
| | AAC00068 | | |
| SEQ ID NO: 88 | Signal peptide relating to US 5,284,768 - artificial sequence | 22 | residues |
| SEQ ID NO: 89 | Signal peptide relating to US 5,712,114-1 - artificial sequence | 22 | residues |
| SEQ ID NO: 90 | Signal peptide relating to US 5,712,114-2 - artificial sequence | 22 | residues |
| SEQ ID NO: 91 | Nucleotide sequence encoding the signal peptide sequence set | 102 | bases |
| | forth in SEQ ID NO: 3 | | |
| SEQ ID NO: 92 | Nucleotide sequence encoding the signal peptide sequence set | 66 | bases |
| | forth in SEQ ID NO: 4 | | |
| SEQ ID NO: 93 | Nucleotide sequence encoding the signal peptide sequence set | 66 | bases |
| | forth in SEQ ID NO: 5 | | |
| SEQ ID NO: 94 | Nucleotide sequence encoding the signal peptide sequence set | 60 | bases |
| | forth in SEQ ID NO: 6 | | |
| SEQ ID NO: 95 | Nucleotide sequence encoding the signal peptide sequence set | 63 | bases |
| | forth in SEQ ID NO: 7 | | |
| SEQ ID NO: 96 | Nucleotide sequence encoding the signal peptide sequence set | 66 | bases |
| | forth in SEQ ID NO: 8 | | |
| SEQ ID NO: 97 | Nucleotide sequence encoding the signal peptide sequence set | 81 | bases |
| | forth in SEQ ID NO: 9 | | |
| SEQ ID NO: 98 | Nucleotide sequence encoding the signal peptide sequence set | 72 | bases |
| | forth in SEQ ID NO: 10 | | |
| SEQ ID NO: 99 | Nucleotide sequence encoding the signal peptide sequence set | 69 | bases |
| | forth in SEQ ID NO: 11 | | |
| SEQ ID NO: 100 | Nucleotide sequence encoding the signal peptide sequence set | 66 | bases |
| | forth in SEQ ID NO: 12 | | |
| SEQ ID NO: 101 | Nucleotide sequence encoding the signal peptide sequence set | 60 | bases |
| | forth in SEQ ID NO: 13 | | |
| SEQ ID NO: 102 | Nucleotide sequence encoding the signal peptide sequence set | 66 | bases |
| | forth in SEQ ID NO: 14 | | |
| SEQ ID NO: 103 | Nucleotide sequence encoding the signal peptide sequence set | 72 | bases |
| | forth in SEQ ID NO: 15 | | |
| SEQ ID NO: 104 | Nucleotide sequence encoding the signal peptide sequence set | 72 | bases |
| | forth in SEQ ID NO: 16 | | |
| SEQ ID NO: 105 | Nucleotide sequence encoding the signal peptide sequence set | 75 | bases |
| | forth in SEQ ID NO: 17 | | |
| SEQ ID NO: 106 | Nucleotide sequence encoding the signal peptide sequence set | 81 | bases |
| | forth in SEQ ID NO: 18 | | |
| SEQ ID NO: 107 | Nucleotide sequence encoding the signal peptide sequence set | 63 | bases |
| | forth in SEQ ID NO: 19 | | |
| SEQ ID NO: 108 | Nucleotide sequence encoding the signal peptide sequence set | 60 | bases |
| | forth in SEQ ID NO: 20 | | |
| SEQ ID NO: 109 | Nucleotide sequence encoding the signal peptide sequence set | 66 | bases |
| | forth in SEQ ID NO: 21 | | |
| SEQ ID NO: 110 | Nucleotide sequence encoding the signal peptide sequence set | 63 | bases |
| | forth in SEQ ID NO: 22 | | |
| SEQ ID NO: 111 | Nucleotide sequence encoding the signal peptide sequence set | 75 | bases |
| | forth in SEQ ID NO: 23 | | |
| SEQ ID NO: 112 | Nucleotide sequence encoding the signal peptide sequence set | 78 | bases |
| | forth in SEQ ID NO: 24 | | |
| SEQ ID NO: 113 | Nucleotide sequence encoding the signal peptide sequence set | 98 | bases |
| | forth in SEQ ID NO: 25 | | |
| SEQ ID NO: 114 | Nucleotide sequence encoding the signal peptide sequence set | 78 | bases |
| | forth in SEQ ID NO: 26 | | |
| SEQ ID NO: 115 | Nucleotide sequence encoding the signal peptide sequence set | 48 | bases |
| | forth in SEQ ID NO: 30 | | |
| SEQ ID NO: 116 | Nucleotide sequence encoding the signal peptide sequence set | 69 | bases |
| | forth in SEQ ID NO: 31 | | |
| SEQ ID NO: 117 | Nucleotide sequence encoding the signal peptide sequence set | 66 | bases |
| | forth in SEQ ID NO: 33 | | |
| SEQ ID NO: 118 | Nucleotide sequence encoding the signal peptide sequence set | 66 | bases |
| | forth in SEQ ID NO: 45 | | |
| SEQ ID NO: 119 | Nucleotide sequence encoding the signal peptide sequence set | 66 | bases |
| | forth in SEQ ID NO: 46 | | |
| SEQ ID NO: 120 | Nucleotide sequence encoding the signal peptide sequence set | 66 | bases |
| | forth in SEQ ID NO: 47 | | |
| SEQ ID NO: 121 | Nucleotide sequence encoding the signal peptide sequence set | 66 | bases |
| | forth in SEQ ID NO: 50 | | |
| SEQ ID NO: 122 | Nucleotide sequence encoding the signal peptide sequence set | 66 | bases |
| | forth in SEQ ID NO: 59 | | |
| SEQ ID NO: 123 | Nucleotide sequence encoding the signal peptide sequence set | 66 | bases |
| | forth in SEQ ID NO: 60 | | |
| SEQ ID NO: 124 | Nucleotide sequence encoding the signal peptide sequence set | 66 | bases |
| | forth in SEQ ID NO: 64 | | |
| SEQ ID NO: 125 | Nucleotide sequence encoding the signal peptide sequence set | 66 | bases |
| | forth in SEQ ID NO: 66 | | |
| SEQ ID NO: 126 | Nucleotide sequence encoding the signal peptide sequence set | 75 | bases |
| | forth in SEQ ID NO: 68 | | |
| SEQ ID NO: 127 | Nucleotide sequence encoding the signal peptide sequence set | 66 | bases |
| | forth in SEQ ID NO: 69 | | |
| SEQ ID NO: 128 | Nucleotide sequence encoding the signal peptide sequence set | 75 | bases |
| | forth in SEQ ID NO: 71 | | |
| SEQ ID NO: 129 | Nucleotide sequence encoding the signal peptide sequence set | 84 | bases |
| | forth in SEQ ID NO: 72 | | |
| SEQ ID NO: 130 | Nucleotide sequence encoding the signal peptide sequence set | 66 | bases |
| | forth in SEQ ID NO: 74 | | |
| SEQ ID NO: 131 | Nucleotide sequence encoding the signal peptide sequence set | 75 | bases |
| | forth in SEQ ID NO: 76 | | |
| SEQ ID NO: 132 | Nucleotide sequence encoding the signal peptide sequence set | 66 | bases |
| | forth in SEQ ID NO: 78 | | |
| SEQ ID NO: 133 | Portable N-terminal SCE | 36 | residues |
| SEQ ID NO: 134 | Portable C-terminal SCE | 36 | residues |
| SEQ ID NO: 135 | Nucleic acid sequence encoding N-SCE | 63 | bases |
| SEQ ID NO: 136 | N-SCE | 21 | residues |
| SEQ ID NO: 137 | Nucleic acid sequence encoding SCE-C | 63 | bases |
| SEQ ID NO: 138 | SCE-C | 21 | residues |
| SEQ ID NO: 139 | Nucleic acid sequence encoding murine GM-SCF | 420 | bases |
| SEQ ID NO: 140 | Amino acid sequence for murine GM-SCF | 140 | residues |
| SEQ ID NO: 141 | Nucleic acid sequence encoding human GM-CSF | 429 | bases |
| SEQ ID NO: 142 | Amino acid sequence for human GM-CSF | 143 | residues |
| SEQ ID NO: 143 | Nucleic acid sequence encoding murine IFN-β | 543 | bases |
| SEQ ID NO: 144 | Amino acid sequence for murine IFN-β | 181 | residues |
| SEQ ID NO: 145 | Nucleic acid sequence encoding human IFN-β | 558 | bases |
| SEQ ID NO: 146 | Amino acid sequence for human IFN-β | 186 | residues |
| SEQ ID NO: 147 | Nucleic acid sequence encoding murine IL-1Ra | 531 | bases |
| SEQ ID NO: 148 | Amino acid sequence for murine IL-1Ra | 177 | residues |
| SEQ ID NO: 149 | Nucleic acid sequence encoding human IL-1Ra | 528 | bases |
| SEQ ID NO: 150 | Amino acid sequence for human IL-1Ra | 176 | residues |
| SEQ ID NO: 151 | Nucleic acid sequence encoding murine IL-2 | 504 | bases |
| SEQ ID NO: 152 | Amino acid sequence for murine IL-2 | 152 | residues |
| SEQ ID NO: 153 | Nucleic acid sequence encoding human IL-2 | 396 | bases |
| SEQ ID NO: 154 | Amino acid sequence for human IL-2 | 132 | residues |
| SEQ ID NO: 155 | Nucleic acid sequence encoding murine Fas ligand | 834 | bases |
| SEQ ID NO: 156 | Amino acid sequence for murine Fas ligand | 278 | residues |
| SEQ ID NO: 157 | Nucleic acid sequence encoding human Fas ligand | 840 | bases |
| SEQ ID NO: 158 | Amino acid sequence for human Fas ligand | 280 | residues |
| SEQ ID NO: 159 | Nucleic acid sequence encoding HEL | 438 | bases |
| SEQ ID NO: 160 | Amino acid sequence for HEL | 146 | residues |
| SEQ ID NO: 161 | Nucleic acid sequence encoding Flag tag | 24 | bases |
| SEQ ID NO: 162 | Amino acid sequence for Flag tag | 8 | residues |
| SEQ ID NO: 163 | Nucleic acid sequence encoding His tag | 18 | bases |
| SEQ ID NO: 164 | Amino acid sequence for His tag | 6 | residues |
| SEQ ID NO: 165 | Nucleic acid sequence encoding Strep tag | 27 | bases |
| SEQ ID NO: 166 | Amino acid sequence for Strep tag | 9 | residues |
| SEQ ID NO: 167 | Amino acid sequence for Spacer 1 | 5-55 | residues |
| SEQ ID NO: 168 | Amino acid sequence for Spacer 2 | 3 | residues |
| SEQ ID NO: 169 | Amino acid sequence for Spacer 3 | 5-55 | residues |
| SEQ ID NO: 170 | Nucleic acid sequence encoding Spacer 1, n = 0 | 15 | bases |
| SEQ ID NO: 171 | Amino acid sequence for Spacer 1, n = 0 | 5 | residues |
| SEQ ID NO: 172 | Nucleic acid sequence encoding Spacer 1, n = 1 | 30 | bases |
| SEQ ID NO: 173 | Amino acid sequence for Spacer 1, n = 1 | 10 | residues |
| SEQ ID NO: 174 | Nucleic acid sequence encoding Spacer 1, n = 2 | 45 | bases |
| SEQ ID NO: 175 | Amino acid sequence for Spacer 1, n = 2 | 15 | residues |
| SEQ ID NO: 176 | Nucleic acid sequence encoding Spacer 1, n 3 | 60+ | bases |
| SEQ ID NO: 177 | Nucleic acid sequence encoding Spacer 2 | 9 | bases |
| SEQ ID NO: 178 | Nucleic acid sequence encoding Spacer 3, n = 0 | 15 | bases |
| SEQ ID NO: 179 | Amino acid sequence for Spacer 3, n = 0 | 5 | residues |
| SEQ ID NO: 180 | Nucleic acid sequence encoding Spacer 3, n = 1 | 30 | bases |
| SEQ ID NO: 181 | Amino acid sequence for Spacer 3, n = 1 | 10 | residues |
| SEQ ID NO: 182 | Nucleic acid sequence encoding Spacer 3, n = 2 | 45 | bases |
| SEQ ID NO: 183 | Amino acid sequence for Spacer 3, n = 2 | 15 | residues |
| SEQ ID NO: 184 | Nucleic acid sequence encoding Spacer 3, n 3 | 60+ | bases |
| SEQ ID NO: 185 | Nucleic acid sequence encoding self-coalescing murine GM-CSF | 552 | bases |
| | chimeric construct | | |
| SEQ ID NO: 186 | Amino acid sequence encoded by SEQ ID NO: 185 | 182 | residues |
| SEQ ID NO: 187 | Nucleic acid sequence encoding self-coalescing human GM-CSF | 579 | bases |
| | chimeric construct | | |
| SEQ ID NO: 188 | Amino acid sequence encoded by SEQ ID NO: 187 | 191 | residues |
| SEQ ID NO: 189 | Nucleic acid sequence encoding self-coalescing murine IFN-beta | 732 | bases |
| | chimeric construct | | |
| SEQ ID NO: 190 | Amino acid sequence encoded by SEQ ID NO: 190 | 242 | residues |
| SEQ ID NO: 191 | Nucleic acid sequence encoding self-coalescing human IFN-beta | 708 | bases |
| | chimeric construct | | |
| SEQ ID NO: 192 | Amino acid sequence encoded by SEQ ID NO: 191 | 234 | residues |
| SEQ ID NO: 193 | Nucleic acid sequence encoding self-coalescing murine IL- 1Ra | 723 | bases |
| | chimeric construct | | |
| SEQ ID NO: 194 | Amino acid sequence encoded by SEQ ID NO: 193 | 239 | residues |
| SEQ ID NO: 195 | Nucleic acid sequence encoding self-coalescing human IL-1Ra | 642 | bases |
| | chimeric construct | | |
| SEQ ID NO: 196 | Amino acid sequence encoded by SEQ ID NO: 195 | 212 | residues |
| SEQ ID NO: 197 | Nucleic acid sequence encoding self-coalescing murine IL-2 | 642 | bases |
| | chimeric construct | | |
| SEQ ID NO: 198 | Amino acid sequence encoded by SEQ ID NO: 197 | 212 | residues |
| SEQ ID NO: 199 | Nucleic acid sequence encoding self-coalescing human IL-2 | 513 | bases |
| | chimeric construct | | |
| SEQ ID NO: 200 | Amino acid sequence encoded by SEQ ID NO: 199 | 169 | residues |
| SEQ ID NO: 201 | Nucleic acid sequence encoding self-coalescing murine Fas-L | 960 | bases |
| | chimeric construct | | |
| SEQ ID NO: 202 | Amino acid sequence encoded by SEQ ID NO: 201 | 318 | residues |
| SEQ ID NO: 203 | Nucleic acid sequence encoding self-coalescing human Fas-L | 993 | bases |
| | chimeric construct | | |
| SEQ ID NO: 204 | Amino acid sequence encoded by SEQ ID NO: 203 | 329 | residues |
| SEQ ID NO: 205 | Nucleic acid sequence encoding self-coalescing HEL chimeric | 633 | bases |
| | construct | | |
| SEQ ID NO: 206 | Amino acid sequence encoded by SEQ ID NO: 205 | 209 | residues |
| SEQ ID NO: 207 | Nucleic acid sequence encoding self-coalescing mouse MCP-1 | 282 | bases |
| SEQ ID NO: 208 | Amino acid sequence encoded by SEQ ID NO: 207 | 94 | residues |
| SEQ ID NO: 209 | Nucleic acid sequence encoding self-coalescing human MCP-1 | 294 | bases |
| SEQ ID NO: 210 | Amino acid sequence encoded by SEQ ID NO: 209 | 98 | residues |
| SEQ ID NO: 211 | Nucleic acid sequence encoding self-coalescing murine MCP-1 | 402 | bases |
| | chimeric construct | | |
| SEQ ID NO: 212 | Amino acid sequence encoded by SEQ ID NO: 211 | 132 | residues |
| SEQ ID NO: 213 | Nucleic acid sequence encoding self-coalescing human MCP-1 | 405 | bases |
| | chimeric construct | | |
| SEQ ID NO: 214 | Amino acid sequence encoded by SEQ ID NO: 213 | 133 | residues |
| SEQ ID NO: 215 | Amino acid sequence of NCE-2 | 20 | residues |
| SEQ ID NO: 216 | Amino acid sequence of a human ACTH chimeric peptide | 69 | residues |
| SEQ ID NO: 217 | Amino acid sequence of a murine ACTH chimeric peptide | 65 | residues |
| SEQ ID NO: 218 | Amino acid sequence of a human alpha MSH chimeric peptide | 44 | residues |
| SEQ ID NO: 219 | Amino acid sequence of a human beta MSH chimeric peptide | 47 | residues |
| SEQ ID NO: 220 | Amino acid sequence of a murine beta MSH chimeric peptide | 52 | residues |
| SEQ ID NO: 221 | Amino acid sequence of a human gamma MSH chimeric peptide | 37 | residues |
| SEQ ID NO: 222 | Amino acid sequence of a human angiotensin I chimeric peptide | 40 | residues |
| SEQ ID NO: 223 | Amino acid sequence of a human angiotensin II chimeric peptide | 39 | residues |
| SEQ ID NO: 224 | Amino acid sequence of a human angiotensin III chimeric peptide | 37 | residues |
| SEQ ID NO: 225 | Amino acid sequence of a human GHRH chimeric peptide I | 55 | residues |
| SEQ ID NO: 226 | Amino acid sequence of a human GHRH chimeric peptide I | 70 | residues |
| SEQ ID NO: 227 | Amino acid sequence of a murine GHRH chimeric peptide | 67 | residues |
| SEQ ID NO: 228 | Amino acid sequence of a human IL-1 beta chimeric peptide I | 35 | residues |
| SEQ ID NO: 229 | Amino acid sequence of a human IL-1 beta chimeric peptide II | 60 | residues |
| SEQ ID NO: 230 | Amino acid sequence of a human IL-2 chimeric peptide I | 38 | residues |
| SEQ ID NO: 231 | Amino acid sequence of a human IL-2 chimeric peptide II | 44 | residues |
| SEQ ID NO: 232 | Amino acid sequence of a human IL-2 chimeric peptide III | 41 | residues |
| SEQ ID NO: 233 | Amino acid sequence of a human TNF-alpha chimeric peptide I | 43 | residues |
| SEQ ID NO: 234 | Amino acid sequence of a human TNF-alpha chimeric peptide II | 52 | residues |
| SEQ ID NO: 235 | Amino acid sequence of a human TNF-alpha chimeric peptide III | 46 | residues |
| SEQ ID NO: 236 | Amino acid sequence of a human Cys-BAFF-R chimeric peptide I | 54 | residues |
| SEQ ID NO: 237 | Amino acid sequence of a human Cys-BAFF-R chimeric peptide II | 56 | residues |
| SEQ ID NO: 238 | Amino acid sequence of a human P55-TNF-R chimeric peptide | 42 | residues |
| SEQ ID NO: 239 | Amino acid sequence of a human P75-TNF-R chimeric peptide | 51 | residues |
| SEQ ID NO: 240 | Amino acid sequence of a human IL-6-R chimeric peptide | 43 | residues |
| SEQ ID NO: 241 | Amino acid sequence of a L-selectin chimeric peptide | 47 | residues |
| SEQ ID NO: 242 | Amino acid sequence of a MUC-1 chimeric peptide | 50 | residues |
| SEQ ID NO: 243 | Amino acid sequence of a ovalbumin chimeric peptide I | 48 | residues |
| SEQ ID NO: 244 | Amino acid sequence of a ovalbumin chimeric peptide II | 38 | residues |
| SEQ ID NO: 245 | Amino acid sequence of a HIV gp120 chimeric peptide I | 51 | residues |
| SEQ ID NO: 246 | Amino acid sequence of a HIV gp120 chimeric peptide II | 49 | residues |
| SEQ ID NO: 247 | Amino acid sequence of a HIV gp120 chimeric peptide III | 54 | residues |
| SEQ ID NO: 248 | Amino acid sequence of a HIV gp41 chimeric peptide | 66 | residues |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, preferably by as much as 20%, and more preferably by as much as 10% to a reference quantity, level, value, dimension, size, or amount.

The term “activity” as used herein describes the activity of a non-aggregated molecule of interest. Thus, for example, a higher order aggregate of a molecule of interest has activity if the aggregate exhibits the activity of the non aggregated molecule.

“Bifunctional crosslinking reagent” means a reagent containing two reactive groups, the reagent thereby having the ability to covalently link two target groups. The reactive groups in a crosslinking reagent typically belong to the classes of functional groups including succinimidyl esters, maleimides and haloacetamides such as iodoacetamides.

By “biologically active fragment” is meant a fragment of a full-length parent polypeptide which fragment retains an activity of that polypeptide. For example, a biologically active fragment of a self-coalescing element will coalesce with compatible self-coalescing elements that are either identical or sufficiently similar to permit co-aggregation with each other into higher order aggregates. As used herein, the term “biologically active fragment” includes deletion mutants and small peptides, for example of at least 6, 7, 8, 9, 10, 11, 12, 13, 14, 15, 16, 17, 18, 19, 20, 21, 22, 23, 24, 25, 26, 27, 28, 29, 30 contiguous amino acids, which comprise an activity of a parent polypeptide. Fragments of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “expression vector” is meant any autonomous genetic element capable of directing the synthesis of a protein encoded by the vector. Such expression vectors are known to practitioners in the art.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules.

By “effective amount”, in the context of modulating an activity or of treating or preventing a condition is meant the administration of that amount of active to an individual in need of such modulation, treatment or prophylaxis, either in a single dose or as part of a series, that is effective for modulation of that effect or for treatment or prophylaxis of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

“Homobifunctional crosslinking reagent” means a reagent containing identical reactive groups, which is predominantly used to link like target groups such as two thiols or two amines.

“Heterobifunctional crosslinking reagent” means a reagent containing reactive groups having dissimilar chemistry, thereby allowing the formation of crosslinks between unlike functional groups.

By “higher order” is meant an aggregate of at least 10, 12, 15, 20, 25, 50, 75, 100, 150, 200, 300, 400, 500, 600, 700, 800, 900, 1000 molecules.

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

By “isolated” is meant material that is substantially or essentially free from components that normally accompany it in its native state. For example, an “isolated polynucleotide”, as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and especially from association with other components of the cell, i.e., it is not associated with in vivo substances.

By “marker gene” is meant a gene that imparts a distinct phenotype to cells expressing the marker gene and thus allows such transformed cells to be distinguished from cells that do not have the marker. A selectable marker gene confers a trait for which one can ‘select’ based on resistance to a selective agent (e.g., a herbicide, antibiotic, radiation, heat, or other treatment damaging to untransformed cells). A screenable marker gene (or reporter gene) confers a trait that one can identify through observation or testing, i.e., by ‘screening’ (e.g., β-glucuronidase, luciferase, or other enzyme activity not present in untransformed cells).

As used herein, a “membrane-translocating sequence” is an amino acid sequence capable of mediating the transport of a polypeptide to an intracellular compartment or location or to the extracellular environment.

By “obtained from” is meant that a sample such as, for example, a nucleic acid extract or polypeptide extract is isolated from, or derived from, a particular source. For example, the extract may be isolated directly from any membrane-translocating sequence-containing organism, such as but not limited to bacteria, yeast and plants as well as animals including mammals, birds, reptiles, fish and insects.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

By “operably linked” is meant that transcriptional and translational regulatory nucleic acids are positioned relative to a polypeptide-encoding polynucleotide in such a manner that the polynucleotide is transcribed and the polypeptide is translated.

The terms “subject” or “individual” or “patient”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, primates, avians, fish, reptiles, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). However, it will be understood that the aforementioned terms do not imply that symptoms are present.

By “pharmaceutically acceptable carrier” is meant a solid or liquid filler, diluent or encapsulating substance that can be safely used in topical or systemic administration to a patient.

The term “polynucleotide” or “nucleic acid” as used herein designates mRNA, RNA, cRNA, cDNA or DNA. The term typically refers to oligonucleotides greater than 30 nucleotides in length.

The terms “polynucleotide variant” and “variant” refer to polynucleotides displaying substantial sequence identity with a reference polynucleotide sequence or polynucleotides that hybridise with a reference sequence under stringent conditions that are defined hereinafter. These terms also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains a biological function or activity of the reference polynucleotide. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides that are distinguished from a reference polypeptide by the addition, deletion or substitution of at least one amino acid. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but can alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 35 nucleotides to several kilobases or more. Primers can be selected to be “substantially complementary” to the sequence on the template to which it is designed to hybridise and serve as a site for the initiation of synthesis. By “substantially complementary”, it is meant that the primer is sufficiently complementary to hybridise with a target polynucleotide. Preferably, the primer contains no mismatches with the template to which it is designed to hybridise but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridisation conditions. Probes can be labelled directly or indirectly.

The terms “purified polypeptide” or “purified protein” and the like means that the polypeptide or protein are substantially free of cellular material or other contaminating proteins from the cell or tissue source from which the protein is derived, or substantially free from chemical precursors or other chemicals when chemically synthesised. “Substantially free” means that a preparation of a chimeric polypeptide of the invention is at least 10% pure. In certain embodiments, the preparation of chimeric polypeptide has less than about 30%, 20%, 10% and more preferably 5% (by dry weight), of non-chimeric polypeptide protein (also referred to herein as a “contaminating protein”), or of chemical precursors or non-chimeric polypeptide chemicals. The invention includes isolated or purified preparations of at least 0.01, 0.1, 1.0, and 10 milligrams in dry weight.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant or synthetic polynucleotide. When the chimeric polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 50 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “self-coalesces” is used herein to refer to a self-coalescing element that may be expected to coalesce with identical polypeptides and also with polypeptides having high similarity (e.g., less than 20% and more preferably less than 10% sequence divergence) but less than complete identity in the amino acid sequence of the self-coalescing element.

By “self-coalescing element”, “SCE” and the like is meant any amino acid sequence which, when conjugated to a molecule of interest, can cause the molecule to coalesce with like molecules into higher order aggregates.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Similarity” refers to the percentage number of amino acids that are identical or constitute conservative substitutions as defined in Table B infra. Similarity may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

“Stringency” as used herein, refers to the temperature and ionic strength conditions, and presence or absence of certain organic solvents, during hybridisation and washing procedures. The higher the stringency, the higher will be the degree of complementarity between immobilised target nucleotide sequences and the labelled probe polynucleotide sequences that remain hybridised to the target after washing.

“Stringent conditions” refers to temperature and ionic conditions under which only nucleotide sequences having a high frequency of complementary bases will hybridise. The stringency required is nucleotide sequence dependent and depends upon the various components present during hybridisation and subsequent washes, and the time allowed for these processes. Generally, in order to maximise the hybridisation rate, non-stringent hybridisation conditions are selected; about 20 to 25° C. lower than the thermal melting point (Tₘ). The Tₘ is the temperature at which 50% of specific target sequence hybridises to a perfectly complementary probe in solution at a defined ionic strength and pH. Generally, in order to require at least about 85% nucleotide complementarity of hybridised sequences, highly stringent washing conditions are selected to be about 5 to 15° C. lower than the Tₘ. In order to require at least about 70% nucleotide complementarity of hybridised sequences, moderately stringent washing conditions are selected to be about 15 to 30° C. lower than the Tₘ. Highly permissive (low stringency) washing conditions may be as low as 50° C. below the Tₘ, allowing a high level of mismatching between hybridised sequences. Those skilled in the art will recognise that other physical and chemical parameters in the hybridisation and wash stages can also be altered to affect the outcome of a detectable hybridisation signal from a specific level of homology between target and probe sequences. Other examples of stringency conditions are described in section 3.3.

The term “transformation” means alteration of the genotype of an organism, for example a bacterium, yeast or plant, by the introduction of a foreign or endogenous nucleic acid.

The term “transgene” is used herein to describe genetic material that has been or is about to be artificially inserted into the genome of a cell, particularly a cell of a living animal. The transgene is used to transform a cell, meaning that a permanent or transient genetic change, desirably a permanent genetic change, is induced in a cell following incorporation of exogenous nucleic acid (usually DNA). A permanent genetic change is generally achieved by introduction of the DNA into the genome of the cell. Vectors for stable integration include plasmids, retroviruses and other animal viruses, YACs (yeast artificial chromosome), BACs (bacterial artificial chromosome) and the like. The transgene is suitably derived from animals including, but not limited to, vertebrates, preferably mammals such as rodents, humans, non-human primates, ovines, bovines, ruminants, lagomorphs, porcines, caprines, equines, canines, felines, aves, etc.

As used herein the term “transgenic” refers to a genetically modified animal in which the endogenous genome is supplemented or modified by the random or site-directed integration of a foreign gene or sequence.

The “transgenic animals” of the invention are suitably produced by experimental manipulation of the genome of the germline of the animal. These genetically engineered animals may be produced by several methods including the introduction of a “transgene” comprising nucleic acid (usually DNA) into an embryonal target cell or integration into a chromosome of the somatic and/or germ line cells of an animal by way of human intervention. A transgenic animal is an animal whose genome has been altered by the introduction of a transgene.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

2. Higher Order Aggregates of the Invention

The present invention extends the application of signal peptide biology beyond the context of protein expression systems and provides diverse and practical applications that employ the self-coalescent property of signal peptides. Not wishing to be bound by any one particular theory or mode of operation, it is believed that the predominantly hydrophobic nature of a signal peptide, at least in part, causes the peptide to coalesce with other like peptides into a higher order multimer or aggregate. Thus, it is proposed in accordance with the present invention that the self-aggregating property of signal peptides can be broadly utilised for coalescing a plurality of the same or different molecules into higher order aggregates with novel or enhanced properties. The aggregates of the present invention find utility in a range of applications, including chemical, therapeutic and prophylactic applications, as described hereafter.

In describing aggregates comprising only identical or substantially similar molecules of interest, the prefix “homo” is used. Enhanced activity, when used in reference to higher order homo-aggregates, includes and encompasses a prolonged half-life (e.g., a longer half-life relative to the naturally occurring or parental molecule of interest), or higher potency (e.g., requiring a smaller quantity relative to the naturally occurring or parental molecule to achieve a specified level of activity). Enhanced activity can also encompass a combination of the above-described activities, e.g., a higher order aggregate with higher potency that also exhibits a prolonged half-life. Tests to determine activity that is specific for a molecule of interest are well-known to those of skill in the art.

The self-coalescing property of signal peptides can also be taken advantage to coalesce different molecules of interest into higher order aggregates. In describing aggregates comprising more than one kind of molecule of interest, the prefix “hetero” is used. For example, a hetero-aggregate comprises two or more molecules of interest with one or more of those molecules being different from one or more of the remaining molecules. Such hetero-aggregates may display the sum of the activities of the non-aggregated molecules of interest. Alternatively, hetero-aggregates may display synergistic characteristics, and thus exhibit an activity greater than the activity that would be exhibited by a similar quantity of each molecule of interest found in the aggregate if each molecular component were to be used alone.

Thus, in one aspect of the present invention, there is provided an isolated or purified higher order aggregate comprising a plurality of chimeric molecules, wherein each chimeric molecule comprises at least one self-coalescing element, which is obtainable or derivable from a membrane translocating sequence or variant thereof, and which is fused, linked or otherwise associated with a molecule of interest, and wherein the or each self-coalescing element is capable of causing an individual chimeric molecule to coalesce with other chimeric molecules into higher order aggregates under conditions favorable to aggregation. Suitably, at least one chimeric molecule of the aggregate is other than a chimeric molecule selected from: (1) a B cell activating fusion protein comprising a B cell surface immunoglobulin binding domain and a signal peptide, wherein a catalytic product of the precursor is capable of inducing B cell mitogenesis; or (2) a fusion protein comprising protein L and ompA, each of which are described in U.S. Pat. No. 6,521,741.

The chimeric molecules of the aggregate may be the same or different and, in this connection, the chimeric molecules may contain the same or different molecules of interest or the same or different self-coalescing elements (SLEs). In a preferred embodiment, the molecule of interest is a polypeptide and, in this context: (1) the term “higher order” is meant to exclude the many proteins that are known to comprise polypeptide dimers, tetramers, or other small numbers of polypeptide subunits in an active complex, (2) the term “higher order aggregate” is meant to exclude random agglomerations of denatured proteins that can form in non-physiological conditions; and (c) the term “self-coalesces” refers to the property of the polypeptide to form ordered aggregates with polypeptides having an identical amino acid sequence under appropriate conditions as taught herein, and is not intended to imply that the coalescing will naturally occur under every concentration or every set of conditions.

2.1 Self-Coalescing Elements

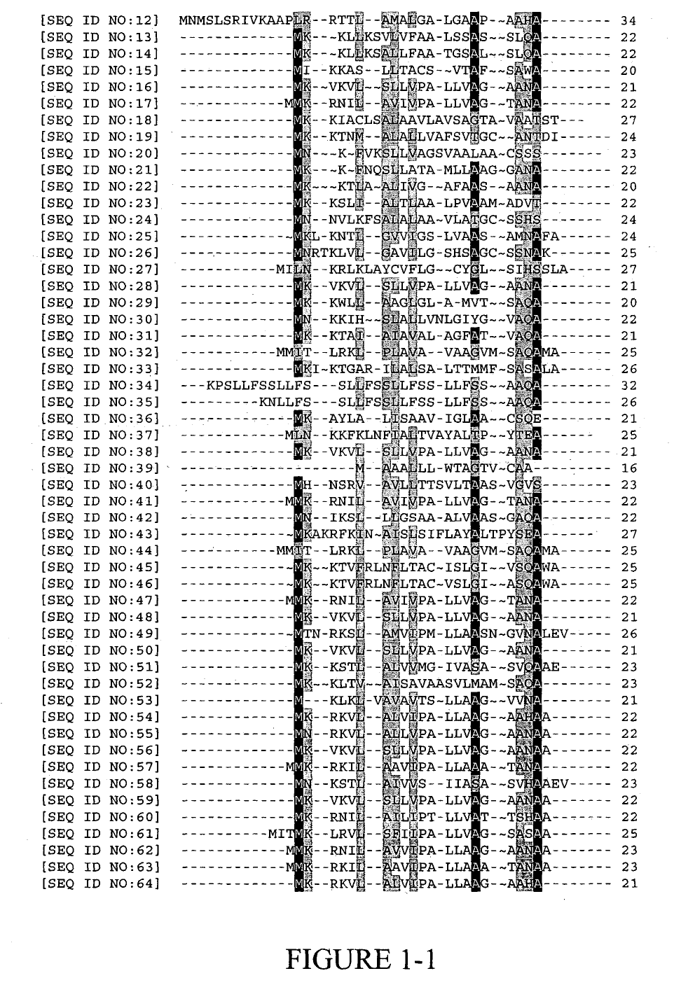

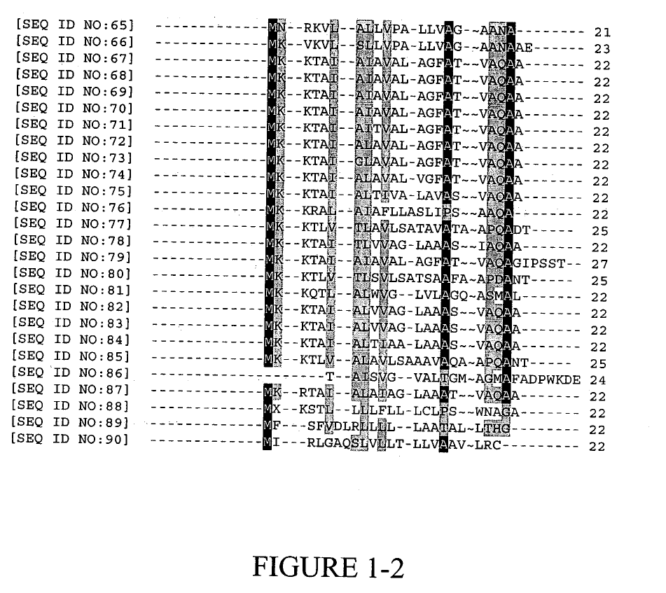

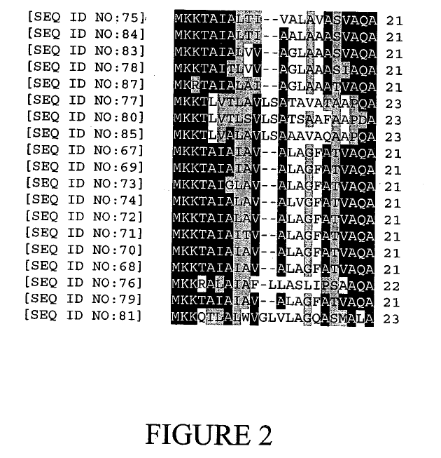

A self-coalescing element (SCE) may consist essentially of about 8 to about 35 amino acid residues, more preferably of about 15 to about 30 amino acid residues of which from about 60 to about 95%, and more suitably from about 70 to about 90%, are small or hydrophobic amino acid residues or modified forms thereof. Usually, one or more polar or charged amino acid residues are located closely adjacent (e.g., within about 5 amino acid residues) to one or both ends of the SCE. A small amino acid residue, which is located at or closely adjacent to (e.g., within about 2 amino acid residues) the carboxyl terminus of the SCE is also desirable. This conservation is illustrated for example in FIG. 1, which shows an alignment of various membrane translocating amino acid sequences from a wide and diverse selection of species. A more pronounced conservation is shown in FIG. 2, which shows an alignment of membrane translocating amino acid sequences of bacterial outer membrane proteins.

In one embodiment, the SCE is represented by the formula:

B₁-X₁[Xⱼ]ₙX₂X₃X₄X₅[Xₖ]ₙX₆[Xₗ]ₙX₇X₈X₉-Z₁ (I)

[SEQ ID NO:1]

wherein: B₁ is absent or is a sequence of n amino acid residues wherein n is from about 1 to about 50 amino acid residues, wherein the sequence comprises the same or different amino acid residues selected from any amino acid residue;

X₁ is a hydrophobic, small, neutral or basic amino acid residue or modified form thereof;

[Xⱼ]ₙ is a sequence of n amino acid residues wherein n is from 0 to 2 amino acid residues and wherein the sequence Xⱼ comprises the same or different amino acid residues selected from any amino acid residue;

X₂ is a hydrophobic, small or polar amino acid residue or modified form thereof;

X₃ is a hydrophobic, small or neutral/polar amino acid residue or modified form thereof;

X₄ is a hydrophobic or small amino acid residue or modified form thereof;

X₅ is a hydrophobic or small amino acid residue or modified form thereof;

[Xₖ]ₖ is a sequence of n amino acid residues wherein n is from 4 to 6 amino acid residues and wherein the sequence Xₖ comprises the same or different amino acid residues selected from a hydrophobic, small, polar or neutral amino acid residue or modified form thereof;

X₆ is a hydrophobic or small amino acid residue or modified form thereof;

[Xₗ]ₙ is a sequence of n amino acid residues wherein n is from 2 to 4 amino acid residues and wherein the sequence X₁ comprises the same or different amino acid residues selected from a hydrophobic, small or polar amino acid residue or modified form thereof;

X₇ is a hydrophobic, small, charged or neutral/polar amino acid residue or modified form thereof;

X₈ is a neutral/polar, charged, hydrophobic, or small amino acid residue or modified form thereof;

X₉ is optional and when present is selected from a small or charged amino acid residue or modified form thereof; and

Z₁ is absent or is a sequence of n amino acid residues wherein n is from about 1 to about 50 amino acid residues, wherein the sequence comprises the same or different amino acid residues selected from any amino acid residue.

Suitably, when B₁ is present, it is a sequence of from about 1 to about 20 amino acid residues. In one embodiment of this type, B₁ is represented by the formula:

B₂J₁[Xᵢ]ₙ (II)

[SEQ ID NO:2]

wherein: B₂ is absent or is a sequence of n amino acid residues wherein n is from about 1 to about 15 amino acid residues, wherein the sequence comprises the same or different amino acid residues selected from any amino acid residue, provided that J₁ is also present;

J₁ is absent or is a hydrophobic, charged, neutral/polar or small amino acid residue or modified form thereof, provided that [Xᵢ]ₙ is also present; and

[Xᵢ]ₙ is a sequence of n amino acid residues wherein n is from 2 to 5 amino acid residues and wherein the sequence Xᵢ comprises the same or different amino acid residues selected from any amino acid residue.

In some embodiments, J₁ is a hydrophobic amino acid residue, e.g., J₁ is selected from Phe and Ile, or modified form thereof. In other embodiments, J₁ is a charged amino acid residue, typically a basic amino acid residue, e.g., J₁ is selected from His, Lys or Arg, or modified form thereof. In still other embodiments, J₁ is a neutral/polar amino acid residue, e.g., Asn, or modified form thereof. In still other embodiments, J₁ is a small amino acid residue, e.g., J₁ is selected from Ser or Thr, or modified form thereof.

In certain embodiments, [Xᵢ]ₙ is represented by the formula:

O₁O₂O₃O₄O₅ (III)

[SEQ ID NO:3]

wherein: at least two of O₁ to O₅ are present, in which:

O₁ is selected from a hydrophobic amino acid residue, e.g., O₁ is selected from Leu or Ile, or modified form thereof, a charged amino acid residue, typically a basic amino acid residue, e.g., Arg, or modified form thereof, a neutral/polar amino acid residue, e.g., Asn, or modified form thereof, or a small amino acid residue, e.g., Ala, or modified form thereof;

O₂ is selected from a small amino acid residue, e.g., Thr, or modified form thereof, or a basic amino acid residue, e.g., Lys, or modified form thereof;

O₃ is selected from a charged (typically basic) amino acid residue, e.g., O₃ is selected from Arg or Lys, or modified form thereof, a neutral/polar amino acid residue, e.g., Asn, or modified form thereof, a hydrophobic amino acid residue, e.g., O₃ is selected from Ile, Val or Leu, or modified form thereof, or a small amino acid residue, e.g., Ala, or modified form thereof,

O₄ is selected from a charged (typically basic) amino acid residue, e.g., O₄ is selected from Arg or Lys, or modified form thereof, a neutral/polar amino acid residue, e.g., O₄ is selected from Gln or Asn, or modified form thereof, a hydrophobic amino acid residue, e.g., O₄ is selected from Phe, Ile, Val or Leu, or modified form thereof, or a small amino acid residue, e.g., O₄ is selected from Ala, Gly, Ser or Thr, or modified form thereof; and

O₅ is selected from a charged (typically basic) amino acid residue, e.g., O₅ is selected from Arg or Lys, or modified form thereof, a neutral/polar amino acid residue, e.g., Asn, or modified form thereof, a hydrophobic amino acid residue, e.g., O₅ is selected from Phe, Ile, Val or Leu, or modified form thereof, or a small amino acid residue, e.g., O₅ is selected from Ala, Gly, Ser or Thr, or modified form thereof.

In some embodiments, X₁ is a hydrophobic amino acid residue e.g., X₁ is selected from Leu, Met, Phe, Ile or Val, or modified form thereof. In other embodiments, X₁ is a small amino acid residue e.g., X₁ is selected from Gly, Ala, Ser or Thr, or modified form thereof. In still other embodiments, X₁ is selected from Cys, Lys or His, or modified form thereof.

In certain embodiment, [Xⱼ]ₙ is a single amino acid residue, which is suitably selected from Ala, Arg, Asn or Val, or modified form thereof. In other embodiments, [Xⱼ]ₙ is a sequence of two amino acid residues, wherein the first amino acid residue is suitably selected from Lys, Asp, Leu, Asn, Ala, Val or Phe, or modified form thereof and wherein the second amino acid residue is suitably selected from Ser, Ala, Lys, Gln, Asn or Leu, or modified form thereof.

In some embodiments, X₂ is a hydrophobic amino acid residue, e.g., X₂ is selected from Val, Leu, Tyr, He or Phe, or modified form thereof. In other embodiments, X₂ is a small amino acid residue, e.g., X₂ is selected from Pro, Ala, Gly, Ser or Thr, or modified form thereof. In still other embodiments, X₂ is selected from Asn or Arg, or modified form thereof.

In some embodiments, X₃ is a small amino acid residue, e.g., X₃ is Ala or modified form thereof. In other embodiments, X₃ is a hydrophobic amino acid residue, e.g., X₃ is selected from Met, Leu, Val, Ile or Phe, or modified form thereof. In still other embodiments, X₃ is Cys or modified form thereof.

In some embodiments, X₄ is a hydrophobic amino acid residue, e.g., X₄ is selected from Val, Leu, Ile or Trp, or modified form thereof. In other embodiments, X₄ is a small amino acid residue, e.g., X₄ is selected from Ala, Gly, Ser or Thr, or modified form thereof.

In some embodiments, X₅ is a small amino acid residue, e.g., X₅ is selected from Ala, Gly, Ser or Thr, or modified form thereof. In other embodiments, X₅ is a hydrophobic amino acid residue, e.g., X₅ is selected from Leu, Phe, Val, Ile, or modified form thereof.

In certain embodiments, [Xₖ]ₙ is represented by the formula:

B₃O₆O₇O₈O₉B₄ (IV)

[SEQ ID NO:4]

wherein: B₃ is selected from a small amino acid residue, e.g., Pro, Ala, Gly, Ser or Thr, or modified form thereof, a hydrophobic amino acid residue, e.g., Val or Leu, or modified form thereof, or a neutral/polar amino acid residue, e.g., Cys, or modified form thereof, at least two of O₆ to O₉ are present, in which:

O₆ is selected from a small amino acid residue, e.g., O₆ is selected from Ala, Gly, Ser or Thr, or modified form thereof, a hydrophobic amino acid residue, e.g., O₆ is selected from Val, Leu, Ile or Met, or modified form thereof, or a neutral/polar amino acid residue, e.g., Cys, or modified form thereof;

O₇ is selected from a small amino acid residue, e.g., O₇ is selected from Ala or Ser, or modified form thereof, a hydrophobic amino acid residue, e.g., Phe, or modified form thereof, or a neutral/polar amino acid residue, e.g., Asn, or modified form thereof;

O₈ is selected from a small amino acid residue, e.g., O₈ is selected from Thr, Ala or Ser, or modified form thereof, or a hydrophobic amino acid residue, e.g., O₈ is selected from Ile, Leu, Val, Met, Phe, Tyr or Trp, or modified form thereof,

O₉ is selected from a small amino acid residue, e.g., O₉ is selected from Pro, Ala, Gly, Ser or Thr, or modified form thereof, a hydrophobic amino acid residue, e.g., O₉ is selected from Ile, Leu, Val or Phe, or modified form thereof, a basic amino acid residue, e.g., His, or modified form thereof, or a neutral/polar amino acid residue, e.g., Cys, or modified form thereof, and

B₄ is selected from a small amino acid residue, e.g., Ala, Ser or Thr, or modified form thereof, or a hydrophobic amino acid residue, e.g., Ile, Val, Leu, Met, Tyr or Phe, or modified form thereof.

In some embodiments, X₆ is a hydrophobic amino acid residue, e.g. X₆ is selected from Leu, Val, Met or Tyr, or modified form thereof. In other embodiments, X₆ is a small amino acid residue, e.g., X₆ is selected from Pro, Ala, Gly, Ser or Thr, or modified form thereof;

In certain embodiments, [Xₗ]ₙ is represented by the formula:

B₅O₁₀O₁₁O₁₂ (V)

[SEQ ID NO:5]

wherein: B₅ is selected from a small amino acid residue, e.g., Pro, Ala, Gly, Ser or Thr, or modified form thereof, a hydrophobic amino acid residue, e.g., Ile, Leu, Val, Phe or Met, or modified form thereof, or a neutral/polar amino acid residue, e.g., Gln, or modified form thereof;

at least one of O₁₀ to O₁₂ are present, in which:

O₁₀ is selected from a small amino acid residue, e.g., O₁₀ is selected from Gly, Ala, Ser or Thr, or modified form thereof, a hydrophobic amino acid residue, e.g., O₁₀ is selected from Val, Leu, Met or Phe, or modified form thereof, a neutral/polar amino acid residue, e.g., O₁₀ is selected from Cys, Asn or Gln, or modified form thereof;

O₁₁ is a small amino acid residue, e.g., Pro, or modified form thereof; and

O₁₂ is selected from a small amino acid residue, e.g., O₁₂ is selected from Ala, Gly, Ser or Thr, or modified form thereof, a hydrophobic amino acid residue, e.g., O₁₂ is selected from Ile, Leu, Val, Tyr or Trp, or modified form thereof, or a neutral/polar amino acid residue, e.g., Cys, or modified form thereof.

In some embodiments, X₇ is a hydrophobic amino acid residue, e.g., X₇ is selected from Leu, Ile, Val or Met, or modified form thereof. In other embodiments, X₇ is a small amino acid residue, e.g., X₇ is selected from Pro, Ala, Gly, Ser or Thr, or modified form thereof. In still other embodiments, X₇ is a charged amino acid residue, e.g., X₇ is selected from Asp or Arg, or modified form thereof. In still other embodiments, X₇ is a neutral/polar amino acid residue, e.g., Asn, or modified form thereof.

In some embodiments, X₈ is a neutral/polar, amino acid residue, e.g., X₈ is selected from Gln, Asn or Cys, or modified form thereof. In other embodiments, X₈ is a charged amino acid residue, e.g., X₈ is selected from His or Glu, or modified form thereof. In still other embodiments, X₈ is a hydrophobic amino acid residue, e.g., X₈ is selected from Val, Met or Trp, or modified form thereof. In still other embodiments, X₈ is a small amino acid residue, e.g., X₈ is selected from Ala or Ser, or modified form thereof.

In some embodiments, X₉ is a small amino acid residue, e.g., X₉ is selected from Ala, Gly, Ser or Thr, or modified form thereof. In other embodiments, X₉ is a charged amino acid residue, more suitably an acidic amino acid residue, e.g., Glu, or modified form thereof.

In certain embodiments, Z₁ is represented by the formula:

J₂J₃J₄Z₂ (VI)

[SEQ ID NO:6]

wherein: J₂ is a small amino acid residue, e.g., Thr, or modified form thereof;

J₃ is absent or is a charged amino acid residue, typically a basic amino acid residue, e.g., Lys, or modified form thereof, provided that J₂ is also present;

J₄ is absent or is a charged amino acid residue, typically a basic amino acid residue, e.g., Lys, or modified form thereof, provided that J₃ is also present; and

Z₂ is absent or is a sequence of n amino acid residues wherein n is from about 1 to about 15 amino acid residues, wherein the sequence comprises the same or different amino acid residues selected from any amino acid residue, provided that J₄ is also present.

Desirably, Z₁ or Z₂ comprise at least 1, 2, 3, 4, 5 charged amino acid residue(s), which are typically, but not exclusively, basic amino acid residues. The charged amino acid residues can be positioned adjacent to each other or can be spaced from one another by one or more other (non-charged) amino acid residues.

In another embodiment, the SCE is represented by the formula:

B₂J₁[Xᵢ]ₙX₁[Xⱼ]ₙX₂X₃X₄X₅[Xₖ]ₙX₆[Xₗ]ₙX₇X₈X₉Z₁ (VII)

[SEQ ID NO:7]

wherein: B₂, J₁, [Xᵢ]ₙ, [Xⱼ]ₙ, [Xₖ]ₙ, [Xₗ]ₙ, X₁₋₉ and Z₁ are as defined above.

In yet another embodiment, the SCE is represented by the formula:

B₁-X₁X₂X₃X₄X₅[Xₘ]ₙX₆X₇X₈X₉X₁₀X₁₁X₁₂X₁₃X₁₄X₁₅X₁₆-Z₁ (VIII)

[SEQ ID NO:8]

wherein: B₁ is absent or is a sequence of n amino acid residues wherein n is from about 1 to about 5 amino acid residues, wherein the sequence comprises the same or different amino acids selected from any amino acid residue;

X₁ is a hydrophobic amino acid residue or modified form thereof;

X₂ is a small amino acid residue or modified form thereof;

X₃ is a hydrophobic amino acid residue or modified form thereof;

X₄ is selected from a hydrophobic or small amino acid residue or modified form thereof;

X₅ is a hydrophobic amino acid residue or modified form thereof; and

[Xₘ]ₙ is a sequence of n amino acid residues wherein n is from 0 to 2 amino acid residues and wherein the sequence Xₘ comprises the same or different amino acid residues selected from a hydrophobic or a small amino acid residue or modified form thereof;

X₆ is a small or hydrophobic amino acid residue or modified form thereof;

X₇ is a hydrophobic or small amino acid residue or modified form thereof;

X₈ is a hydrophobic or small amino acid residue or modified form thereof;

X₉ is a hydrophobic or small amino acid residue or modified form thereof;

X₁₀ is a hydrophobic, small or neutral/polar amino acid residue or modified form thereof;

X₁₁ is a small, hydrophobic or neutral/polar amino acid residue or modified form thereof;

X₁₂ is a small amino acid residue or modified form thereof;

X₁₃ is a hydrophobic or small amino acid residue or modified form thereof;

X₁₄ is a small amino acid residue or modified form thereof;

X₁₅ is a neutral/polar, acidic or hydrophobic amino acid residue or modified form thereof;

X₁₆ is a small amino acid residue or modified form thereof; and

Z₁ is absent or is a sequence of n amino acid residues wherein n is from about 1 to about 20 amino acid residues wherein the sequence comprises the same or different amino acid residues selected from any amino acid residue.

In certain embodiments when B₁ is present, it is represented by the formula:

J₁J₂J₃J₄J₅ (IX)

[SEQ ID NO:9]

wherein: J₁ is absent or is a hydrophobic amino acid residue, e.g., Met, or modified form thereof, provided that J₂ is also present;

J₂ is absent or is a charged amino acid residue, typically a basic amino acid residue, e.g., Lys, or modified form thereof, provided that J₃ is also present;

J₃ is absent or is a charged amino acid residue, typically a basic amino acid residue, e.g., J₃ is selected from Lys or Arg, or modified form thereof, provided that J₄ is also present;

J₄ is absent or is selected from a small amino acid residue, e.g., T, or modified form thereof, or a charged amino acid residue, typically a basic amino acid residue, e.g., J₄ is selected from Lys or Arg, or modified form thereof, or a neutral/polar amino acid residue, e.g., Gin, or modified form thereof, provided that J₅ is also present; and

J₅ is absent or is selected from a small amino acid residue, e.g., J₅ is selected from Ala or Thr, or modified form thereof, or a hydrophobic amino acid residue, e.g., Leu, or modified form thereof;

In some embodiments, X₁ is selected from Ile, Val or Leu, or modified form thereof. In some embodiments, X₂ is selected from Thr, Gly, or Ala, or modified form thereof. In some embodiments, X₃ is selected from Ile or Leu, or modified form thereof. In some embodiments, X₄ is a hydrophobic amino acid residue, which is suitably selected from Val or Trp, or modified form thereof. In other embodiments, X₄ is a small amino acid residue, which is suitably selected from Ala, Ser or Thr, or modified form thereof. In some embodiments, X₅ is selected from Ile, Phe, or more typically Val, or modified form thereof.

In certain embodiments, [Xₗ]ₙ is represented by the formula:

J₆J₇ (X)

[SEQ ID NO:10]

wherein: at least one of J₆ and J₇ are present, in which

J₆ is selected from a hydrophobic amino acid residue, e.g., Leu, or modified form thereof, or a small amino acid residue, e.g., Gly, or modified form thereof; and

J₇ is selected from a small amino acid residue, e.g., Ser, or modified form thereof, or a hydrophobic amino acid residue, e.g., Leu, or modified form thereof.

In some embodiments, X₆ is a small amino acid residue, which is suitably Ala, or modified form thereof. In other embodiments, X₆ is a hydrophobic amino acid residue, which is suitably selected from Val or Leu, or modified form thereof. In some embodiments, X₇ is a small amino acid residue, which is suitably selected from Ala, Gly or Thr, or modified form thereof. In other embodiments, X₇ is Leu, or modified form thereof. In some embodiments, X₈ is a hydrophobic amino acid residue, which is suitably selected from Leu or Val, or modified form thereof. In other embodiments, X₈ is a small amino acid residue, which is suitably selected from Ala or Ser, or modified form thereof. In some embodiments, X₉ is a hydrophobic amino acid residue, which is suitably selected from Val or Leu, or modified form thereof. In other embodiments, X₉ is a small amino acid residue, which is suitably selected from Ala or Gly, or modified form. In some embodiments, X₁₀ is Gln or modified form thereof. In other embodiments, X₁₀ is a hydrophobic amino acid residue, which is suitably selected from Ile, Val or Phe, or modified form.

In some embodiments, X₁₁ is a small amino acid residue, which is suitably selected from Pro, Ala or Thr or modified form thereof. In other embodiments, X₁₁ is Phe or modified form thereof. In still other embodiments, X₁₁ is Gln, or modified form thereof. In some embodiments, X₁₂ is a small amino acid residue, which is suitably selected from Ala, Ser or Thr, or modified form thereof. In some embodiments, X₁₃ is a hydrophobic amino acid residue, which is suitably selected from Val, Ile or Met, or modified form thereof. In other embodiments, X₁₃ is a small amino acid residue, e.g., Ala or modified form thereof. In some embodiments, X₁₄ is selected from Pro or Ala, or modified form thereof. In some embodiments, X₁₅ is a neutral/polar amino acid residue, e.g., Gin, or modified form thereof. In other embodiments, X₁₅ is an acidic amino acid residue, e.g., Asp, or modified form thereof. In still other embodiments, X₁₅ is a hydrophobic amino acid residue, e.g., Leu, or modified form thereof. In some embodiments, X₁₆ is Ala, or modified form thereof.

In certain embodiments, Z₁ is represented by the formula:

J₈J₉J₁₀ (XI)

[SEQ ID NO:11]

wherein: J₈ is a small amino acid residue, e.g., Thr, or modified form thereof;

J₉ is absent or is a charged amino acid residue, typically a basic amino acid residue, e.g., Lys, or modified form thereof, provided that J₈ is also present; and

J₁₀ is absent or is a charged amino acid residue, typically a basic amino acid residue, e.g., Lys, or modified form thereof, provided that J₉ is also present.

The amino acids in the SCE may be those encoded by genes or analogues thereof or the D-isomers thereof. Compounds within the scope of the present invention can be obtained by modifying the disclosed formulae in numerous ways, while preserving the activity of the SCE thus obtained. For example, while the amino acids of these compounds are normally in the natural L optical isomer form, one or more, usually two or less and preferably one amino acid may be replaced with the optical isomer D form, or a D,L-racemic mixture can be provided in the molecules comprising the SCE. In one embodiment, the SCE is in a form wherein all of the residues are in the D-configuration thus conferring resistance to protease activity while retaining self-coalescing properties. The resulting molecules are themselves enantiomers of the native L-amino acid-containing forms.

The nomenclature used to describe SCEs follows the conventional practice wherein the amino group is presented to the left and the carboxy group to the right of each amino acid residue. In the formulae representing selected specific embodiments of the present invention, the amino-(N-) and carboxy-(C-) terminal groups, although not specifically shown, will be understood to be in the form they would assume at physiological pH values, unless otherwise specified. In the amino acid structure formulae, each residue is generally represented by a single letter designation, corresponding to the trivial name of the amino acid, in accordance with the following table, in which the three-letter designations for each residue is also shown:

TABLE B
Abbreviations for amino acids

| | | Three- | | One- | Three- |
|---|---|---|---|---|---|
| | One-Letter | Letter | | Letter | Letter |
| Amino Acid | Symbol | Symbol | Amino Acid | Symbol | Symbol |
| Alanine | A | Ala | Leucine | L | Leu |
| Arginine | R | Arg | Lysine | K | Lys |
| Asparagine | N | Asn | Methionine | M | Met |
| Aspartic acid | D | Asp | Phenylalanine | F | Phe |
| Cysteine | C | Cys | Proline | P | Pro |
| Glutamine | Q | Gln | Serine | S | Ser |
| Glutamic acid | E | Glu | Threonine | T | Thr |
| Glycine | G | His | Tryptophan | W | Trp |
| Histidine | H | Ile | Tyrosine | Y | Tyr |
| Isoleucine | I | | Valine | V | Val |

The SCEs of the present invention are peptides or peptide-like compounds which are partially defined in terms of amino acid residues of designated classes. Amino acid residues can be generally sub-classified into major subclasses as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterises certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al. (1978) A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., 1992, Science 256(5062): 144301445), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always nonaromatic. Dependent on their structural properties, amino acid residues may fall in two or more classes.

For the naturally-occurring protein amino acids, sub-classification according to the foregoing scheme is presented in the following table.

TABLE C
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

The “modified” amino acids that may be included in the SLEs are gene-encoded amino acids which have been processed after translation of the gene, e.g., by the addition of methyl groups or derivatization through covalent linkage to other substituents or oxidation or reduction or other covalent modification. The classification into which the resulting modified amino acid falls will be determined by the characteristics of the modified form. For example, if lysine were modified by acylating the ε-amino group, the modified form would not be classed as basic but as polar/large.

Certain commonly encountered amino acids, which are not encoded by the genetic code, include, for example, β-alanine (β-Ala), or other omega-amino acids, such as 3-aminopropionic, 2,3-diaminopropionic (2,3-diaP), 4-aminobutyric and so forth, α-aminoisobutyric acid (Aib), sarcosine (Sar), ornithine (Orn), citrulline (Cit), t-butylalanine (t-BuA), t-butylglycine (t-BuG), N-methylisoleucine (N-MeIle), phenylglycine (Phg), and cyclohexylalanine (Cha), norleucine (Me), 2-naphthylalanine (2-Nal); 1,2,3,4-tetrahydroisoquinoline-3-carboxylic acid (Tic); β-2-thienylalanine (Thi); methionine sulfoxide (MSO); and homoarginirie (Har). These also fall conveniently into particular categories.

Based on the above definitions, Sar, β-Ala and Aib are small, t-BuA, t-BuG, N-MeIle, Nle, Mvl, Cha, Phg, Nal, Thi and Tic are hydrophobic; 2,3-diaP, Orn and Har are basic; Cit, Acetyl Lys and MSO are neutral/polar/large. The various omega-amino acids are classified according to size as small (β-Ala and 3-aminopropionic) or as large and hydrophobic (all others).

Other amino acid substitutions for those encoded in the gene can also be included in SCEs within the scope of the invention and can be classified within this general scheme according to their structure.

In the SCEs of the invention, one or more amide linkages (—CO—NH—) may optionally be replaced with another linkage which is an isostere such as —CH₂NH—, —CH₂S—, —CH₂CH₂, —CH CH— (cis and trans), COCH₂—, —CH(OH)CH₂— and —CH₂SO—. This replacement can be made by methods known in the art. The following references describe preparation of peptide analogues which include these alternative-linking moieties: Spatola, A. F., Vega Data (March 1983), Vol. 1, Issue 3, “Peptide Backbone Modifications” (general review); Spatola, A. F., in “Chemistry and Biochemistry of Amino Acids Peptides and Proteins”, B. Weinstein, eds., Marcel Dekker, New York, p. 267 (1983) (general review); Morley, J. S., Trends Pharm Sci (1980) pp. 463468 (general review); Hudson, D., et al., Int J Pept Prot Res (1979) 14:177-185 (—CH₂NH—, —CH₂CH₂—); Spatola, A. F., et al., Life Sci (1986) 38:1243-1249 (—CH₂—S); Hann, M. M., J Chem Soc Perkin Trans I (1982) 307-314 (—CH—CH—, cis and trans); Almiquist, R. G., et al., J Med Chem (1980) 23:1392-1398 (—COCH₂—); Jennings-White, C., et al., Tetrahedron Lett (1982) 23:2533 (—COCH₂—); Szelke, M., et al., European Application EP 45665 (1982) CA:97:39405 (1982) (—CH(OH)CH₂—); Holladay, M. W., et al., Tetrahedron Lett (1983) 24:4401-4404 (—C(OH)CH₂—); and Hruby, V. J., Life Sci (1982) 31: 189-199 (—CH₂—S—).

Amino acid residues contained within the SCEs, and particularly at the carboxy- of amino-terminus, can also be modified by amidation, acetylation or substitution with other chemical groups which can, for example, change the solubility of the compounds without affecting their activity.

Exemplary SCE amino acid sequences include sequences of any naturally occurring membrane translocation sequence (MTS), which is typically but not exclusively selectable from naturally occurring signal sequences or variants thereof, that have the ability to aggregate into higher order aggregates under physiological conditions, such as inside of a cell. The naturally occurring MTS can be obtained from any suitable organism including, but not limited to, bacteria, mycobacteria, viruses, protozoa, yeast, plants and animals such as insects, avians, reptiles, fish and mammals. Suitably, the naturally occurring MTS is obtained from bacteria. Advantageously, the naturally occurring MTS amino acid sequence is selected from SEQ ID NO:12-90. In certain embodiments, the naturally occurring MTS amino acid sequence is selected from SEQ ID NO:67, 68, 69, 70, 71, 72, 73, 74, 75, 76, 77, 78, 79, 80, 81, 83, 84, 85 and 87.

In other embodiments, the SCE amino acid sequence includes the sequences of only that portion of an MTS responsible for the aggregation behavior. Thus, the present invention contemplates biologically active fragments of the MTS sequences of the invention. Persons skilled in the art will recognise that there are numerous techniques for producing such fragments. For example, a fragment of a reference MTS can be produced by amino and/or carboxyl terminal deletions as well as internal deletions, which can be obtained for example by enzymatic digestion. The fragment is then conjugated to a polypeptide of interest and the chimeric polypeptide so produced is then tested for the ability to form higher order aggregates. Such testing may employ an assay that provides a qualitative or quantitative determination of molecular weight including, but not restricted to, ultracentrifugation, electrophoresis (e.g., native polyacrylamide gel electrophoresis) and size separation (e.g., gel filtration, ultrafiltration). For example, higher order aggregation is tested by size exclusion chromatography as described in more detail below. In another embodiment, biological activity of an MTS fragment is tested by introducing into a cell a polynucleotide from which a chimeric polypeptide comprising an MTS fragment and a polypeptide of interest can be translated, and detecting the presence of higher order aggregates, which indicates that the fragment is a biologically active fragment.

Alternatively, an SCE, or its fragments, can differ from the corresponding sequence in SEQ ID NO:12-90. Thus, the present invention also contemplates variants of the naturally occurring or parent SCE amino acid sequences or their biologically-active fragments, wherein the variants are distinguished from the parent sequences by the addition, deletion, or substitution of one or more amino acids. In general, variants display at least 50%, 55%, 60%, 65%, 70%, 75%, 80%, 85%, 90%, 91%, 92%, 93%, 94%, 95%, 96%, 97%, 98%, 99% similarity to a parent SCE sequence as for example set forth in SEQ ID NO:12-90. Suitably, variants will have at least 50%, 55%, 60%, 65%, 70%, 75%, 80%, 85%, 90%, 91%, 92%, 93%, 94%, 95%, 96%, 97%, 98%, 99% sequence identity to a parent SCE sequence as for example set forth in any one of SEQ ID NO:12-90. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, or more amino acids but which retain the self-coalescing properties and the ability to confer higher order aggregation to a molecule of interest, are contemplated. Polypeptides of the invention include polypeptides that are encoded by polynucleotides that hybridise under stringent, preferably highly stringent conditions to the polynucleotide sequences of the invention, or the non-coding strand thereof, as described infra. In one embodiment, it differs by at least one but by less than 15, 10, 8, 6, 5, 4, 3, 2 or 1 amino acid residues. In another, it differs from the corresponding sequence in SEQ ID NO:12-90 by at least one residue but less than 20%, 15%, 10% or 5% of the residues. (If this comparison requires alignment the sequences should be aligned for maximum similarity. “Looped” out sequences from deletions or insertions, or mismatches, are considered differences.) The differences are, suitably, differences or changes at a non-essential residue or a conservative substitution.

A “non-essential” amino acid residue is a residue that can be altered from the wild-type sequence of an SCE without abolishing or substantially altering its self-coalescing activity. Suitably, the alteration does not substantially alter the self-coalescing activity, e.g., the activity is at least 20%, 40%, 60%, 70% or 80% of wild-type. An “essential” amino acid residue is a residue that, when altered from the wild-type sequence of an SCE, results in abolition of the self-coalescing activity such that less than 20% of the wild-type activity is present. From a review of the sequence comparisons of SCEs shown in FIGS. 1 and 2, it is clear that none of the amino acid residues of SCEs is absolutely conserved across the SCEs presented in those figures. Accordingly, it is believed that all amino acid residues of the SCEs are amenable to alteration, especially to conservative amino acid substitution.

A “conservative amino acid substitution” is one in which the amino acid residue is replaced with an amino acid residue having a similar side chain. Families of amino acid residues having similar side chains have been defined in the art and certain subclasses are described above in Table C. Preferred variant SCEs are those having conserved amino acid substitutions. Examples of conservative substitutions include the following: aspartic-glutamic as acidic amino acids; lysine/arginine/histidine as basic amino acids; serine/glycine/alanine/threonine as small amino acids; leucine/isoleucine, methionine/valine, alanine/valine as hydrophobic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional SCE can readily be determined by assaying the specific coalescing or aggregating activity of the variant SCE. Conservative substitutions are shown in Table D below under the heading of exemplary substitutions. More preferred substitutions are shown under the heading of preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE D
EXEMPLARY AND PREFERRED AMINO ACID SUBSTITUTIONS

| Original | | Preferred |
|---|---|---|
| Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm. C. Brown Publishers (1993).

Thus, a predicted non-essential amino acid residue in an SCE is typically replaced with another amino acid residue from the same side chain family. Alternatively, in another embodiment, mutations can be introduced randomly along all or part of an SCE coding sequence, such as by saturation mutagenesis, and the resultant mutants can be screened for self-coalescing activity to identify mutants that retain activity. Following mutagenesis of such coding sequences, the encoded peptide can be expressed recombinantly and the activity of the peptide can be determined.

In other embodiments, the SCE includes an amino acid sequence having at least about 50%, 55%, 60%, 65%, 70%, 75%, 80%, 85%, 90%, 91%, 92%, 93%, 94% 95%, 96%, 97%, 98% or more similarity to a corresponding sequence of SEQ ID NO:12-90, and has self-coalescing activity.

The SCEs of the invention contain a significant number of structural characteristics in common with each other as for example depicted in FIGS. 1 and 2. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally-occurring and can be from either the same or different species. Members of a family can also have common functional characteristics.

Variant SCE sequences, which differ from a parent SCE sequence, by the substitution, addition or deletion of at least one amino acid residue may be synthesised de novo using solution or solid phase peptide synthesis techniques as known in the art. Alternatively such variants, including variants of naturally-occurring SCE sequences may be conveniently obtained by mutagenesis of their coding sequences. Mutations in nucleotide sequences constructed for expression of variants must, of course, preserve the reading frame phase of the coding sequences and suitably will not create complementary regions that could hybridise to produce secondary mRNA structures such as loops or hairpins which would adversely affect translation of the mRNA. Although a mutation site may be predetermined, it is not necessary that the nature of the mutation per se be predetermined. For example, in order to select for optimum characteristics of mutants at a given site, random mutagenesis may be conducted at the target codon and the expressed mutants screened for coalescent activity.

In one embodiment, mutations can be introduced at particular loci by synthesising oligonucleotides encoding the desired amino acid residues, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Exemplary methods of making the alterations set forth above are disclosed by Walder et al. (1986, Gene 42:133); Bauer et al. (1985, Gene 37:73); Craik (1985, Bio Techniques January 12-19,); Smith et al. (Genetic Engineering: Principles and Methods, Plenum Press, 1981); and U.S. Pat. Nos. 4,518,584 and 4,737,462.

In another variation of the invention, an SCE amino acid sequence of the invention is encoded by a polynucleotide that hybridises to a nucleotide sequence encoding an SCE amino acid sequence as set forth in SEQ ID NO:12-90; or the non-coding strands complementary to these sequences, under stringency conditions described herein. In a preferred embodiment, the SCE amino acid sequence is encoded by a polynucleotide that hybridises to a nucleotide sequence as set forth in SEQ ID NO:91-132 under a stringency condition described herein. As used herein, the term “hybridises under low stringency, medium stringency, high stringency, or very high stringency conditions” describes conditions for hybridisation and washing. Guidance for performing hybridisation reactions can be found in Ausubel et al., (1998, supra), Sections 6.3.1-6.3.6. Aqueous and non-aqueous methods are described in that reference and either can be used.

In one embodiment, the present invention contemplates polynucleotides which hybridise to a reference polynucleotide encoding an SCE amino acid sequence of the invention under at least low stringency conditions. Reference herein to low stringency conditions include and encompass from at least about 1% v/v to at least about 15% v/v formamide and from at least about 1 M to at least about 2 M salt for hybridisation at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridisation in 6×sodium chloride/sodium citrate (SSC) at about 45° C., followed by two washes in 0.2×SSC, 0.1% SDS at least at 50° C. (the temperature of the washes can be increased to 55° C. for low stringency conditions).

In another embodiment, the present invention contemplates polynucleotides which hybridise to a reference SCE-encoding polynucleotide under at least medium stringency conditions. Medium stringency conditions include and encompass from at least about 16% v/v to at least about 30% v/v formamide and from at least about 0.5 M to at least about 0.9 M salt for hybridisation at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridising in 6×SSC at about 45° C., followed by one or more washes in 0.2×SSC, 0.1% SDS at 60° C.

In another embodiment, the present invention contemplates polynucleotides which hybridise to a reference SCE-encoding polynucleotide under high stringency conditions. High stringency conditions include and encompass from at least about 31% v/v to at least about 50% v/v formamide and from about 0.01 M to about 0.15 M salt for hybridisation at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridisation at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridising in 6×SSC at about 45° C., followed by one or more washes in 0.2×SSC, 0.1% SDS at 65° C.

In certain embodiments, an isolated nucleic acid molecule of the invention hybridises under very high stringency conditions. One embodiment of very high stringency conditions includes hybridising 0.5M sodium phosphate, 7% SDS at 65° C., followed by one or more washes at 0.2×SSC, 1% SDS at 65° C.

Other stringency conditions are well known in the art and a skilled addressee will recognise that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridisation rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:

Tₘ=81.5+16.6(log₁₀M)+0.41(%G+C)−0.63(% formamide)−(600/length)

wherein: M is the concentration of Na₊, preferably in the range of 0.01 molar to 0.4 molar; %G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ −15° C. for high stringency, or Tₘ −30° C. for moderate stringency.

In a preferred hybridisation procedure, a membrane (e.g., a nitrocellulose membrane or a nylon membrane) containing immobilised DNA is hybridised overnight at 42° C. in a hybridisation buffer (50% deionised formamide, 5×SSC, 5×Denhardt's solution (0.1% ficoll, 0.1% polyvinylpyrollidone and 0.1% bovine serum albumin), 0.1% SDS and 200 mg/mL denatured salmon sperm DNA) containing labelled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1%SD solution for 12 min at 65-68° C.

Also provided are isolated polynucleotides comprising a nucleotide sequence that encodes at least one SCE amino acid sequence, wherein the SCE-encoding portion of the polynucleotide is at least about 99%, at least about 98%, at least about 95%, at least about 90%, at least about 85%, at least about 80%, at least about 75%, or at least about 70% identical over its full length to a reference SCE-encoding polynucleotide as for example set forth in SEQ ID NO:91-132. Natural or artificial sequences can be screened for SCE properties by any suitable method known to persons of skill in the art. For example, one may test a natural or artificial sequence for the capacity to form higher order aggregates by conjugating the sequence to a polypeptide of interest and then testing the chimeric polypeptide so produced in an assay that provides a qualitative or quantitative determination of molecular weight for the ability to form higher order aggregates. Suitably, higher order aggregation of such chimeric molecules is tested by size exclusion chromatography as described in more detail below.

2.2 Molecules of Interest

A molecule of interest may be selected from any compound including organic and inorganic compounds. In certain embodiments, the molecule of interest is selected from organic compounds including, but not limited to, drugs (e.g. antibiotics, hormones, and drugs for treating conditions such as cancer, diabetes, inflammation, cardiovascular disease, sexual dysfunction, neuropsychiatric disorders and the like), metabolites and agrochemical compounds such as pesticides and herbicides. Typically, the molecule of interest is an organic polymer and desirably a polymer of biological origin such as a polynucleotide or polypeptide. In one embodiment of this type, the molecule of interest is a polypeptide having an enzymatic, therapeutic or antigenic activity. Thus, in this embodiment, the chimeric molecule is a chimeric polypeptide comprising an SCE that is fused, linked or otherwise associated to a “polypeptide of interest”. By “chimeric polypeptide” is meant a polypeptide comprising at least two distinct polypeptide segments (domains) that do not naturally occur together as a single protein. In preferred embodiments, each domain contributes a distinct and useful property to the polypeptide. Polynucleotides that encode chimeric polypeptides can be constructed using conventional recombinant DNA technology to synthesise, amplify, and/or isolate polynucleotides encoding the at least two distinct segments, and to ligate them together. See, e.g., Sambrook et al., Molecular Cloning—A Laboratory Manual, Second Ed., Cold Spring Harbor Press (1989); and Ausubel et al., Current Protocols in Molecular Biology, John Wiley & Sons, Inc. (1998).

A polypeptide of interest may be selected from any polypeptide that is of commercial or practical interest and that comprises an amino acid sequence, which is typically but not exclusively encodable by the codons of the universal genetic code. Exemplary polypeptides of interest include: enzymes that may have utility in chemical (e.g., enzymes for selective hydrolysis of cyclic secondary alcohols or for transesterification of activated/nonactivated esters), food-processing (e.g., amylases), or other commercial applications (detergent enzymes); enzymes having utility in biotechnology applications, including DNA and RNA polymerases, endonucleases, exonucleases, peptidases, and other DNA and protein modifying enzymes; polypeptides that are capable of specifically binding to compositions of interest, such as polypeptides that act as intracellular or cell surface receptors for other polypeptides, for steroids, for carbohydrates, or for other biological molecules; polypeptides that comprise at least one antigen-binding domain of an antigen-binding molecule; polypeptides that comprise the ligand-binding domain of a ligand-binding protein (e.g., the ligand binding domain of a cell surface receptor); metal binding proteins (e.g., ferritin (apoferritin), metallothioneins, and other metalloproteins), which are useful for isolating/purifying metals from a solution containing them for metal recovery or for remediation of the solution; light-harvesting proteins (e.g., proteins used in photosynthesis that bind pigments); proteins that can spectrally alter light (e.g., light spectrum-modifying polypeptides that absorb light at one wavelength and emit light at another wavelength); regulatory proteins, such as transcription factors and translation factors; and polypeptides of therapeutic value, such as chemokines, cytokines, interleukins, growth factors, interferons, metabolic polypeptides, immunopotentiators and iummunosuppressors, angiogenic or anti-angiogenic peptides and antigens.

In some embodiments, the polypeptide of interest is selected from cytokines, growth factors, and hormones, which include, but are not limited to: interferon-α, interferon-β, interferon-γ, interleukin-1, interleukin-2, interleukin-3, interleukin4, interleukin-5, interleukin-6, interleukin-7, interleukin-8, interleukin-9, interleukin-10, interleukin-11, interleukin-12, interleukin-13, interleukin-14, interleukin-15, interleukin-16, erythropoietin, colony-stimulating factor-1, granulocyte colony-stimulating factor, granulocyte-macrophage colony-stimulating factor, leukemia inhibitory factor, tumor necrosis factor, lymphotoxin, platelet-derived growth factor, fibroblast growth factors, vascular endothelial cell growth factor, epidermal growth factor, transforming growth factor-β, transforming growth factor-α, thrombopoietin, stem cell factor, oncostatin M, amphiregulin, Mullerian-inhibiting substance, B-cell growth factor, macrophage migration inhibiting factor, monocyte chemoattractant protein (e.g., MCP-1), endostatin, and angiostatin, as well as their agonists and antagonists.

In some embodiments, the polypeptide of interest is an antigen which can be selected from any foreign or autologous antigens including, but not restricted to, viral, bacterial, protozoan, microbial, tumor antigens as well as self- or auto-antigens. Suitable viral antigens are derived from human immunodeficiency virus (HIV), papilloma virus poliovirus, and influenza virus, Rous sarcoma virus or a virus causing encephalitis such as Japanese encephalitis virus, a herpesvirus including, but not limited to, herpes simplex virus and Epstein-Barr virus, cytomegalovirus, a parvovirus, or a hepatitis virus including, but not limited to, hepatitis strains A, B and C. Desirable bacterial antigens include, but are not limited to, those derived from Neisseria species, Meningococcal species, Haemophilus species Salmonella species, Streptococcal species, Legionella species and Mycobacterium species. Suitable protozoan antigens include, but are not restricted to, those derived from Plasmodium species, Schistosoma species, Leishmania species, Trypanosoma species, Toxoplasma species and Giardia species. Any cancer or tumor antigen is contemplated by the present invention. For example, such antigen may be derived from, melanoma, lung cancer, breast cancer, cervical cancer, prostate cancer, colon cancer, pancreatic cancer, stomach cancer, bladder cancer, kidney cancer, post transplant lymphoproliferative disease (PTLD), Hodgkin's Lymphoma and the like.

In some embodiments, the polypeptide of interest is a metabolic polypeptide, including polypeptides involved in biotransformation of compounds, such as but not limited to, absorption, binding, uptake, excretion, distribution, transport, processing, conversion or degradation of compounds. For example, metabolic polypeptides include, but are not limited to, drug-metabolising polypeptides (e.g., cytochrome p450 (CYP) isoforms, esterases, acetyl-transferases, acetylases, glucuronosyl-transferases, glucuronidases, glutathione S-transferases and the like), drug-binding polypeptides (e.g., serum albumin, α-acidic glycoprotein and the like), ornithine transcarbamylase, arginosuccinate synthetase, glutamine synthetase, glycogen synthetase, glucose-6-phosphatase, succinate dehydrogenase, glucokinase, insulin, pyruvate kinase, acetyl CoA carboxylase, fatty acid synthetase, alanine aminotransferase, glutamate dehydrogenase, ferritin, low density lipoprotein (LDL) receptor, P450 enzymes, or alcohol dehydrogenase.

In other embodiments, the molecule of interest is a peptide, which is suitably selected from antigenic peptides (including T cell epitopes, B cell epitopes), peptides derived from cytokines, which have a cytokine activity, peptides derived from chemokines, which have a chemokine activity, neuropeptides, anti-inflammatory peptides and receptor ligand peptides, which can block receptor function in aggregate form.

In still other embodiments, the molecule of interest is a hormone, which includes trace substances produced by various endocrine glands which serve as chemical messengers carried by biological fluids including blood to various target organs, where they regulate a variety of physiological and metabolic activities in vertebrates. Suitable hormones include growth hormones, sex hormones, thyroid hormones, pituitary hormones and melanocyte stimulating hormones. For example, the hormone may be selected from estrogens (e.g., estradiol, estrone, estriol, diethylstibestrol, quinestrol, chlorotrianisene, ethinyl estradiol, mestranol), anti-estrogens (such as, for example, clomiphene, tamoxifen), progestins (e.g., medroxyprogesterone, norethindrone, hydroxyprogesterone, norgestrel), antiprogestin (e.g., mifepristone), androgens (e.g., testosterone, testosterone cypionate, dihydrotestosterone, fluoxymesterone, danazol, testolactone), anti-androgens (e.g., cyproterone acetate, flutamide) and the like. Alternatively, the hormone may be selected from thyroid hormones (e.g., triiodothyronne, thyroxine, propylthiouracil, methimazole, and iodixode) and gastrointestinal hormones (e.g., gastrin, glucagon, secretin, cholecystokinin, gastric inhibitory peptide, vasoactive intestinal peptide, substance P, glucagon-like immunoreactivity peptide, somatostatin, bombesin, neurotensin and the like). The hormone may also be selected from pituitary hormones (e.g., corticotropin, sumutotropin, oxytocin, and vasopressin) and hormones of the adrenal cortex (e.g., adrenocorticotropic hormone (ACTH), aldosterone, cortisol, corticosterone, deoxycorticosterone and dehydroepiandrosterone). Other hormones include prednisone, betamethasone, vetamethasone, cortisone, dexamethasone, flunisolide, hydrocortisone, methylprednisolone, paramethasone acetate, prednisolone, triamcinolone fludrocortisone and the like.

In still other embodiments, the molecule of interest is linked to or otherwise associated with an ancillary molecule, which comprises a different activity than the molecule of interest. In some embodiments, the activity of the ancillary molecule ameliorates or otherwise reduces an unwanted activity (or side effect) of the molecule of interest. For example, the ancillary molecule may be an immunostimulatory molecule, as for example disclosed in U.S. Pat. No. 6,228,373 and U.S. Pat. No. 5,466,669, or an immunosuppressive molecule, as for example disclosed in U.S. Pat. No. 5,679,640, which enhances or reduces, respectively, the capacity of the molecule(s) of interest, when in aggregate form, to produce an antigen-specific immune response to the molecule of interest in an animal to which the aggregate has been administered.

3. Methods of Producing Chimeric Molecules of the Invention

Chimeric molecules comprising an SCE and a molecule of interest can be produced by any suitable technique known to persons of skill in the art. The present invention, therefore, is not dependent on, and not directed to, any one particular technique for conjugating an SCE with a molecule of interest.

The manner of attachment of the SCE to a molecule of interest should be such that the self-coalescing property of the SCE is not impaired and also such that, on self-assembly of the chimeric molecule into a higher order aggregate the molecule of interest is exposed to the exterior of the aggregate, allowing for interaction of that molecule with a cognate binding or interacting partner molecule. A linker or spacer may be included between the SCE and the molecule of interest to spatially separate the SCE from the molecule of interest. The linker or spacer molecule may be from about 1 to about 100 atoms in length. In some embodiments, the linker or spacer molecule comprises one or more amino acid residues (e.g., from about 1 to about 50 amino acid residues and desirably 1, 2, 3, 4, 5, 6, 7, 8, 9, 10, 15, 20 amino acid residues). Such linkers or spacers may facilitate the proper folding of the molecule of interest, to assure that it retains a desired activity even when the chimeric molecule as a whole has formed aggregates with other chimeric SCE-containing molecules. The SCE and the molecule of interest may be in either order i.e., the molecule of interest may be conjugated to the amino-terminus or the carboxyl-terminus of the SCE.

Suitably, the molecule of interest is covalently attached to the SCE. Covalent attachment may be achieved by any suitable means known to persons of skill in the art. For example, a chimeric polypeptide may be prepared by linking polypeptides together using crosslinking reagents. Examples of such crosslinking agents include carbodiimides such as but not limited to 1-cyclohexyl-3-(2-morpholinyl-(4-ethyl)carbodiimide (CMC), 1-ethyl-3-(3-dimethyaminopropyl)carbodiimide (EDC) and 1-ethyl-3-(4-azonia-4,4-dimethylpentyl) carbodiimide. Exemplary crosslinking agents of this type are selected from the group consisting of 1-cyclohexyl-3-(2-morpholinyl-(4-ethyl)carbodiimide,(1-ethyl-3-(3-dimethya minopropyl carbodiimide (EDC) and 1-ethyl-3-(4-azonia-4,4-dimethylpentyl)carbodiimide. Examples of other suitable crosslinking agents are cyanogen bromide, glutaraldehyde and succinic anhydride.

In general any of a number of homobifunctional agents including a homobifunctional aldehyde, a homobifunctional epoxide, a homobifunctional imidoester, a homobifunctional N-hydroxysuccinimide ester, a homobifunctional maleimide, a homobifunctional alkyl halide, a homobifunctional pyridyl disulfide, a homobifunctional aryl halide, a homobifunctional hydrazide, a homobifunctional diazonium derivative and a homobifunctional photoreactive compound may be used. Also included are heterobifunctional compounds, for example, compounds having an amine-reactive and a sulfhydryl-reactive group, compounds with an amine-reactive and a photoreactive group and compounds with a carbonyl-reactive and a sulfhydryl-reactive group.

Homobifunctional reagents are molecules with at least two identical functional groups. The functional groups of the reagent generally react with one of the functional groups on a protein, typically an amino group. Specific examples of such homobifunctional crosslinking reagents include the bifunctional N-hydroxysuccinimide esters dithiobis(succinimidylpropionate), disuccinimidyl suberate, and disuccinimidyl tartrate; the bifunctional imidoesters dimethyl adipimidate, dimethyl pimelimidate, and dimethyl suberimidate; the bifunctional sulfhydryl-reactive crosslinkers 1,4-di-[3′-(2′-pyridyldithio)propionamido]butane, bismaleimidohexane, and bis-N-maleimido-1,8-octane; the bifunctional aryl halides 1,5-difluoro-2,4-dinitrobenzene and 4.4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether, the bifunctional hydrazides adipic acid dihydrazide, carbohydrazide, and succinic acid dihydrazide; the bifunctional diazoniums o-toluidine, diazotized and bis-diazotized benzidine; the bifunctional alkylhalides N,N′-ethylene-bis(iodoacetamide), N,N′-hexamethylene-bis(iodoacetamide), N,N′-undecamethylene-bis(iodoacetamide), as well as benzylhalides and halomustards, such as .alpha.,.alpha.′-diiodo-p-xylene sulfonic acid and tri(2-chloroethyl)amine, respectively. Methods of using homobifunctional crosslinking reagents are known to practitioners in the art. For instance, the use of glutaraldehyde as a cross-linking agent is described for example by Poznansky et al. (1984, Science, 223: 1304-1306). The use of diimidates as a cross-linking agent is described for example by Wang, et al. (1977, Biochemistry, 16: 2937-2941).

Although it is possible to use homobifunctional crosslinking reagents for the purpose of forming a chimeric polypeptide according to the invention, skilled practitioners in the art will appreciate that it is more difficult to attach different proteins in an ordered fashion with these reagents. In this regard, in attempting to link a first protein with a second protein by means of a homobifunctional reagent, one cannot prevent the linking of the first protein to each other and of the second to each other. Accordingly, heterobifunctional crosslinking reagents are preferred because one can control the sequence of reactions, and combine proteins at will. Heterobifunctional reagents thus provide a more sophisticated method for linking two polypeptide. These reagents require one of the molecules to be joined, hereafter called Partner B, to possess a reactive group not found on the other, hereafter called Partner A, or else require that one of the two functional groups be blocked or otherwise, greatly reduced in reactivity while the other group is reacted with Partner A. In a typical two-step process for forming heteroconjugates, Partner A is reacted with the heterobifunctional reagent to form a derivatised Partner A molecule. If the unreacted functional group of the crosslinker is blocked, it is then deprotected. After deprotecting, Partner B is coupled to derivatised Partner A to form the conjugate. Primary amino groups on Partner A are reacted with an activated carboxylate or imidate group on the crosslinker in the derivatisation step. A reactive thiol or a blocked and activated thiol at the other end of the crosslinker is reacted with an electrophilic group or with a reactive thiol, respectively, on Partner (B. When the crosslinker possesses a reactive thiol, the electrophile on Partner B preferably will be a blocked and activated thiol, a maleimide, or a halomethylene carbonyl (eg. bromoacetyl or iodoacetyl) group. Because biological macromolecules do not naturally contain such electrophiles, they must be added to Partner B by a separate derivatisation reaction. When the crosslinker possesses a blocked and activated thiol, the thiol on Partner B with which it reacts may be native to Partner B.

An example of a heterobifunctional reagent is N-succinimidyl 3-(2-pyridyldithio)propionate (SPDP) (see for example Carlsson et al., 1978, Biochem. J., 173: 723-737). Other heterobifunctional reagents for linking proteins include for example succinimidyl 4-(N-maleimidomethyl)cyclohexane-1-carboxylate (SMCC) (Yoshitake et al., 1979, Eur. J. Biochem, 101: 395-399), 2-iminothiolane (IT) (Jue et al., 1978, Biochemistry, 17: 5399-5406), and S-acetyl mercaptosuccinic anhydride (SAMSA) (Klotz and Heiney, 1962, Arch. Biochem. Biophys., 96: 605-612). All three react preferentially with primary amines (eg. lysine side chains) to form an amide or amidine group which links a thiol to the derivatized molecule (eg. a heterologous antigen) via a connecting short spacer arm, one to three carbon atoms long.

Another example of a heterobifunctional reagent is N-succinimidyl 3-(2-pyridyldithio)butyrate (SPDB) (Worrell et al., 1986, Anti-Cancer Drug Design, 1: 179-188), which is identical in structure to SPDP except that it contain a single methyl-group branch alpha to the sulfur atom which is blocked and activated by 2-thiopyridine. SMPT and SMBT described by Thorpe et al. (1987, Cancer Research, 47: 5924-5931) contain a phenylmethyl spacer arm between an N-hydroxysuccinimide-activated carboxyl group and the blocked thiol; both the thiol and a single methyl-group branch are attached to the aliphatic carbon of the spacer arm. These heterobifunctional reagents result in less easily cleaved disulfide bonds than do unbranched crosslinkers.

Some other examples of heterobifunctional reagents containing reactive disulfide bonds include sodium S-4-succinimidyloxycarbonyl-α-methylbenzylthiosulfate, 4-succinimidyl-oxycarbony-α-methyl-(2-pyridyldithio)toluene.

Examples of heterobifunctional reagents comprising reactive groups having a double bond that reacts with a thiol group include SMCC mentioned above, succinimidyl m-maleimidobenzoate, succinimidyl 3-(maleimido)propionate, sulfosuccinimidyl 4-(p-maleimidophenyl)butyrate, sulfosuccinimidyl 4-(N-maleimidomethylcyclohexane-1-carboxylate and maleimidobenzoyl-N-hydroxysuccinimide ester (MBS). In a preferred embodiment, MBS is used to produce the conjugate.

Other heterobifunctional reagents for forming conjugates of two proteins are described for example by Rodwell et al. in U.S. Pat. No. 4,671,958 and by Moreland et al. in U.S. Pat. No. 5,241,078.

Crosslinking of the SCE and the molecule of interest may be accomplished by coupling a carbonyl group to an amine group or to a hydrazide group by reductive amination.

Alternatively, chimeric polypeptides may be synthesised using solution synthesis or solid phase synthesis as described, for example, in Chapter 9 of Atherton and Shephard (supra) and in Roberge et al (1995). Peptides of the present invention can be synthesised by solution or solid phase synthesis methods as known in the art. For example, the widely used Merrifield solid phase synthesis method, including the experimental procedures, is described in the following references: Stewart et al. (1969, Solid Phase Peptide Synthesis, W. H. Freeman Co., San Francisco); Merrifield (1963, J Am Chem Soc 85: 2149); Meienhofer (1973, Int J Pept Pro Res 11: 246); and Barany and Merrifield (1980, in The Peptides, E. Gross and F. Meinenhofer, eds., Vol. 2, Academic Press, pp. 3-285). The synthesis may use manual techniques or be completely automated, employing, for example, an Applied BioSystems 431A Peptide Synthesizer (Foster City, Calif.) or a Biosearch SAM II automatic peptide synthesizer (Biosearch, Inc., San Rafael, Calif.), following the instructions provided in the instruction manual and reagents supplied by the manufacturer. Disulphide bonds between Cys residues can be introduced by mild oxidation of the linear peptide by KCN as taught, for example, in U.S. Pat. No. 4,757,048 at Col. 20.

In another embodiment, the chimeric polypeptide is produced using recombinant nucleic acid based methodologies. Accordingly, another aspect of the present invention provides an isolated, synthetic or recombinant polynucleotide comprising a nucleotide sequence that encodes a chimeric polypeptide, wherein the polynucleotide comprises a first nucleotide sequence encoding at least one self-coalescing element (SCE) as broadly described above and fused in frame with a second nucleotide sequence encoding at least one polypeptide of interest. By, “in frame” is meant that when the polynucleotide is transformed into a host cell, the cell can transcribe and translate the polynucleotide sequence into a single polypeptide comprising both the SCE amino acid sequence and the at least one polypeptide of interest. For example, nucleic acid molecules encoding chimeric polypeptides can be synthesised de novo using readily available machinery. Sequential synthesis of DNA is described, for example, in U.S. Pat. No. 4,293,652. Instead of de novo synthesis, recombinant techniques may be employed including use of restriction endonucleases to cleave different SCE-encoding polynucleotides and use of ligases to ligate together in the same reading frame a cleaved polynucleotides encoding a molecule of interest. Suitable recombinant techniques are described for example in the relevant sections of Ausubel, et al. (supra) and of Sambrook, et al., (supra). Suitably, the synthetic polynucleotide is constructed using splicing by overlapping extension (SOEing) as for example described by Horton et al. (1990, Biotechniques 8(5): 528-535; 1995, Mol Biotechnol. 3(2): 93-99; and 1997, Methods Mol Biol. 67: 141-149). However, it should be noted that the present invention is not dependent on, and not directed to, any one particular technique for constructing the synthetic construct.

It is contemplated that the nucleotide sequences can be joined directly; or that the nucleotide sequences can be separated by additional codons. For example, additional codons also may be included between the sequence encoding the SCE amino acid sequence and the sequence encoding the at least one polypeptide of interest to provide a linker amino acid sequence that serves to spatially separate the SCE amino acid sequence from the polypeptide of interest. Such linkers may facilitate the proper folding of the polypeptide of interest, to assure that it retains a desired biological activity even when the chimeric polypeptide as a whole has formed aggregates with other chimeric polypeptides containing the SCE amino acid sequence. In some embodiments, especially when the polypeptide of interest does not comprise charged amino acids at, or closely adjacent to, its amino terminus, the linkers suitably comprise 1, 2, 3, 4, 5 or more charged, typically basic, amino acid residues that prevent or reduce the capacity of an SCE amino acid sequence to be cleaved intracellularly from the chimeric polypeptide. Desirably, these charged amino acid residues are placed at, or closely adjacent to, the amino terminus of the polypeptide of interest (e.g., within about 1, 2, 3, 4, 5 amino acid residues of the amino terminus). Also, additional codons may be included simply as a result of cloning techniques, such as ligations and restriction endonuclease digestions, and strategic introduction of restriction endonuclease recognition sequences into the polynucleotide.

The encoding sequences of the polynucleotide may be in either order i.e., the SCE amino acid encoding sequence may be upstream (5′) or downstream (3′) of the nucleotide sequence encoding the at least one polypeptide of interest, such that the SCE amino acid sequence of the resultant chimeric polypeptide is disposed at an amino-terminal or carboxyl-terminal position relative to the at least one polypeptide of interest. In a preferred embodiment, the nucleotide sequence encoding the SCE is disposed downstream (3′) of the sequence encoding the at least one polypeptide of interest. In an embodiment comprising sequences encoding two or more polypeptides of interest, the SCE-encoding sequence may be disposed between the two polypeptides of interest.

To the extent that such sequences are not already inherent in the above-described recombinant polynucleotides, it will be understood that such polynucleotides suitably further comprise regulatory elements such as but not limited to a translation initiation codon fused in frame and upstream (5′) of the encoding sequences, and a translation stop codon fused in frame and downstream (3′) of the encoding sequences. Such polynucleotides are useful for expression of a recombinant chimeric polypeptide in a suitable host cell. For example, a recombinant chimeric polypeptide according to the invention may be prepared by a procedure including the steps of (a) preparing a recombinant polynucleotide comprising a nucleotide sequence that encodes a chimeric polypeptide comprising a self-coalescing element fused with at least one polypeptide of interest, wherein the nucleotide sequence is operably linked to one or more regulatory elements; (b) introducing the recombinant polynucleotide into a suitable host cell; (c) culturing the host cell to express recombinant polypeptide from said recombinant polynucleotide; and (d) isolating the recombinant chimeric polypeptide from the cell or cell medium. Thus, also intended as part of the invention are vectors comprising the recombinant polynucleotides, and host cells comprising the polynucleotides or comprising the vectors. Vectors are useful for amplifying the polynucleotides in host cells. Preferred vectors include expression vectors, which contain appropriate regulatory elements to permit expression of the encoded chimeric protein in a host cell that has been transformed or transfect with the vectors. Expression vectors include, but are not limited to, self-replicating extra-chromosomal vectors such as plasmids, or vector that integrate into a host genome. The regulatory elements will generally be appropriate for the host cell used for expression. Numerous types of appropriate expression vectors and suitable regulatory sequences are known in the art for a variety of host cells. Typically, the regulatory elements include, but are not limited to, promoter sequences, leader or signal sequences, ribosomal binding sites, transcriptional initiation and termination sequences, translational initiation and termination sequences, and enhancer or activator sequences. Constitutive or inducible promoters as known in the art are contemplated by the invention. The promoters may be either naturally occurring promoters, or hybrid promoters that combine elements of more than one promoter.

In some embodiments, the expression vector contains a selectable marker gene to allow the selection of transformed or transfected host cells. Selection genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide. In order to express said fusion polypeptide, it is necessary to ligate a polynucleotide according to the invention into the expression vector so that the translational reading frames of the fusion partner and the polynucleotide coincide. Well known examples of fusion partners include, but are not limited to, glutathione-S-transferase (GST), Fc potion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG as described more fully hereinafter. Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localisation of the fusion polypeptide of the invention, or for isolating cells which express the fusion polypeptide of the invention. Flow cytometric methods such as fluorescence activated cell sorting (FACS) are particularly useful in this latter application. Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

In another embodiment, the polynucleotide includes 5′ and 3′ flanking regions that have substantial sequence homology with a region in the organism's genome, which can facilitate the introduction of the polynucleotide into the genome by homologous recombination.

The recombinant polynucleotide may be introduced into the host cell by any suitable method including transfection and transformation, the choice of which will be dependent on the host cell employed. Thus, another aspect of the present invention provides a host cell transformed or transfected with a recombinant polynucleotide encoding a chimeric polypeptide according to the invention. Such host cells are capable of producing a chimeric polypeptide of the invention, which can aggregate with other like chimeric polypeptides in vitro or in vivo, under conditions favorable to aggregation, to form higher order homo-aggregates. In an alternate embodiment, the invention contemplates a host cell transformed or transfected with at least two recombinant polynucleotides encoding chimeric polypeptides according to the invention, wherein the at least two polynucleotides encode compatible SCE amino acid sequences and distinct polypeptides of interest. Such host cells are capable of producing at least two chimeric polypeptides of the invention, which can aggregate with each other in vitro or in vivo, under conditions favorable to aggregation, to form higher ordered aggregates. Such hetero-aggregates can be used advantageously for example to provide a plurality of antigens for immunopotentiating a host against a disease or condition or to provide a plurality of enzymic activities for the catalysis of a multi-step chemical reaction. By “compatible” SCE amino acid sequences is meant SCE amino acid sequence that are either identical or sufficiently similar to permit co-aggregation with each other into higher order aggregates. Desirably, the two or more polypeptides of interest retain their native biological activity (e.g., antigenic activity, binding activity; enzymatic activity) in the higher order aggregate.

Suitable host cells for expression may be prokaryotic or eukaryotic. The host cell may be from the same kingdom (prokaryotic, animal, plant, fungi, protista, etc.) as the organism from which the SCE amino acid sequence of the polynucleotide was derived, or from a different kingdom. In a preferred embodiment, the host cell is from the same species as the organism from which the SCE amino acid sequence of the polynucleotide was derived. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

In yet another aspect, the invention contemplates a cell culture comprising host cells as broadly described above, wherein the cells express the chimeric polypeptide encoded by the polynucleotide as broadly described above, and wherein the cell culture includes cells wherein the chimeric polypeptide is present in the form of a higher order aggregate.

Recombinant chimeric polypeptides may be conveniently prepared by a person skilled in the art using standard protocols as for example described in Sambrook, et al., 1989, in particular Sections 16 and 17; Ausubel et al., (1994-1998), in particular Chapters 10 and 16; and Coligan et al., (1995-1997), in particular Chapters 1, 5 and 6. For example, such polypeptides may be prepared by culturing a host cell containing a recombinant polynucleotide as broadly described above. Thus, in another aspect, the invention contemplates a method for producing chimeric polypeptide as defined herein, comprising transforming or transfecting a cell with at least one recombinant polynucleotide of the invention; and growing the cell under conditions which result in expression of at least one chimeric polypeptide. In a preferred embodiment, the method further includes the step of isolating the chimeric polypeptide from the cell or from the growth medium of the cell.

The present invention also contemplates recombinant or synthetic chimeric polypeptides with or without associated native-protein glycosylation. Expression of recombinant polynucleotides as broadly described above in bacteria such as E. coli provides non-glycosylated molecules. Functional mutant variant chimeric polypeptides having inactivated N-glycosylation sites can be produced by oligonucleotide synthesis and ligation or by site-specific mutagenesis techniques. These variant polypeptides can be produced in a homogeneous, reduced carbohydrate form in good yield using yeast expression systems. N-glycosylation sites in eukaryotic proteins are characterised by the amino acid triplet Asn-A₁-Z, where A₁ is any amino acid except Pro, and Z is Ser or Thr. In this sequence, asparagine (Asn) provides a side chain amino group for covalent attachment of carbohydrate. Such a site can be eliminated by substituting another amino acid for Asn or for residue Z, deleting Asn or Z, or inserting a non-Z amino acid between A₁ and Z, or an amino acid other than Asn between Asn and A₁.

Recombinant chimeric polypeptides may also be prepared using genetically modified, typically non-human, animals. Accordingly, the present invention is directed towards genetically modified animals that express polynucleotides encoding the chimeric molecules of the invention. The genetic modification is generally in the form of a transgene and thus the genetically modified animal of the present invention is a transgenic animal that comprises at least one transgene in its cells, which includes a polynucleotide that encodes at least one chimeric molecule as broadly described above and that is operably linked to a regulatory element, which generally includes a transcriptional control element. The transgene is suitably contained within somatic cells of the animal, although it may also be contained within its germ cells. Usually, the transgenic animal is a mammal, which is suitably selected from the order Rodentia. In some embodiments, the transgenic mammal is a mouse, although rats are also of particular utility. However, it will be understood that the present invention is not restricted to these species. For example, the transgenic animal may be a goat, cow, sheep, dog, guinea pig or chicken.

The genetically modified animals of the present invention may be prepared by any number of means. In one method, a nucleic acid targeting construct or vector is prepared comprising two regions flanking the transgene wherein the regions are sufficiently homologous with portions of the genome of an animal to undergo homologous recombination with those portions. Alternatively, constructs for random integration need not include regions of homology to mediate recombination. Conveniently, markers for positive and negative selection are included in the constructs to permit selection of recombinant host cells. The targeting DNA construct is generally introduced into an embryonic stem (ES) cell or ES cell line. Methods for generating cells having gene modifications through homologous recombination are known in the art.

4. Production of Higher Order Aggregates

The invention also encompasses a method of producing a higher order aggregate. The method comprises providing a chimeric molecule comprising at least one SCE as herein defined, which is fused, linked or otherwise associated with a molecule of interest having a particular activity. The at least one SCE of the chimeric molecule is capable of coalescing with the SCEs of other chimeric molecules under conditions favorable to aggregation, whereby aggregation of the chimeric molecules results to form a higher order aggregate (i.e., homo-aggregate) with enhanced activity relative to the non-aggregated molecule of interest. In a preferred embodiment, the molecules of interest is a polypeptide and a higher-order homo-aggregate comprising the polypeptide is produced by expression of the chimeric molecules in a host cell under conditions favorable to aggregation.

In another embodiment, the invention provides a method of producing a higher order aggregate comprising two or more distinct activities. The method comprises providing at least two chimeric molecules, wherein an individual chimeric molecule comprises at least one SCE as herein defined, which is compatible with the SCE(s) of the other chimeric molecule(s), and which is fused, linked or otherwise associated with a molecule of interest having an activity distinct from the activity of other molecule(s) of interest corresponding to the other chimeric molecule(s). The SCEs of the first and second chimeric molecules will coalesce with each other under conditions favorable to aggregation so as to facilitate assembly of the chimeric molecules into higher order aggregates (i.e., hetero-aggregate) comprising the aforementioned distinct activities. In an especially preferred embodiment, the molecules of interest are polypeptides and a hetero-aggregate comprising these polypeptides is produced by co-expression of the at least two chimeric molecules in a host cell under conditions favorable to aggregation.

Advantageously, the above methods further include the step of isolating the higher order aggregate from the cell or from the growth medium of the cell.

In one embodiment, each chimeric protein comprising an SCE and a polypeptide of interest is produced in a separate and distinct host cell system and recovered (purified and isolated). The proteins are either recovered in soluble form or are solubilised. (Complete purification is desirable but not essential for subsequent aggregation.) Thereafter, a desired mixture of the two or more polypeptides is created and subjected to conditions that permit aggregation or polymerisation. Such conditions include physiological conditions or may involve the induction of aggregation, e.g., by “seeding” with a protein aggregate, by concentrating the mixture to increase molarity of the proteins, or by altering salinity, acidity, or other factors. The desired mixture may be 1:1 or may be at a ratio weighted in favor of one chimeric protein (e.g., weighted in favor of a polypeptide that has a lower association constant with its binding or interacting partner than another polypeptide whose collective activities are required to achieve a biological outcome). The different chimeric proteins co-polymerise with the seed and with each other because they comprise compatible SCE domains, and most preferably identical SCE domains.

In another embodiment, at least two distinct host cell systems are co-cultured, and the chimeric proteins are secreted into the common culture medium. The proteins can be co-purified from the medium or can be subjected to conditions favorable to aggregation to form higher order aggregates without prior purification.

In still another embodiment, the transgenes for two or more recombinant chimeric polypeptides are co-transfected into the same host cell, either on a single polynucleotide construct or multiple constructs. Such a host cell produces both recombinant polypeptides, which will form higher order aggregate in vivo under conditions favorable to aggregation. Alternatively, both recombinant polypeptides can be recovered in soluble form and subjected to conditions favorable to aggregation in vitro to form higher order aggregates.

The biological activity of the homo- or hetero-aggregates of the present invention can be assayed using standard techniques known to persons of skill in the art. For example, antigenic aggregates may be tested for immunogenicity by immunising an animal with the aggregates and assessing whether immune cells of the animal primed to attack such antigens are increased in number, activity, and ability to detect and destroy those antigens. Strength of immune response is measured by standard tests including: direct measurement of peripheral blood lymphocytes by means known to the art; natural killer cell cytotoxicity assays (see, e.g., Provinciali M. et al (1992, J. Immunol. Meth. 155: 19-24), cell proliferation assays (see, e.g., Vollenweider, I. and Groseurth, P. J. (1992, J. Immunol. Meth. 149: 133-135), immunoassays of immune cells and subsets (see, e.g., Loeffler, D. A., et al. (1992, Cytom. 13: 169-174); Rivoltini, L., et al. (1992, Can. Immunol. Immunother. 34: 241-251); or skin tests for cell-mediated immunity (see, e.g., Chang, A. E. et al (1993, Cancer Res. 53: 1043-1050). Alternatively, cytokine aggregates can be tested for their ability to confer the activity of the cytokine, e.g., the ability of SCE-GM-CSF to stimulate the proliferation of granulocytes and macrophages in vivo. Such techniques are well known to the skilled practitioner.

5. Applications

The present invention also provides practical applications of the higher order aggregates of the invention. Suitably, the invention contemplates the use of higher order homo-aggregates in a range of applications, including therapeutic, prophylactic and chemical process applications. In one embodiment of this type, the homo-aggregate comprises a therapeutic polypeptide for treating or preventing a particular disease or condition. For example, the therapeutic polypeptide may be a cytokine such as granulocyte/macrophage colony-stimulating factor (GM-CSF), which is a haematopoietic growth factor that stimulates the survival, proliferation, differentiation and function of myeloid cells and their precursors, particularly neutrophil and eosinophil granulocytes and monocytes/macrophages. GM-CSF is useful for treating a variety of haematopoietic conditions, including myelosuppressive disorders such as Acquired Immune Deficiency Syndrome (AIDS) and infectious diseases. It is also useful for treating cancers such as melanoma. Because higher order GM-CSF aggregates will have enhanced activity in accordance with the present invention (e;g., a higher potency and/or a prolonged circulating half-life), the frequency with which they must be used or administered is reduced, or the amount used or administered to achieve an effective dose is reduced. For example, a reduced quantity of aggregate would be necessary over the course of treatment than would otherwise be necessary if a non-aggregated form of GM-CSF were used alone for proliferation, differentiation and functional activation of hematopoietic progenitor cells, such as bone marrow cells. Other examples of therapeutically useful proteins which can be used to form homo-aggregates in accordance with the present invention are chemokine proteins, e.g., monocyte chemoattractant protein-1 (MCP-1), which may also be used inter alia for cancer treatment.

In an alternate embodiment, the homo-aggregate comprises a polypeptide having enzymatic activity, especially an activity considered to be of catalytic value in a chemical process. Higher order aggregates comprising such polypeptides can be used as a catalytic matrix for carrying out the chemical process.

Alternatively, the invention contemplates the use of higher order hetero-aggregates. In one embodiment of this type, the higher order hetero-aggregates comprise a plurality of antigens for modulating an immune response in an individual. Such multi-valent immunomodulating compositions may be administered alone or in combination with adjuvants that enhance the effectiveness of the compositions. In certain embodiments, the higher order aggregates will be particulate in nature and could be used advantageously to prime antigen presenting cells, especially dendritic cells, for high efficiency delivery of the antigens to both the MHC class I and/or MHC class II pathways of these cells. In this embodiment, the treated dendritic cells will elicit a strong immune response with very efficient generation of antigen-specific CTLs and T helper cells. Other antigen-presenting cells that could be primed with the aggregates of the invention include monocytes, macrophages, cells of myeloid lineage, B cells, dendritic cells or Langerhans cells. Methods for producing antigen-primed dendritic cells are described for example by Steinman et al. in U.S. Pat. No. 5,994,126.

In another embodiment, the higher order hetero-aggregates comprise a first chimeric polypeptide comprising interleukin-2 (IL-2) and a second chimeric polypeptide comprising Fas ligand. Such higher order aggregates could be useful in targeting certain leukemia or lymphoma cells, or recently activated T cells which bear both high affinity IL-2R and Fas.

In another embodiment, ordered aggregates are created comprising two or more enzymes, such as a first enzyme that catalyses one step of a chemical process and a second enzyme that catalyses a downstream step involving a “metabolic” product from the first enzymatic reaction. Such aggregates will generally increase the speed and/or efficiency of the chemical process due to the proximity of the first reaction products and the second catalyst enzyme.

From the foregoing, it will be apparent that the higher order aggregates can be used for the prevention or treatment of many conditions or deficiencies in patients by physicians and/or veterinarians. Accordingly, the invention contemplates in another aspect a pharmaceutical composition comprising a higher order aggregate of the invention, together with a pharmaceutically acceptable carrier and/or diluent. The amount of aggregates used in the treatment of various conditions will, of course, depend upon the severity of the condition being treated, the route of administration chosen, and the specific activity or purity of the higher order aggregate, and will be determined by the attending physician or veterinarian. Pharmaceutical compositions suitable for administration comprise the higher order aggregate in an effective amount and a pharmaceutically acceptable carrier.

Compositions of the present invention can be administered by a variety of routes, including, but not limited to, parenteral (e.g., injection, including but not limited to, intravenous, intraarterial, intramuscular, subcutaneous; inhalation, including but not limited to, intrabronchial, intranasal or oral inhalation, intranasal drops; topical) and non-parenteral (e.g., oral, including but not limited to, dietary; rectal).

The carriers will be non-toxic to recipients at the dosages and concentrations employed. The formulation used will vary according to the route of administration selected (e.g., solution, emulsion, capsule). For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers. See, generally, Remington's Pharmaceutical Science, 16th Edition, Mack, Ed. (1980). For inhalation, the compound can be solubilised and loaded into a suitable dispenser for administration (e.g., an atomiser, nebuliser or pressurised aerosol dispenser). Fusion proteins can be administered individually, together or in combination with other drugs or agents (e.g., other chemotherapeutic agents, immune system enhancers).

The present invention also contemplates immunopotentiating compositions comprising a higher order aggregate of the invention and optionally an adjuvant. Examples of adjuvants which may be effective include but are not limited to: aluminium hydroxide, N-acetyl-muramyl-L-threonyl-D-isoglutamine (thur-MDP), N-acetyl-nor-muramyl-L-alanyl-D-isoglutamine (CGP 11637, referred to as nor-MDP), N-acetylmuramyl-L-alany-D-isoglutaminyl-L-alaline-2-(1′-2′-dipalmitoyl-sn-glycero-3-hydroxyphosphoryloxy)-ethylamine (CGP 1983A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. For example, the effectiveness of an adjuvant may be determined by measuring the amount of antibodies resulting from the administration of the composition, wherein those antibodies are directed against one or more antigens presented by the treated cells of the composition In addition, if desired, the immunopotentiating composition may contain minor amounts of auxiliary substances such as wetting or emulsifying agents and/or pH buffering agents that enhance the effectiveness of the composition.

If desired, devices or compositions containing the immunopotentiating compositions suitable for sustained or intermittent release could be, in effect, implanted in the body or topically applied thereto for the relatively slow release of such materials into the body.

The immunopotentiating compositions are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch, magnesium carbonate, and the like. These compositions take the form of solutions, suspensions, tablets, pills, capsules, sustained release formulations or powders and contain 10%-95% of active ingredient, preferably 25%-70%.

Also encapsulated by the present invention is a method for treatment and/or prophylaxis of a disease or condition, comprising administering to a patient in need of such treatment an effective amount of a composition as broadly described above. The disease or condition may be caused by a pathogenic organism or a cancer as for example described above or it may be an autoimmune disease or allergy.

In some embodiments, the immunopotentiating composition of the invention is suitable for the treatment or prophylaxis of a cancer. Cancers which could be suitably treated in accordance with the practices of this invention include cancers of the lung, breast, ovary, cervix, colon, head and neck, pancreas, prostate, stomach, bladder, kidney, bone liver, oesophagus, brain, testicle, uterus, melanoma and the various leukemias and lymphomas.

In other embodiments, the immunopotentiating composition is suitable for treatment of, or prophylaxis against, a viral, bacterial or parasitic infection. Viral infections contemplated by the present invention include, but are not restricted to, infections caused by HIV, Hepatitis, Influenza, Japanese encephalitis virus, Epstein-Barr virus and respiratory syncytial virus. Bacterial infections include, but are not restricted to, those caused by Neisseria species, Meningococcal species, Haemophilus species Salmonella species, Streptococcal species, Legionella species and Mycohacterium species. Parasitic infections encompassed by the invention include, but are not restricted to, those caused by Plasmodium species, Schistosoma species, Leishmania species, Trypanosoma species, Toxoplasma species and Giardia species.

The above compositions or vaccines may be administered in a manner compatible with the dosage formulation, and in such amount as is therapeutically effective to alleviate patients from the disease or condition or as is prophylactically effective to prevent incidence of the disease or condition in the patient. The dose administered to a patient, in the context of the present invention, should be sufficient to effect a beneficial response in a patient over time such as a reduction or cessation of blood loss. The quantity of the composition or vaccine to be administered may depend on the subject to be treated inclusive of the age, sex, weight and general health condition thereof. In this regard, precise amounts of the composition or vaccine for administration will depend on the judgement of the practitioner. In determining the effective amount of the composition or vaccine to be administered in the treatment of a disease or condition, the physician may evaluate the progression of the disease or condition over time. In any event, those of skill in the art may readily determine suitable dosages of the composition or vaccine of the invention.

In order that the invention may be readily understood and put into practical effect, particular preferred embodiments will now be described by way of the following non-limiting examples.

### EXAMPLES

### Example 1

Portable OmpA Signal Peptide Construct

Molecules of interest including bioactive polypeptides can be assembled into higher order aggregates by covalent attachment of a portable construct comprising a signal peptide and a flexible linker to the amino-terminus or the carboxy-terminus of the individual bioactive polypeptides. In this example, the signal peptide linker comprises the sequence: MKKTAIAIAVALAGFATVAQAGGGGSGGGGSGGGGS*** [SEQ ID NO:133] or the sequence ***GSSGSGGGGSGGGGSTAIAIAVALAGFATVAQATKK [SEQ ID NO:134]. The first 21 amino acid residues of SEQ ID NO:133 and the last 21 amino acid residues of SEQ ID NO:134 are derived from the OmpA signal peptide. The remaining amino acid residues of these sequences represent shortened versions of a flexible hydrophilic linker that is routinely used, for example, in single-chain antibody production. Other flexible hydrophilic linkers have been reported and could be used in their place. The symbols *** symbolise the reactive group (e.g., a-halocarboxylic acid or ester such as iodoacetamide, an imide such as maleimide, a vinyl sulphone, or a disulphide) required for conjugation of the peptide linker to the bioactive polypeptides.

### Example 2

Assembly of Recombinant or Synthetic SCE-Chimeric Constructs

For illustration purposes, a recombinant or synthetic chimeric construct is assembled by linking together in the same reading frame a first nucleotide sequence encoding an SCE, a second nucleotide sequence encoding a peptide or polypeptide of interest and a third nucleotide sequence encoding a tag peptide, which facilitates purification of the construct. Optionally interposed between the first and second nucleotide sequences and the second and third nucleotide sequences are spacer-encoding oligonucleotides, which, when translated, space the polypeptide of interest from the SCE so that the SCE sequence does not interfere substantially with proper folding of the polypeptide of interest. The SCE may be linked to either the N-terminus or the C-terminus of a polypeptide of interest. The constructs encode fusion proteins, which are summarised by the following general formulae:

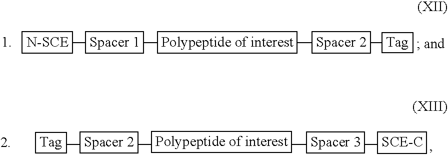

wherein:

the N-SCE is MKKTAIAIAVALAGFATVAQA [SEQ ID NO:136];

the SCE-C is TAIAIAVALAGFATVAQATKK [SEQ ID NO:138];

the polypeptide of interest is selected from murine or human GM-CSF [SEQ ID NO:140 and 142, respectively], murine or human IFN-β [SEQ ID NO:144 and 146, respectively], murine or human IL-1Ra [SEQ ID NO:148 and 150, respectively], murine or human IL-2 [SEQ ID NO:152 and 154, respectively], murine or human Fas ligand [SEQ ID NO:156 and 158, respectively], or HEL [SEQ ID NO:160], murine or human MCP-1 [SEQ ID NO:208 and 210, respectively];

the tag is selected from Flag (DYKDDDDK [SEQ ID NO:162]), His (HHHHHH [SEQ ID NO:164]) or Strep (AWRHPQFGG [SEQ ID NO:166]);

Spacer 1 is optional, and when present, is GS(GGGGS)ₙGSS [SEQ ID NO:167], wherein n=0-10;

Spacer 2 is optional, and when present, is GSS [SEQ ID NO:168]; and

Spacer 3 is optional, and when present, is GSSGS(GGGGS)ₙ [SEQ ID NO:169], wherein n=0-10.

For recombinant expression, nucleic acid constructs that encode the chimeric molecules of the invention are designed with appropriate translation initiation (e.g., ATG) and termination (e.g., TAA) signals if such signals are not already provided by the terminal elements of the constructs. These constructs can be inserted into appropriate expression vectors (e.g., a pET-28a(+) vector, which is commercially available from Novagen) for recombinant expression of the construct.

### Example 3

Self-Coalescing Murine GM-CSF Construct

A self-coalescing murine GM-CSF is producible using a suitable expression system that expresses the following nucleic acid sequence:

| | |
|---|---|
| [symbol] | [SEQ ID NO:185] |
| [symbol] GGATCCGGTGGTGGTGGATCCGGCTCGAGTTGGCTGCAGAATTTACTTTTCCTGGGCAT | |
| TGTGGTCTACAGCCTCTCAGCACCCACCCGCTCACCCATCACTGTCACCCGGCCTTGGAAGCATG | |
| TAGAGGCCATCAAAGAAGCCCTGAACCTCCTGGATGACATGCCTGTCACATTGAATGAAGAGGT | |
| AGAAGTCGTCTCTAACGAGTTCTCCTTCAAGAAGCTAACATGTGTGCAGACCCGCCTGAAGATA | |
| TTCGAGCAGGGTCTACGGGGCAATTTCACCAAACTCAAGGGCGCCTTGAACATGACAGCCAGCT | |
| ACTACCAGACATACTGCCCCCCAACTCCGGAAACGGACTGTGAAACACAAGTTACCACCTATGC | |
| GGATTTCATAGACAGCCTTAAAACCTTTCTGACTGATATCCCCTTTGAATGCAAAAAACCAGTCC | |
| AAAAAGGCTCGAGTGACTACAAGGACGATGACGACAAGTAATAA | |

wherein the boxed nucleotides encode N-SCE, the underlined nucleotides encode spacer 1, where n=1, the nucleotides in normal type face encode murine GM-CSF, the double underlined nucleotides encode Spacer 2, the italicised nucleotides encode the FLAG tag to facilitate purification and the nucleotides in bold type face are a tandem pair of translation termination codons.

Expression of the above construct, e.g., in E. coli, will produce a polypeptide with the following sequence:

| | |
|---|---|
| MKKTAIAIAVALAGFATVAQAGSGGGGSGSSWLQNLLFLGIVVYSLSAPTRSPITVTRPWK | [SEQ ID NO:186] |
| HVEAIKEALNLLDDMPVTLNEEVEVVSNEFSFKKLTCVQTRLKIFEQGLRGNFTKLKGALNMTASY | |
| YQTYCPPTPETDCETQVTTYADFIDSLKTFLTDIPFECKKPVQKGSSDYKDDDDK | |

### Example 4

Self-Coalescing Human GM-CSF Construct

A self-coalescing human GM-CSF is producible using a suitable expression system that expresses the following nucleic acid sequence:

| | |
|---|---|
| ATGGACTACAAGGACGATGACGACAAGGGCTCGAGTTGGCTGCAGAGCCTGCTGCTCTTG | [SEQ ID NO:187] |
| GGCACTGTGGCCTGCAGCATCTCTGCACCCGCCCGCTCGCCCAGCCGCAGCACGGAGCCCTGGG | |
| AGCATGTGAATGCCATCCAGGAGGCCCGGCGTCTCCTGAACCTGAGTAGAGACACTGCTGCTGA | |
| GATGAATGAAACAGTAGAAGTCATCTCAGAAATGTTTGACCTCCAGGAGCCGACCTGCCTACAG | |
| ACCCGCCTGGAGCTGTACAAGCAGGGCCTGCGGGGCAGCCTCACCAAGCTCAAGGGCCCCTTG | |
| ACCATGATGGCCAGCCACTACAAGCAGCACTGCCCTCCAACCCCGGAAACTTCCTGTGCAACCC | |
| AGATTATCACCTTTGAAAGTTTCAAAGAGAACCTGAAGGACTTTCTGCTTGTCATCCCCTTTGAC | |
| TGCTGGGAGCCAGTCCAGGAGGGCTCGAGTGGATCCGGTGGTGGTGGTAGCGGTGGTGGTGGA | |
| TCC [symbol] | |
| [symbol] | |

wherein the nucleotides in bold type face are a translation initiation codon, the italicised nucleotides encode the Flag tag to facilitate purification, the double underlined nucleotides encode Spacer 2, the nucleotides in normal type face encode human GM-CSF, the underlined nucleotides encode Spacer 3, where n=2 and the boxed nucleotides encode SCE-C.

Expression of the above construct, e.g., in E. coli, will produce a polypeptide with the following sequence:

| | |
|---|---|
| MDYKDDDDKGSSWLQSLLLLGTVACSISAPARSPSPSTQPWEHVNAIQEARRLLNLSRDTA | [SEQ ID NO:188] |
| AEMNETVEVISEMFDLQEPTCLQTRLELYKQGLRGSLTKLKGPLTMMASHYKQHCPPTPETSCATQI | |
| ITFESFKENLKDFLLVIPFDCWEPVQEGSSGSGGGGSGGGGSTAIAIAVALAGFATVAQATKK | |

### Example 5

Self-Coalescing Murine IFN-β Construct

A self-coalescing murine IFN-β is producible using a suitable expression system that expresses the following nucleic acid sequence:

| | |
|---|---|
| ATGCATCATCATCATCATCATGGCTCGAGTAACAACAGGTGGATCCTCCACGCTGCGTTC | [SEQ ID NO:189] |
| CTGCTGTGCTTCTCCACCACAGCCCTCTCCATCAACTATAAGCAGCTCCAGCTCCAAGAAAGGA | |
| CGAACATTCGGAAATGTCAGGAGCTCCTGGAGCAGCTGAATGGAAAGATCAACCTCACCTACA | |
| GGGCGGACTTCAAGATCCCTATGGAGATGACGGAGAAGATGCAGAAGAGTTACACTGCCTTTG | |
| CCATCCAAGAGATGCTCCAGAATGTCTTTCTTGTCTTCAGAAACAATTTCTCCAGCACTGGGTGG | |
| AATGAGACTATTGTTGTACGTCTCCTGGATGAACTCCACCAGCAGACAGTGTTTCTGAAGACAG | |
| TACTAGAGGAAAAGCAAGAGGAAAGATTGACGTGGGAGATGTCCTCAACTGCTCTCCACTTGA | |
| AGAGCTATTACTGGAGGGTGCAAAGGTACCTTAAACTCATGAAGTACAACAGCTACGCCTGGAT | |
| GGTGGTCCGAGCAGAGATCTTCAGGAACTTTCTCATCATTCGAAGACTTACCAGAAACTTCCAA | |
| AACGGCTCGAGTGGATCCGGTGGTGGTGGTAGCGGTGGTGGTGGTAGCGGTGGTGGTGGTAGC | |
| GGTGGTGGTGGTAGCGGTGGTGGTGGATCC [symbol] | |
| [symbol] | |

wherein the nucleotides in bold type face are a translation initiation codon, the italicised nucleotides encode the His tag to facilitate purification, the double underlined nucleotides encode Spacer 2, the nucleotides in normal type face encode murine IFN-β, the underlined nucleotides encode Spacer 3, where n=5 and the boxed nucleotides encode SCE-C.

Expression of the above construct, e.g., in E. coli, will produce a polypeptide with the following sequence:

| | |
|---|---|
| MHHHHHHGSSNNRWILHAAFLLCFSTTALSINYKQLQLQERTNIRKCQELLEQLNGKINLT | [SEQ ID NO:190] |
| YRADFKIPMEMTEKMQKSYTAFAIQEMLQNVFLVFRNNFSSTGWNETIVVRLLDELHQQTVFLKTV | |
| LEEKQEERLTWEMSSTALHLKSYYWRVQRYLKLMKYNSYAWMVVRAEIFRNFLIIRRLTRNFQNGS | |
| SGSGGGGSGGGGSGGGGSGGGGSGGGGSTAIAIAVALAGFATVAQATKK | |

### Example 6

Self-Coalescing Human IFN-β Construct

A self-coalescing human IFN-β is producible using a suitable expression system that expresses the following nucleic acid sequence:

| | |
|---|---|
| [symbol] | [SEQ ID NO:191] |
| [symbol] GGATCCGGTGGTGGTGGTAGCGGTGGTGGTGGATCCGGCTCGAGTACCAACAAGTGTCT | |
| CCTCCAAATTGCTCTCCTGTTGTGCTTCTCCACTACAGCTCTTTCCATGAGCTACAACTTGCTTGG | |
| ATTCCTACAAAGAAGCAGCAATTTTCAGTGTCAGAAGCTCCTGTGGCAATTGAATGGGAGGCTT | |
| GAATATTGCCTCAAGGACAGGATGAACTTTGACATCCCTGAGGAGATTAAGCAGCTGCAGCAGT | |
| TCCAGAAGGAGGACGCCGCATTGACCATCTATGAGATGCTCCAGAACATCTTTGCTATTTTCAG | |
| ACAAGATTCATCTAGCACTGGCTGGAATGAGACTATTGTTGAGAACCTCCTGGCTAATGTCTAT | |
| CATCAGATAAACCATCTGAAGACAGTCCTGGAAGAAAAACTGGAGAAAGAAGATTTTACCAGG | |
| GGAAAACTCATGAGCAGTCTGCACCTGAAAAGATATTATGGGAGGATTCTGCATTACCTGAAGG | |
| CCAAGGAGTACAGTCACTGTGCCTGGACCATAGTCAGAGTGGAAATCCTAAGGAACTTTTACTT | |
| CATTAACAGACTTACAGGTTACCTCCGAAACGGCTCGAGTGCTTGGCGTCACCCGCAGTTCGGTG | |
| GTTAATAA | |

wherein the boxed nucleotides encode N-SCE, the underlined nucleotides encode Spacer 1, where n=2, the nucleotides in normal type face encode human IFN-β, the double underlined nucleotides encode Spacer 2, the italicised nucleotides encode the Strep tag to facilitate purification and the nucleotides in bold type face are a tandem pair of translation termination codons.

Expression of the above construct, e.g., in E. coli, will produce a polypeptide with the following sequence:

| | |
|---|---|
| MKKTAIAIAVALAGFATVAQAGSGGGGSGGGGSGSSTNKCLLQIALLLCFSTFFALSMSYNL | [SEQ ID NO:192] |
| LGFLQRSSNFQCQKLLWQLNGRLEYCLKDRMNEDIPEEIKQLQQFQKEDAALTIYEMLQNIFAIFRQ | |
| DSSSTGWNETIVENLLANVYHQINHLKTVLEEKLEKEDFTRGKLMSSLHLKRYYGRILHYLKAKEYS | |
| HCAWTIVRVEILRNFYFINRLTGYLRNGSSAWRHPQFGG | |

### Example 7

Self-Coalescing Murine IL-1Ra Construct

A self-coalescing murine IL-1Ra is producible using a suitable expression system that expresses the following nucleic acid sequence:

| | |
|---|---|
| [symbol] | [SEQ ID NO:193] |
| [symbol] GGATCCGGTGGTGGTGGTAGCGGTGGTGGTGGTAGCGGTGGTGGTGGTAGCGGTGGTG | |
| GTGGTAGCGGTGGTGGTGGATCCGGCTCGAGTGAAATCTGCTGGGGACCCTACAGTCACCTAAT | |
| CTCTCTCCTTCTCATCCTTCTGTTTCATTCAGAGGCAGCCTGCCGCCCTTCTGGGAAAAGACCCT | |
| GCAAGATGCAAGCCTTCAGAATCTGGGATACTAACCAGAAGACCTTTTACCTGAGAAACAACCA | |
| GCTCATTGCTGGGTACTTACAAGGACCAAATATCAAACTAGAAGAAAAGATAGACATGGTGCCT | |
| ATTGACCTTCATAGTGTGTTCTTGGGCATCCACGGGGGCAAGCTGTGCCTGTCTTGTGCCAAGTC | |
| TGGAGATGATATCAAGCTCCAGCTGGAGGAAGTTAACATCACTGATCTGAGCAAGAACAAAGA | |
| AGAAGACAAGCGCTTTACCTTCATCCGCTCTGAGAAAGGCCCCACCACCAGCTTTGAGTCAGCT | |
| GCCTGTCCAGGATGGTTCCTCTGCACAACACTAGAGGCTGACCGTCCTGTGAGCCTCACCAACA | |
| CACCGGAAGAGCCCCTTATAGTCACGAAGTTCTACTTCCAGGAAGACCAAGGCTCGAGTGACTA | |
| CAAGGACGATGACGACAAGTAATAA | |

wherein the boxed nucleotides encode N-SCE, the underlined nucleotides encode Spacer 1, where n=5 , the nucleotides in normal type face encode murine IL-1Ra, the double underlined nucleotides encode Spacer 2, the italicised nucleotides encode the Flag tag to facilitate purification and the nucleotides in bold type face are a tandem pair of translation termination codons.

Expression of the above construct, e.g., in E. coli, will produce a polypeptide with the following sequence:

[SEQ ID NO:194]
MKKTAIAIAVALAGFATVAQAGSGGGGSGGGGSGGGGSGGGGSGGGGSGSSEICWGPYS
HLISLLLILLFHSEAACRPSGKRPCKMQAFRIWDTNQKTFYLRNNQLIAGYLQGPNIKLEEKIDMVPID
LHSVFLGIHGGKLCLSCAKSGDDIKLQLEEVNITDLSKNKEEDKRFTFIRSEKGPTTSFESAACPGWFL
CTTLEADRPVSLTNTPEEPLIVTKFYFQEDQGSSDYKDDDDK

### Example 8

Self-Coalescing Human IL-1Ra Construct

A self-coalescing human IL-1Ra is producible using a suitable expression system that expresses the following nucleic acid sequence:

| | |
|---|---|
| ATGCATCATCATCATCATCATGGCTCGAGTGAAATCTGCAGAGGCCTCCGCAGTCACCTA | [SEQ ID NO:195] |
| ATCACTCTCCTCCTCTTCCTGTTTCCATCAGAGACGATCTGCCGACCCTCTGGGAGAAAATCCAG | |
| CAAGATGCAAGCCTTCAGAATCTGGGATGTTAACCAGAAGACCTTCTATCTGAGGAACAACCAA | |
| CTAGTTGCTGGATACTTGCAAGGACCAAATGTCAATTTAGAAGAAAAGATAGATGTGGTACCCA | |
| TTGAGCCTCATGCTCTGTTCTTGGGAATCCATGGAGGGAAGATGTGCCTGTCCTGTGTCAAGTCT | |
| GGTGATGAGACCAGACTCCAGCTGGAGGCAGTTAACATCACTGACCTGAGCGAGAACAGAAAG | |
| CAGGACAAGCGCTTCGCCTTCATCCGCTCAGACAGCGGCCCCACCACCAGTTTTGAGTCTGCCG | |
| CCTGCCCCGGTTGGTTCCTCTGCACAGCGATGGAAGCTGACCAGCCCGTCAGCCTCACCAATAT | |
| GCCTGACGAAGGCGTCATGGTCACCAAATTCTACTTCCAGGAGGACGAGGGCTCGAGTGGATCC | |
| [symbol] | |
| [symbol] | |

wherein the nucleotides in bold type face are a translation initiation codon, the italicised nucleotides encode the His tag to facilitate purification, the double underlined nucleotides encode Spacer 2, the nucleotides in normal type face encode human IL1-Ra, the underlined nucleotides encode Spacer 3, where n=0 and the boxed nucleotides encode SCE-C.

Expression of the above construct, e.g., in E. coli, will produce a polypeptide with the following sequence:

[SEQ ID NO:196]
MHHHHHHGSSEICRGLRSHLITLLLFLFHSETICRPSGRKSSKMQAFRIWDVNQKTFYLRNN
QLVAGYLQGPNVNLEEKIDVVPIEPHALFLGIHGGKMCLSCVKSGDETRLQLEAVNITDLSENRKQD
KRFAFIRSDSGPTTSFESAACPGWFLCTAMEADQPVSLTNMPDEGVMVTKFYFQEDEGSSGSTAIAIA
VALAGFATVAQATKK

### Example 9

Self-Coalescing Murine IL-2 Construct

A self-coalescing murine IL-2 is producible using a suitable expression system that expresses the following nucleic acid sequence:

| | |
|---|---|
| ATGGCTTGGCGTCACCCGCAGTTCGGTGGTGGCTCGAGTTACAGCATGCAGCTCGCATC | [SEQ ID NO:197] |
| CTGTGTCACATTGACACTTGTGCTCCTTGTCAACAGCGCACCCACTTCAAGCTCCACTTCAAGCT | |
| CTACAGCGGAAGCACAGCAGCAGCAGCAGCAGCAGCAGCAGCAGCAGCAGCACCTGGAGCAG | |
| CTGTTGATGGACCTACAGGAGCTCCTGAGCAGGATGGAGAATTACAGGAACCTGAAACTCCCCA | |
| GGATGCTCACCTTCAAATTTTACTTGCCCAAGCAGGCCACAGAATTGAAAGATCTTCAGTGCCT | |
| AGAAGATGAACTTGGACCTCTGCGGCATGTTCTGGATTTGACTCAAAGCAAAAGCTTTCAATTG | |
| GAAGATGCTGAGAATTTCATCAGCAATATCAGAGTAACTGTTGTAAAACTAAAGGGCTCTGACA | |
| ACACATTTGAGTGCCAATTCGATGATGAGTCAGCAACTGTGGTGGACTTTCTGAGGAGATGGAT | |
| AGCCTTCTGTCAAAGCATCATCTCAACAAGCCCTCAAGGCTCGAGTGGATCCGGTGGTGGTGGA | |
| TCC [symbol] | |
| [symbol] | |

wherein the nucleotides in bold type face are a translation initiation codon, the italicised nucleotides encode the Strep tag to facilitate purification, the double underlined nucleotides encode Spacer 2, the nucleotides in normal type face encode murine IL-2, the underlined nucleotides encode Spacer 3, where n=1 and the boxed nucleotides encode SCE-C.

Expression of the above construct, e.g., in E. coli, will produce a polypeptide with the following sequence:

[SEQ ID NO:198]
MAWRHPQFGGGSSYSMQLASCVTLTLVLLVNSAPTSSSTSSSTAEAQQQQQQQQQQQQHL
EQLLMDLQELLSRMENYRNLKLPRMLTFKFYLPKQATELKDLQCLEDELGPLRHVLDLTQSKSFQL
EDAENFISNIRVTVVKLKGSDNTFECQFDDESATVVDFLRRWLAFCQSIISTSPQGSSGSGGGGSTALAI
AVALAGFATVAQATKK

### Example 10

Self-Coalescing Human IL-2 Construct

A self-coalescing human IL-2 is producible using a suitable expression system that expresses the following nucleic acid sequence:

| | |
|---|---|
| [symbol] | [SEQ ID NO:199] |
| [symbol] GGATCCGGCTCGAGTCCTACTTCAAGTTCTACAAAGAAAACACAGCTACAACTGGAGC | |
| ATTTACTGCTGGATTTACAGATGATTTTGAATGGAATTAATAATTACAAGAATCCCAAACTCACC | |
| AGGATGCTCACATTTAAGTTTTACATGCCCAAGAAGGCCACAGAACTGAAACATCTTCAGTGTC | |
| TAGAAGAAGAACTCAAACCTCTGAAGGAAGTGCTAAATTTAGCTCAAAGCAAAAACTTTCACTT | |
| AAGACCCAGGGACTTAATCAGCAATATCAACGTAATAGTTCTGGAACTAAAGGGATCTGAAAC | |
| AACATTCATGTGTGAATATGCTGATGAGACAGCAACCATTGTAGAATTTCTGAACAGATGGATT | |
| ACCTTTTCTCAAAGCATCATCTCAACACTGACTGGCTCGAGTGACTACAAGGACGATGACGACAAG | |
| TAATAA | |

wherein the boxed nucleotides encode N-SCE, the underlined nucleotides encode Spacer 1, where n=0, the nucleotides in normal type face encode human IL-2, the double underlined nucleotides encode Spacer 2, the italicised nucleotides encode the Flag tag to facilitate purification and the nucleotides in bold type face are a tandem pair of translation termination codons.

Expression of the above construct, e.g., in E. coli, will produce a polypeptide with the following sequence:

[SEQ ID NO:200]]
MKKTAIAIAVALAGFATVAQAGSGSSPTSSSTKKTQLQLEHLLLDLQMILNGINNYKNPKL
TRMLTFKFYMPKKATELKHLQCLEEELKPLKEVLNLAQSKNFHLRPRDLISNINVIVLELKGSETTFM
CEYADETATIVEFLNRWITFSQSIISTLTGSSDYKDDDDK

### Example 11

Self-Coalescing Murine Fas-L Construct

A self-coalescing murine Fas-ligand is producible using a suitable expression system that expresses the following nucleic acid sequence:

| | |
|---|---|
| [symbol] | [SEQ ID NO:201] |
| [symbol] GGATCCGGTGGTGGTGGATCCGGCTCGAGTCAGCAGCCCATGAATTACCCATGTCCCCA | |
| GATCTTCTGGGTAGACAGCAGTGCCACTTCATCTTGGGCTCCTCCAGGGTCAGTTTTTCCCTGTC | |
| CATCTTGTGGGCCTAGAGGGCCGGACCAAAGGAGACCGCCACCTCCACCACCACCTGTGTCACC | |
| ACTACCACCGCCATCACAACCACTCCCACTGCCGCCACTGACCCCTCTAAAGAAGAAGGACCAC | |
| AACACAAATCTGTGGCTACCGGTGGTATTTTTCATGGTTCTGGTGGCTCTGGTTGGAATGGGATT | |
| AGGAATGTATCAGCTCTTCCACCTGCAGAAGGAACTGGCAGAACTCCGTGAGTTCACCAACCAA | |
| AGCCTTAAAGTATCATCTTTTGAAAAGCAAATAGCCAACCCCAGTACACCCTCTGAAAAAAAAG | |
| AGCCGAGGAGTGTGGCCCATTTAACAGGGAACCCCCACTCAAGGTCCATCCCTCTGGAATGGGA | |
| AGACACATATGGAACCGCTCTGATCTCTGGAGTGAAGTATAAGAAAGGTGGCCTTGTGATCAAC | |
| GAAACTGGGTTGTACTTCGTGTATTCCAAAGTATACTTCCGGGGTCAGTCTTGCAACAACCAGC | |
| CCCTAAACCACAAGGTCTATATGAGGAACTCTAAGTATCCTGAGGATCTGGTGCTAATGGAGGA | |
| GAAGAGGTTGAACTACTGCACTACTGGACAGATATGGGCCCACAGCAGCTACCTGGGGGCAGT | |
| ATTCAATCTTACCAGTGCTGACCATTTATATGTCAAGATATCTCAACTCTCTCTGATCAATTTTGA | |
| GGAATCTAAGACCTTTTTCGGCTTGTATAAGCTTGGCTCGAGTCATCATCATCATCATCATTAATAA | |

wherein the boxed nucleotides encode N-SCE, the underlined nucleotides encode Spacer 1, where n=1, the nucleotides in normal type face encode murine Fas-L, the double underlined nucleotides encode Spacer 2, the italicised nucleotides encode the His tag to facilitate purification and the nucleotides in bold type face are a tandem pair of translation termination codons.

Expression of the above construct, e.g., in E. coli, will produce a polypeptide with the following 15 sequence:

| | |
|---|---|
| MKKTAIAIAVALAGFATVAQAGSGGGGSGSSQQPMNYPCPQIFWVDSSATSSWAPPGSVFP | [SEQ ID NO:202] |
| CPSCGPRGPDQRRPPPPPPPVSPLPPPSQPLPLPPLTPLKKKDHNTNLWLPVVFFMVLVALVGMGLG | |
| MYQLFHLQKELAELREFTNQSLKVSSFEKQIANPSTPSEKKEPRSVAHLTGNPHSRSWLEWEDTYGT | |
| ALISGVKYKKGGLVINETGLYFVYSKVYFRGQSCNNQPLNHKVYMRNSKYPEDLVLMEEKRLNYC | |
| TTGQIWAHSSYLGAVFNLTSADHLYVNISQLSLINFEESKTFFGLYKLGSSHHHHHH | |

### Example 12

Self-Coalescing Human Fas-L Construct

A self-coalescing human Fas-ligand is producible using a suitable expression system that expresses the following nucleic acid sequence:

| | |
|---|---|
| ATGGCTTGGCGTCACCCGCAGTTCGGTGGTGGCTCGAGTCAGCAGCCCTTCAATTACCC | [SEQ ID NO:203] |
| ATATCCCCAGATCTACTGGGTGGACAGCAGTGCCAGCTCTCCCTGGGCCCCTCCAGGCACAGTT | |
| CTTCCCTGTCCAACCTCTGTGCCCAGAAGGCCTGGTCAAAGGAGGCCACCACCACCACCGCCAC | |
| CGCCACCACTACCACCTCCGCCGCCGCCGCCACCACTGCCTCCACTACCGCTGCCACCCCTGAA | |
| GAAGAGAGGGAACCACAGCACAGGCCTGTGTCTCCTTGTGATGTTTTTCATGGTTCTGGTTGCCT | |
| TGGTAGGATTGGGCCTGGGGATGTTTCAGCTCTTCCACCTACAGAAGGAGCTGGCAGAACTCCG | |
| AGAGTCTACCAGCCAGATGCACACAGCATCATCTTTGGAGAAGCAAATAGGCCACCCCAGTCCA | |
| CCCCCTGAAAAAAAGGAGCTGAGGAAAGTGGCCCATTTAACAGGCAAGTCCAACTCAAGGTCC | |
| ATGCCTCTGGAATGGGAAGACACCTATGGAATTGTCCTGCTITCTGGAGTGAAGTATAAGAAGG | |
| GTGGCCTTGTGATCAATGAAACTGGGCTGTACTTTGTATATTCCAAAGTATACTTCCGGGGTCAA | |
| TCTTGCAACAACCTGCCCCTGAGCCACAAGGTCTACATGAGGAACTCTAAGTATCCCCAGGATC | |
| TGGTGATGATGGAGGGGAAGATGATGAGCTACTGCACTACTGGGCAGATGTGGGCCCGCAGCA | |
| GCTACCTGGGGGCAGTGTTCAATCTTACCAGTGCTGATCATTTATATGTCAACGTATCTGAGCTC | |
| TCTCTGGTCAATTTTGAGGAATCTCAGACGTTTTTCGGCTTATATAAGCTCGGCTCGAGTGGATC | |
| CGGTGGTGGTGGTAGCGGTGGTGGTGGATCC [symbol] | |
| [symbol] | |

wherein the nucleotides in bold type face are a translation initiation codon, the italicised nucleotides encode the Strep tag to facilitate purification, the double underlined nucleotides encode Spacer 2, the nucleotides in normal type face encode human Fas-L, the underlined nucleotides encode Spacer 3, where n=2 and the boxed nucleotides encode SCE-C.

Expression of the above construct, e.g., in E. coli, will produce a polypeptide with the following sequence:

[SEQ ID NO:204]
MAWRHPQFGGGSSQQPFNYPYPQIYWVDSSASSPWAPPGTVLPCPTSVPRRPGQRRPPPPP
PPPPLPPPPPPPPLPPLPLPPLKKRGNHSTGLCLLVMFFMVLVALVGLGLGMFQLFHLQKELAELREST
SQMHTASSLEKQIGHPSPPPEKKELRKVAHLTGKSNSRSMPLEWEDTYGIVLLSGVKYKKGGLVINE
TGLYFVYSKVYFRGQSCNNLPLSHKVYMRNSKYPQDLVMMEGKMMSYCTTGQMWARSSYLGAV
FNLTSADHLYVNVSELSLVNIFEESQTFFGLYKLGSSGSGGGGSGGGGSTAIAIAVALAGFATVAQAT
KK

### Example 13

Murine FasL with N-SCE and Murine IL-2 with SCE-C to Form Hetero-Aggregates

Self-coalescing murine Fas-ligand/IL-2 hetero-aggregates are produced by co-transfection of expression vectors, containing the nucleic acid constructs described in Examples 9 and 11, into cells (e.g., E. coli, CHO cells etc) and purification of the expressed polypeptide products over a Strepavidin-column and a Ni-chelate-column sequentially to ensure purification of hetero-aggregates only.

Both recombinant proteins will be produced in E. coli. After purification any already formed aggregates of N-SCE-murine Fas-L and murine IL-2-SCE-C will be broken up by Sonication/Tween 20 treatment and mixed together to allow co-aggregation.

### Example 14

Self-Coalescing HEL Construct

A self-coalescing HEL is producible using a suitable expression system that expresses the following nucleic acid sequence:

| | |
|---|---|
| ATGGACTACAAGGACGATGACGACAAGGGCTCGAGTAGGTCTTTGCTAATCTTGGTGCTT | [SEQ ID NO:205] |
| TGCTTCCTGCCCCTGGCTGCTCTGGGGAAAGTCTTTGGACGATGTGAGCTGGCAGCGGCTATGA | |
| AGCGTCACGGACTTGATAACTATCGGGGATACAGCCTGGGAAACTGGGTGTGTGTTGCAAAATT | |
| CGAGAGTAACTTCAACACCCAGGCTACAAACCGTAACACCGATGGGAGTACCGACTACGGAAT | |
| CCTACAGATCAACAGCCGCTGGTGGTGCAACGATGGCAGGACCCCAGGCTCCAGGAACCTGTG | |
| CAACATCCCGTGCTCAGCCCTGCTGAGCTCAGACATAACAGCGAGCGTGAACTGCGCGAAGAA | |
| GATCGTCAGCGATGGAAACGGCATGAGCGCGTGGGTCGCCTGGCGCAACCGCTGCAAGGGTAC | |
| CGACGTCCAGGCGTGGATCAGAGGCTGCCGGCTGGGCTCGAGTGGATCCGGTGGTGGTGGTAG | |
| CGGTGGTGGTGGTAGCGGTGGTGGTGGTAGCGGTGGTGGTGGTAGCGGTGGTGGTGGATCC\| [symbol] | |
| [symbol] | |
| [symbol] | |

wherein the nucleotides in bold type face are a translation termination codon, the italicised nucleotides encode the Flag tag to facilitate purification, the double underlined nucleotides encode Spacer 2, the nucleotides in normal type face encode HEL, the underlined nucleotides encode Spacer 3, where n=5 and the boxed nucleotides encode SCE-C.

Expression of the above construct, e.g., in E. coli, will produce a polypeptide with the following sequence:

| | |
|---|---|
| MDYKDDDDKGSSRSLLILVLCFLPLAALGKVFGRCELAAAMKRHGLDNYRGYSLGNWVC | [SEQ ID NO:206] |
| VAKFESNFNTQATNRNTDGSTDYGILQNSRWWCNDGRTPGSRNLCNIPCSALLSSDITASVNCAKKI | |
| VSDGNGMSAWVAWRNRCKGTDVQAWIRGCRLGSSGSGGGGSGGGGSGGGGSGGGGSGGGGSTAI | |
| AIAVALAGFATVAQATKK | |

### Example 15

Self-Coalescing Murine MCP-1 Construct

A self-coalescing murine MCP-1 is producible using a suitable expression system that expresses the following nucleic acid sequence:

| | |
|---|---|
| ATGAAAAAGACAGCTATCGCGATTGCAGTGGCACTGGCTGGTTTCGCTACCGTAGCGC | [SEQ ID NO:211] |
| AGGCCGGATCCGGCTCGAGTAAGATTTCCACACTTCTATGCCTCCTGCTCATAGCTACCACCATC | |
| AGTCCTCAGGTATTGGCTGGACCAGATGCGGTGAGCACCCCAGTCACGTGCTGTTATAATGTTG | |
| TTAAGCAGAAGATTCACGTCCGGAAGCTGAAGAGCTACAGGAGAATCACAAGCAGCCAGTGTC | |
| CCCGGGAAGCTGTGATCTTCAGGACCATACTGGATAAGGAGATCTGTGCTGACCCCAAGGAGA | |
| AGTGGGTTAAGAATTCCATAAACCACTTGGATAAGACGTCTCGAACGGGCTCGAGTGCTTGGCG | |
| TCACCCGCAGTTCGGTGGTTAATAA | |

wherein the boxed nucleotides encode N-SCE, the underlined nucleotides encode Spacer 1, where n=0, the nucleotides in normal type face encode murine MCP-1, the double underlined nucleotides encode Spacer 2, the italicised nucleotides encode the Strep tag to facilitate purification and the nucleotides in bold type face are tandem pair of translation termination codons.

Expression of the above construct, e.g., in E. coli, will produce a polypeptide with the following sequence:

[SEQ ID NO:212]
MKKTAIAIAVALAGFATVAQAGSGSSKISTLLCLLLIATTISPQVLAGPDAVSTPVTCCYNV
VKQKIHVRKLKSYRRITSSQCPREAVIFRTILDKEICADPKEKWVKNSINHLDKTSRTGSSAWRHPQF
GG

### Example 16

Self-Coalescing Human MCP-1 Construct

A self-coalescing human MCP-1 is producible using a suitable expression system that expresses the following nucleic acid sequence:

| | |
|---|---|
| [symbol] | [SEQ ID NO:213] |
| [symbol] GGATCCGGCTCGAGTAAAGTCTCTGCCGCCCTTCTGTGCCTGCTGCTCATAGCAGCCAC | |
| CTTCATTCCCCAAGGGCTCGCTCAGCCAGATGCAATCAATGCCCCAGTCACCTGCTGTTATAACT | |
| TCACCAATAGGAAGATCTCAGTGCAGAGGCTCGCGAGCTATAGAAGAATCACCAGCAGCAAGT | |
| GTCCCAAAGAAGCTGTGATCTTCAAGACCATTGTGGCCAAGGAGATCTGTGCTGACCCCAAGCA | |
| GAAGTGGGTTCAGGATTCCATGGACCACCTGGACAAGCAAACCCAAACTCCGAAGACTGGCTC | |
| GAGTCATCATCATCATCATCATTAATAA | |

wherein the boxed nucleotides encode N-SCE, the underlined nucleotides encode Spacer 1, where n=0, the nucleotides in normal type face encode human MCP-1, the double underlined nucleotides encode Spacer 2, the italicised nucleotides encode the His tag to facilitate purification and the nucleotides in bold type face are translation termination codons.

Expression of the above construct, e.g., in E. coli, will produce a polypeptide with the following sequence:

| | |
|---|---|
| MKKTAIAIAVALAGFATVAQAGSGSSKVSAALLCLLLIAATFIPQGLAQPDAINAPVTCCYN | [SEQ ID NO:214] |
| FTNRKISVQRLASYRRITSSKCPKEAVIFKTIVAKEICADPKQKWVQDSMDHLDKQTQTPKTGSSHH | |
| HHHH | |

### Example 17

Chemically Synthesised Chimeric Peptide Constructs

Chemically synthesised chimeric peptide constructs can be constructed according to the following general formulae

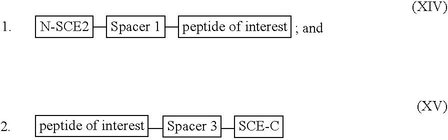

wherein:

N-SCE2 is KKTAIAIAVALAGFATVAQA [SEQ ID NO:215];

SCE-C is as defined in Example 2;

Spacers 1 and 3 are as defined in Example 2; and

the peptide of interest is selectable, for example, from metabolic peptides, cytokine peptides, peptides from cytokine receptors, effector peptides and antigenic peptides.

### Example 18

Synthetic Self-Coalescing Human a ACTH Chimeric Peptide

A self-coalescing human ACTH peptide is chemically synthesised with the following amino acid sequence:

| | |
|---|---|
| [symbol] GSGGGGSGSSSYSMEHFRWGKPVGKKRRPVKVYPNGAED | [SEQ ID NO:216] |
| ESAEAFPLEF | |

wherein the boxed residues are N-SCE2, the underlined residues are spacer 1, where n=1 and the residues in normal type face are a human ACTH peptide.

### Example 19

Synthetic Self-Coalescing Murine ACTH Chimeric Peptide

A self-coalescing murine ACTH peptide is chemically synthesised with the following amino acid sequence:

| | |
|---|---|
| SYSMEHFRWGKPVGKKRRPVKVYPNVAENESAEAFPLEFGSSGS [symbol] | [SEQ ID NO:217] |
| [symbol] | |

wherein the residues in normal type face are a murine ACTH peptide, the underlined residues are spacer 1, where n=0 and the boxed residues are SCE-C.

### Example 20

Synthetic Self-Coalescing α-MSH Chimeric Peptide

A self-coalescing α-MSH peptide is chemically synthesised with the following amino acid sequence:

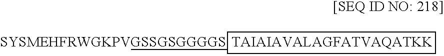

wherein the residues in normal type face are a murine ACTH peptide, the underlined residues are spacer 1, where n=1 and the boxed residues are SCE-C.

### Example 21

Synthetic Self-Coalescing Human β-MSH Chimeric Peptide

A self-coalescing β-MSH peptide is chemically synthesised with the following amino acid sequence:

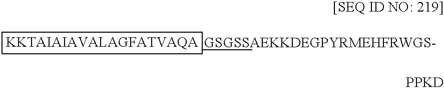

wherein the boxed residues are N-SCE2, the underlined residues are spacer 1, where n=0 and the residues in normal type face are a human β-MSH peptide.

### Example 22

Synthetic Self-Coalescing Murine β-MSH Chimeric Peptide

A self-coalescing β-MSH peptide is chemically synthesised with the following amino acid sequence:

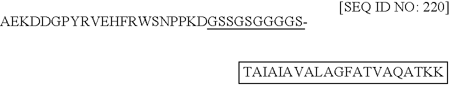

wherein the residues in normal type face are a murine β-MSH peptide, the underlined residues are spacer 1, where n=1 and the boxed residues are SCE-C.

### Example 23

Synthetic Self-Coalescing γ-MSH Chimeric Peptide

A self-coalescing γ-MSH peptide is chemically synthesised with the following amino acid sequence:

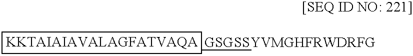

wherein the boxed residues are N-SCE2, the underlined residues are spacer 1, where n=0 and the residues in normal type face are a human γ-MSH peptide.

### Example 24

Synthetic Self-Coalescing Angiotensin I Chimeric Peptide

A self-coalescing angiotensin I peptide is chemically synthesised with the following amino acid sequence:

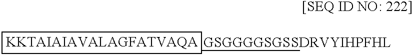

wherein the boxed residues are N-SCE2, the underlined residues are spacer 1, where n=1 and the residues in normal type face are an angiotensin I peptide.

### Example 25

Synthetic Self-Coalescing Angiotensin II Chimeric Peptide

A self-coalescing angiotensin II peptide is chemically synthesised with the following amino acid sequence:

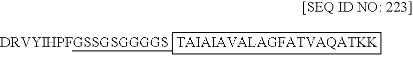

wherein the residues in normal type face are an angiotensin III peptide, the underlined residues are spacer 1, where n=1 and the boxed residues are SCE-C.

### Example 25

Synthetic Self-Coalescing Angiotensin III Chimeric Peptide

A self-coalescing angiotensin III peptide is chemically synthesised with the following amino acid sequence:

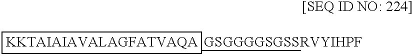

wherein the boxed residues are N-SCE2, the underlined residues are spacer 1, where n=1 and the residues in normal type face are an angiotensin III peptide.

### Example 26

Synthetic Self-Coalescing Human GHRH Chimeric Peptide I

A self-coalescing human growth hormone releasing hormone (GHRH) peptide is chemically synthesised with the following amino acid sequence:

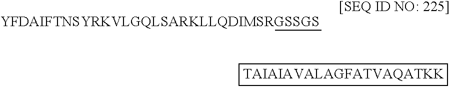

wherein the residues in normal type face are a human GHRH peptide, the underlined residues are spacer 1, where n=0 and the boxed residues are SCE-C, and wherein the Tyr at position 1 of the GHRH peptide is acetylated, the Phe at position 2 is in the D-isomeric form and the Arg at position 20 is amidated.

### Example 27

Synthetic Self-Coalescing Human GHRH Chimeric Peptide II

A self-coalescing human growth hormone releasing hormone (GHRH) peptide is chemically synthesised with the following amino acid sequence:

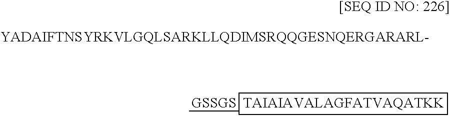

wherein the residues in normal type face are a human GHRH peptide, the underlined residues are spacer 1, where n=0 and the boxed residues are SCE-C.

### Example 28

Synthetic Self-Coalescing Murine GHRH Chimeric Peptide

A self-coalescing murine growth hormone releasing hormone (GHRH) peptide is chemically synthesised with the following amino acid sequence:

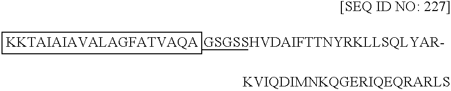

wherein the boxed residues are N-SCE2, the underlined residues are spacer 1, where n=0 and the residues in normal type face are a murine GHRH peptide.

### Example 29

Synthetic Self-Coalescing Human IL-1β Chimeric Peptide I

A self-coalescing human IL-1β peptide is chemically synthesised with the following amino acid sequence:

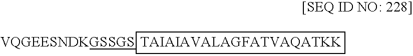

wherein the residues in normal type face are a human IL-1β (aa 163-171) peptide, the underlined residues are spacer 1, where n=0 and the boxed residues are SCE-C.

### Example 30

Synthetic Self-Coalescing Human IL-1β Chimeric Peptide II

A self-coalescing human IL-1βpeptide is chemically synthesised with the following amino acid sequence:

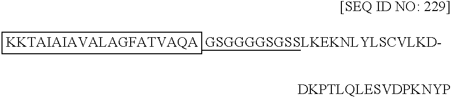

wherein the boxed residues are N-SCE2, the underlined residues are spacer 1, where n=1 and the residues in normal type face are a human IL-1β (aa 178-207) peptide.

### Example 31

Synthetic Self-Coalescing Human IL-2 Chimeric Peptide I

A self-coalescing human IL-2 peptide is chemically synthesised with the following amino acid sequence:

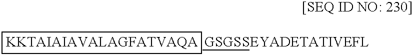

wherein the boxed residues are N-SCE2, the underlined residues are spacer 1, where n=0 and the residues in normal type face are a peptide from human IL-2 (126-138).

### Example 32

Synthetic Self-Coalescing Human IL-2 Chimeric Peptide II

A self-coalescing human IL-2 peptide is chemically synthesised with the following amino acid sequence:

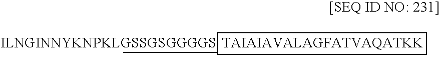

wherein the residues in normal type face are a peptide from human IL-2 (44-56), the underlined residues are spacer 1, where n=1 and the boxed residues are SCE-C.

### Example 33

Synthetic Self-Coalescing Human IL-2 Chimeric Peptide III

A self-coalescing human IL-2 peptide is chemically synthesised with the following amino acid sequence:

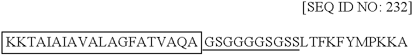

wherein the boxed residues are N-SCE2, the underlined residues are spacer 1, where n=1 and the residues in normal type face are a peptide from human IL-2 (60-70).

### Example 34

Synthetic Self-Coalescing Human TNF-α Chimeric Peptide I

A self-coalescing human TNF-α peptide is chemically synthesised with the following amino acid sequence:

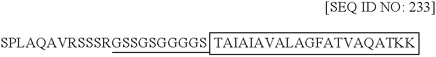

wherein the residues in normal type face are a peptide from human TNF-α (aa 71-82), the underlined residues are spacer 1, where n=1 and the boxed residues are SCE-C.

### Example 35

Synthetic Self-Coalescing Human TNF-α Chimeric Peptide II

A self-coalescing human TNF-α peptide is chemically synthesised with the following amino acid sequence:

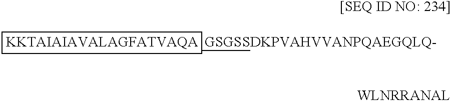

wherein the boxed residues are N-SCE2, the underlined residues are spacer 1, where n=0 and the residues in normal type face are a peptide from TNF-α (10-36).

### Example 36

Synthetic Self-Coalescing Human TNF-α Chimeric Peptide III

A self-coalescing human TNF-α peptide is chemically synthesised with the following amino acid sequence:

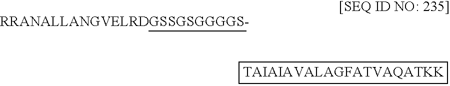

wherein the residues in normal type face are a peptide from TNF-α (31-45), the underlined residues are spacer 1, where n=1 and the boxed residues are SCE-C.

### Example 37

Synthetic Self-Coalescing Human Cys-BAFF-R Chimeric Peptide I

A self-coalescing human Cys-BAFF receptor peptide is chemically synthesised with the following amino acid sequence:

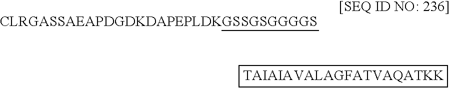

wherein the residues in normal type face are a peptide from human Cys-BAFF-R (aa 108-129), the underlined residues are spacer 1, where n=1 and the boxed residues are SCE-C.

### Example 38

Synthetic Self-Coalescing Human Cys-BAFF-R Chimeric Peptide II

A self-coalescing human Cys-BAFF receptor peptide is chemically synthesised with the following amino acid sequence:

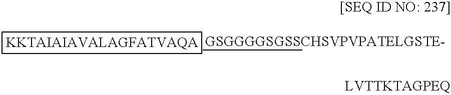

wherein the boxed residues are N-SCE2, the underlined residues are spacer 1, where n=1 and the residues in normal type face are a peptide from human Cys-BAFF-R (aa 159-183).

### Example 39

Synthetic Self-Coalescing Human P55-TNF-R Chimeric Peptide

A self-coalescing human P55-TNF receptor peptide is chemically synthesised with the following amino acid sequence:

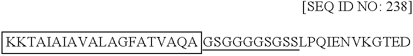

wherein the boxed residues are N-SCE2, the underlined residues are spacer 1, where n=1 and the residues in normal type face are a peptide from human P55-TNF-R.

### Example 40

Synthetic Self-Coalescing Human P75-TNF-R Chimeric Peptide

A self-coalescing human P75-TNF receptor peptide is chemically synthesised with the following amino acid sequence:

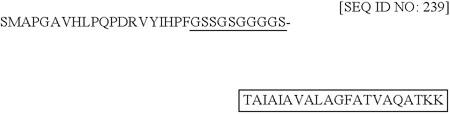

wherein the residues in normal type face are a peptide from human P75-TNF-R, the underlined residues are spacer 1, where n=1 and the boxed residues are SCE-C.

### Example 41

Synthetic Self-Coalescing IL-6-R Chimeric Peptide

A self-coalescing human IL-6 receptor peptide is chemically synthesised with the following amino acid sequence:

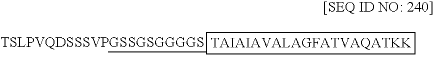

wherein the residues in normal type face are a peptide from human IL-6-R, the underlined residues are spacer 1, where n=1 and the boxed residues are SCE-C.

### Example 42

Synthetic Self-Coalescing L-Selectin Chimeric Peptide

A self-coalescing human L-selectin peptide is chemically synthesised with the following amino acid sequence:

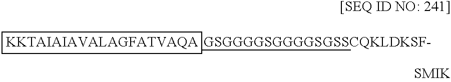

wherein the boxed residues are N-SCE2, the underlined residues are spacer 1, where n=2 and the residues in normal type face are a peptide from human L-selectin.

### Example 43

Synthetic Self-Coalescing MUC-1 Chimeric Peptide

A self-coalescing human MUC-1 (Mucin-1) peptide, which is useful for the preparation of tumor antigen vaccines, is chemically synthesised with the following amino acid sequence:

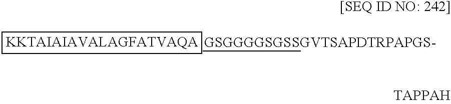

wherein the boxed residues are N-SCE2, the underlined residues are spacer 1, where n=1 and the residues in normal type face are a human MUC-1 peptide.

### Example 44

Synthetic Self-Coalescing Ovalbumin Chimeric Peptide I

A self-coalescing ovalbumin (OVA) peptide, which is useful for the preparation of immunopotentiating compositions, is chemically synthesised with the following amino acid sequence:

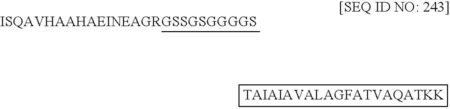

wherein the residues in normal type face are a peptide from OVA (aa 323-339), the underlined residues are spacer 1, where n=1 and the boxed residues are SCE-C.

### Example 45

Synthetic Self-Coalescing Ovalbumin Chimeric Peptide II

A self-coalescing ovalbumin (OVA) peptide, which is useful for the preparation of immunopotentiating compositions, is chemically synthesised with the following amino acid sequence:

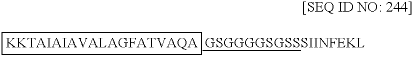

wherein the boxed residues are N-SCE2, the underlined residues are spacer 1, where n=1 and the residues in normal type face are a peptide from OVA (aa 257-264).

### Example 46

Synthetic Self-Coalescing HIV gp120 Chimeric Peptide I

A self-coalescing HIV gp120 peptide, which is useful for the preparation of immunopotentiating compositions, is chemically synthesised with the following amino acid sequence:

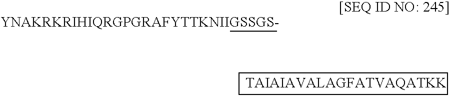

wherein the residues in normal type face are a peptide from HIV gp120, the underlined residues are spacer 1, where n=0 and the boxed residues are SCE-C.

### Example 47

Synthetic Self-Coalescing HIV gp120 Chimeric Peptide II

A self-coalescing HIV gp120 peptide, which is useful for the preparation of immunopotentiating compositions, is chemically synthesised with the following amino acid sequence:

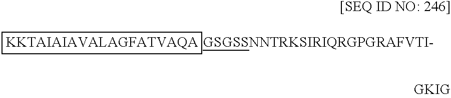

wherein the boxed residues are N-SCE2, the underlined residues are spacer 1, where n=0 and the residues in normal type face are a peptide from HIV gp120 (aa 307-331).

### Example 48

Synthetic Self-Coalescing HIV gp120 Chimeric Peptide III

A self-coalescing HIV gp120 peptide, which is useful for the preparation of immunopotentiating compositions, is chemically synthesised with the following amino acid sequence:

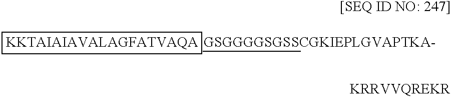

wherein the boxed residues are N-SCE2, the underlined residues are spacer 1, where n=1 and the residues in normal type face are a peptide from HIV gp120.

### Example 49

Synthetic Self-Coalescing HIV gp41 Chimeric Peptide

A self-coalescing HIV gp41 peptide, which is useful for the preparation of immunopotentiating compositions, is chemically synthesised with the following amino acid sequence:

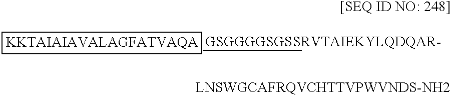

wherein the boxed residues are N-SCE2, the underlined residues are spacer 1, where n=1 and the residues in normal type face are a peptide from HIV gp41.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. An isolated or purified higher order aggregate comprising a plurality of chimeric molecules, wherein each chimeric molecule comprises at least one self-coalescing element, which is obtainable or derivable from a membrane translocating sequence or variant thereof, and which is fused, linked or otherwise associated with a molecule of interest, and wherein the or each self-coalescing element is capable of causing an individual chimeric molecule to coalesce with other chimeric molecules into higher order aggregates under conditions favorable to aggregation, wherein at least one chimeric molecule of the aggregate is other than a chimeric molecule selected from the group consisting of: a B cell activating fusion protein comprising a B cell surface immunoglobulin binding domain and a signal peptide, wherein a catalytic product of the precursor is capable of inducing B cell mitogenesis; and a fusion protein comprising protein L and ompA.

2. The aggregate of claim 1, wherein the self-coalescing element is from about 8 to about 35 amino acid residues in length.

3. The aggregate of claim 1, wherein the amino acid sequence of the self-coalescing element has from about 60% to about 95% small or hydrophobic amino acid residues or modified forms thereof.

4. The aggregate of claim 1, wherein the self-coalescing element is a membrane translocation sequence.

5. The aggregate of claim 4, wherein the membrane translocation sequence is a naturally occurring signal sequence or variant thereof, which has the ability to aggregate into higher order aggregates under physiological conditions.

6. The aggregate of claim 4, wherein the membrane translocation sequence is obtainable from an organism selected from the group consisting of bacteria, mycobacteria, viruses, protozoa, yeast, plants and animals.

7. The aggregate of claim 4, wherein the membrane translocation sequence is obtainable from an animal selected from the group consisting of insects, avians, reptiles, fish and mammals.

8. The aggregate of claim 4, wherein the membrane translocation sequence is obtainable from bacteria.

9. The aggregate of claim 8, wherein the membrane translocation sequence comprises an amino acid sequence selected from the group consisting of SEQ ID NO:12-90 and biologically active fragments thereof.

10. The aggregate of claim 8, wherein the membrane translocation sequence comprises an amino acid sequence selected from the group consisting of SEQ ID NO:67, 68, 69, 70, 71, 72, 73, 74, 75, 76, 77, 78, 79, 80, 81, 83, 84, 85 and 87 and biologically active fragments thereof.

11. The aggregate of claim 8, wherein the membrane translocation sequence comprises an amino acid sequence that is encoded by a nucleic acid sequence that hybridises under at least low stringency conditions to a sequence selected from the group consisting of SEQ ID NO:91-132.

12. The aggregate of claim 8, wherein the membrane translocation sequence comprises an amino acid sequence that is encoded by a nucleic acid sequence that hybridises under at least low stringency conditions to a sequence selected from the group consisting of SEQ ID NO:126-132.

13. The aggregate of claim 1, wherein the molecule of interest is an organic compound selected from the group consisting of drugs, metabolites, pesticides and herbicides.

14. The aggregate of claim 1, wherein the molecule of interest is an organic polymer.

15. The aggregate of claim 14, wherein the organic polymer is selected from the group consisting of polypeptides and polynucleotides.

16. The aggregate of claim 1, wherein the molecule of interest is a polypeptide selected from the group consisting of enzymes, receptors, antigen-binding molecules, ligand-binding polypeptides, metal-binding polypeptides, light-harvesting polypeptides, light spectrum-modifying polypeptides, regulatory polypeptides, chemokines, cytokines, interleukins, growth factors, interferons, metabolic polypeptides, immunopotentiating polypeptides, iummunosuppressing polypeptides, angiogenic polypeptides, anti-angiogenic polypeptides, antigenic polypeptides, and their biologically active fragments.

17. The aggregate of claim 1, wherein the molecule of interest is a polypeptide selected from the group consisting of cytokines, growth factors and hormones.

18. The aggregate of claim 17, wherein the polypeptide is selected from the group consisting of interferon-α, interferon-β, interferon-γ, interleukin-1, interleukin-2, interleukin-3, interleukin4, interleukin-5, interleukin-6, interleukin-7, interleukin-8, interleukin-9, interleukin-10, interleukin-11, interleukin-12, interleukin-13, interleukin-14, interleukin-15, interleukin-16, erythropoietin, colony-stimulating factor-1, granulocyte colony-stimulating factor, granulocyte-macrophage colony-stimulating factor, leukemia inhibitory factor, tumor necrosis factor, lymphotoxin, platelet-derived growth factor, fibroblast growth factors, vascular endothelial cell growth factor, epidermal growth factor, transforming growth factor-β, transforming growth factor-α, thrombopoietin, stem cell factor, oncostatin M, amphiregulin, Mullerian-inhibiting substance, B-cell growth factor, macrophage migration inhibiting factor, monocyte chemoattractant protein, endostatin, and angiostatin and their biologically active fragments.

19. The aggregate of claim 1, wherein the molecule of interest is a polypeptide antigen.

20. The aggregate of claim 19, wherein the polypeptide antigen is selected from the group consisting of viral antigens, bacterial antigens, protozoan antigens, microbial antigens, tumor antigens, self-antigens and auto-antigens.

21. The aggregate of claim 19, wherein the polypeptide antigen is derived from a virus selected from the group consisting of human immunodeficiency viruses (HIV), papilloma viruses, polioviruses, influenza viruses, Rous sarcoma viruses, encephalitis-causing viruses, herpes viruses and hepatitis viruses.

22. The aggregate of claim 19, wherein the polypeptide antigen is derived from a bacterium selected from the group consisting of Neisseria species, Meningococcal species, Haemophilus species, Salmonella species, Streptococcal species, Legionella species and Mycobacterium species.

23. The aggregate of claim 19, wherein the polypeptide antigen is derived from a protozoan selected from the group consisting of Plasmodium species, Schistosoma species, Leishmania species, Trypanosoma species, Toxoplasma species and Giardia species.

24. The aggregate of claim 19, wherein the polypeptide antigen is derived from a cancer or tumor selected from the group consisting of melanoma, lung cancer, breast cancer, cervical cancer, prostate cancer, colon cancer, pancreatic cancer, stomach cancer, bladder cancer, kidney cancer, post transplant lymphoproliferative disease (PTLD) and Hodgkin's Lymphoma.

25. The aggregate of claim 1, wherein the molecule of interest is a metabolic polypeptide selected from the group consisting of compound-absorbing polypeptides, compound-binding polypeptides, compound-uptaking polypeptides, compound-excreting polypeptides, compound-distributing polypeptides, compound-transporting polypeptides, compound-processing polypeptides, compound-converting polypeptides and compound-degrading polypeptides.

26. The aggregate of claim 25, wherein the metabolic polypeptide is selected from the group consisting of drug-metabolising polypeptides, drug-binding polypeptides, ornithine transcarbamylase, arginosuccinate synthetase, glutamine synthetase, glycogen synthetase, glucose-6-phosphatase, succinate dehydrogenase, glucokinase, insulin, pyruvate kinase, acetyl CoA carboxylase, fatty acid synthetase, alanine aminotransferase, glutamate dehydrogenase, ferritin, low density lipoprotein (LDL) receptor, P450 enzymes and alcohol dehydrogenase.

27. The aggregate of claim 1, wherein the molecule of interest is a peptide selected from the group consisting of T cell epitopes, B cell epitopes, cytokine peptides, chemokine peptides, neuropeptides, anti-inflammatory peptides and receptor ligand peptides.

28. The aggregate of claim 1, wherein the molecule of interest is a hormone.

29. The aggregate of claim 28, wherein the hormone is selected from the group consisting of growth hormones, sex hormones, thyroid hormones, pituitary hormones and melanocyte stimulating hormones.

30. The aggregate of claim 28, wherein the hormone is selected from the group consisting of estrogens, anti-estrogens, progestins, antiprogestin, androgens and anti-androgens.

31. The aggregate of claim 28, wherein the hormone is a thyroid hormone selected from the group consisting of triiodothyronne, thyroxine, propylthiouracil, methimazole, and iodixode.

32. The aggregate of claim 28, wherein the hormone is a gastrointestinal hormones selected from the group consisting of gastrin, glucagon, secretin, cholecystokinin, gastric inhibitory peptide, vasoactive intestinal peptide, substance P, glucagon-like immunoreactivity peptide, somatostatin, bombesin and neurotensin.

33. The aggregate of claim 28, wherein the hormone is a pituitary hormone selected from the group consisting of corticotropin, sumutotropin, oxytocin, and vasopressin.

34. The aggregate of claim 28, wherein the hormone is an adrenal cortex hormone selected from the group consisting of adrenocorticotropic hormone, aldosterone, cortisol, corticosterone, deoxycorticosterone and dehydroepiandrosterone.

35. The aggregate of claim 28, wherein the hormone is selected from the group consisting of prednisone, betamethasone, vetamethasone, cortisone, dexamethasone, flunisolide, hydrocortisone, methylprednisolone, paramethasone acetate, prednisolone and triamcinolone fludrocortisone.

36. The aggregate of claim 1, comprising identical, or substantially similar, molecules of interest.

37. The aggregate of claim 1, comprising different molecules of interest.

38. The aggregate of claim 1, wherein the chimeric molecule is formed by chemical synthesis.

39. The aggregate of claim 1, wherein a self-coalescing element is covalently attached to a molecule of interest by chemical crosslinking.

40. The aggregate of claim 39, wherein the self-coalescing element is chemical crosslinked to the molecule of interest using a chemical crosslinking agent.

41. The aggregate of claim 39, wherein the self-coalescing element is chemical crosslinked to the molecule of interest using a homobifunctional crosslinking agent.

42. The aggregate of claim 39, wherein the self-coalescing element is chemical crosslinked to the molecule of interest using a heterobifunctional crosslinking agent.

43. The aggregate of claim 1, wherein the chimeric molecule is formed by recombinant means.

44. The aggregate of claim 1, wherein the self-coalescing element is attached to the molecule of interest such that, on self-assembly of the chimeric molecule into the higher order aggregate, the molecule of interest is exposed to the exterior of the aggregate.

45. The aggregate of claim 1, wherein the self-coalescing element is spaced from the molecule of interest by a linker or spacer molecule.

46. The aggregate of claim 45, wherein the linker or spacer molecule spaces the molecule of interest from the self-coalescing element sufficiently so as to promote the proper folding of the molecule of interest.

47. The aggregate of claim 45, wherein the linker or spacer molecule spaces the molecule of interest from the self-coalescing element sufficiently such that the molecule of interest retains a desired activity when the chimeric molecule forms aggregates with other chimeric molecules.

48. The aggregate of claim 45, wherein the linker or spacer molecule is from about 1 to about 100 atoms in length.

49. The aggregate of claim 45, wherein the linker or spacer molecule is from about 1 to about 50 amino acid residues in length.

50. The aggregate of claim 45, wherein the linker or spacer molecule is an amino acid sequence selected from the group consisting of SEQ ID NO:167, 169, 171, 173, 175, 179, 181 and 183.

51. An isolated or purified higher order aggregate comprising a plurality of chimeric molecules, wherein each chimeric molecule comprises at least one self-coalescing element, which is obtainable or derivable from a membrane translocating sequence or variant thereof, and which is fused, linked or otherwise associated with a molecule of interest, and wherein the or each self-coalescing element is capable of causing an individual chimeric molecule to coalesce with other chimeric molecules into higher order aggregates under conditions favorable to aggregation, wherein at least one chimeric molecule of the aggregate is other than a chimeric molecule selected from the group consisting of: a B cell activating fusion protein comprising a B cell surface immunoglobulin binding domain and a signal peptide, wherein a catalytic product of the precursor is capable of inducing B cell mitogenesis; and a fusion protein comprising protein L and ompA, and wherein the self-coalescing element is represented by the formula:
B₁-X₁[Xⱼ]ₙX₂X₃X₄X₅[Xₖ]ₙX₆[Xₗ]ₙX₇X₈X₉-Z₁ (I)
[SEQ ID NO:1]
wherein: B₁, is absent or is a sequence of n amino acid residues wherein n is from about 1 to about 50 amino acid residues, wherein the sequence comprises the same or different amino acid residues selected from any amino acid residue;
X₁ is a hydrophobic, small, neutral or basic amino acid residue, or modified form thereof;
[Xⱼ]ₙ is a sequence of n amino acid residues wherein n is from 0 to 2 amino acid residues and wherein the sequence Xⱼ comprises the same or different amino acid residues selected from any amino acid residue;
X₂ is a hydrophobic, small or polar amino acid residue or modified form thereof;
X₃ is a hydrophobic, small or neutral/polar amino acid residue or modified form thereof;
X₄ is a hydrophobic or small amino acid residue or modified form thereof;
X₅ is a hydrophobic or small amino acid residue or modified form thereof;
[Xₖ] is a sequence of n amino acid residues wherein n is from 4 to 6 amino acid residues and wherein the sequence Xₖ comprises the same or different amino acid residues selected from a hydrophobic, small, polar or neutral amino acid residue or modified form thereof;
X₆ is a hydrophobic or small amino acid residue or modified form thereof;
[Xₗ]ₙ is a sequence of n amino acid residues wherein n is from 2 to 4 amino acid residues and wherein the sequence X₁ comprises the same or different amino acid residues selected from a hydrophobic, small or polar amino acid residue or modified form thereof;
X₇ is a hydrophobic, small, charged or neutral/polar amino acid residue or modified form thereof;
X₈ is a neutral/polar, charged, hydrophobic, or small amino acid residue or modified form thereof;
X₉ is optional and when present is selected from a small or charged amino acid residue or modified form thereof; and
Z₁, is absent or is a sequence of n amino acid residues wherein n is from about 1 to about 50 amino acid residues, wherein the sequence comprises the same or different amino acid residues selected from any amino acid residue.

52. The aggregate of claim 51, wherein when B₁ is present, it is a sequence of from about 1 to about 20 amino acid residues.

53. The aggregate of claim 51, wherein when B₁ is present, it is represented by the formula:
B₂J₁[Xᵢ]ₙ (II)
[SEQ ID NO:2]
wherein: B₂ is absent or is a sequence of n amino acid residues wherein n is from about ₁ to about 15 amino acid residues, wherein the sequence comprises the same or different amino acid residues selected from any amino acid residue, provided that J₁ is also present;
J₁ is absent or is a hydrophobic, charged, neutral/polar or small amino acid residue or modified form thereof, provided that [Xᵢ]ₙ is also present; and
[Xᵢ]ₙ is a sequence of n amino acid residues wherein n is from 2 to 5 amino acid residues and wherein the sequence Xᵢ comprises the same or different amino acid residues selected from any amino acid residue.

54. The aggregate of claim 53, wherein J₁ is a hydrophobic amino acid residue selected from Phe or Ile, or modified form thereof.

55. The aggregate of claim 53, wherein J₁ is a basic amino acid residue selected from His, Lys or Arg, or modified form thereof.

56. The aggregate of claim 53, wherein J₁ is Asn, or modified form thereof.

57. The aggregate of claim 53, wherein J₁ is a small amino acid residue selected from Ser or Thr, or modified form thereof.

58. The aggregate of claim 53, wherein [Xᵢ]ₙ is represented by the formula:
O₁O₂O₃O₄O₅ (III)
[SEQ ID NO:3]
wherein: at least two of O₁ to O₅ are present, in which:
O₁ is selected from a hydrophobic, charged, neutral/polar or small amino acid residue, or modified form thereof;
O₂ is selected from a small or basic amino acid residue, or modified form thereof;
O₃ is selected from a charged, neutral/polar, hydrophobic or small amino acid residue, or modified form thereof;
O₄ is selected from a charged, neutral/polar, hydrophobic or small amino acid residue, or modified form thereof; and
O₅ is selected from a charged, neutral/polar, hydrophobic or small amino acid residue, or modified form thereof.

59. The aggregate of claim 53, wherein [Xᵢ]ₙ is represented by the formula:
O₁O₂O₃O₄O₅ (III)
[SEQ ID NO:3]
wherein: at least two of O₁ to O₅ are present, in which:
O₁ is selected from Leu, Ile, Arg, Asn or Ala, or modified form thereof;
O₂ is selected from Thr or Lys, or modified form thereof;
O₃ is selected from Arg, Lys, Asn, Ile, Val, Leu or Ala, or modified form thereof;
O₄ is selected from Arg, Lys, Gln, Asn, Phe, Ile, Val, Leu, Ala, Gly, Ser, Thr, or modified. form thereof; and
O₅ is selected from Arg, Lys, Asn, Phe, Ile, Val, Leu, Ala, Gly, Ser, Thr, or modified form thereof.

60. The aggregate of claim 51, wherein X₁ is a hydrophobic amino acid residue selected from Leu, Met, Phe, Ile or Val, or modified form thereof.

61. The aggregate of claim 51, wherein X₁ is a small amino acid residue selected from Gly, Ala, Ser or Thr, or modified form thereof.

62. The aggregate of claim 51, wherein is selected from Cys, Lys or His, or modified form thereof.

63. The aggregate of claim 51, wherein [Xⱼ]ₙ is a single amino acid residue selected from Ala, Arg, Asn or Val, or modified form thereof.

64. The aggregate of claim 51, wherein [Xⱼ]ₙ is a sequence of two amino acid residues, wherein the first amino acid residue is selected from Lys, Asp, Leu, Asn, Ala, Val or Phe, or modified form thereof and wherein the second amino acid residue is selected from Ser, Ala, Lys, Gln, Asn or Leu, or modified form thereof.

65. The aggregate of claim 51, wherein X₂ is a hydrophobic amino acid residue selected from Val, Leu, Tyr, Ile or Phe, or modified form thereof.

66. The aggregate of claim 51, wherein X₂ is a small amino acid residue selected from Pro, Ala, Gly, Ser or Thr, or modified form thereof.

67. The aggregate of claim 51, wherein X₂ is selected from Asn or Arg, or modified form thereof.

68. The aggregate of claim 51, wherein X₃ is Ala or modified form thereof.

69. The aggregate of claim 51, wherein X₃ is a hydrophobic amino acid residue selected from Met, Leu, Val, Ile or Phe, or modified form thereof.

70. The aggregate of claim 51, wherein X₃ is Cys or modified form thereof.

71. The aggregate of claim 51, wherein X₄ is a hydrophobic amino acid residue selected from Val, Leu, Ile or Trp, or modified form thereof.

72. The aggregate of claim 51, wherein X₄ is a small amino acid residue selected from Ala, Gly, Ser or Thr, or modified form thereof.

73. The aggregate of claim 51, wherein X₅ is a small amino acid residue selected from Ala, Gly, Ser or Thr, or modified form thereof.

74. The aggregate of claim 51, wherein X₅ is a hydrophobic amino acid residue selected from Leu, Phe, Val, Ile, or modified form thereof.

75. The aggregate of claim 51, wherein [Xₖ]ₙ is represented by the formula:
B₃O₆O₇O₈O₉B₄ (IV)
[SEQ ID NO:4]
wherein: B₃ is selected from a small, hydrophobic or neutral/polar amino acid residue, or modified form thereof;
at least two of O₆ to O₉ are present, in which:
O₆ is selected from a small, hydrophobic or neutral/polar amino acid residue, or modified form thereof;
O₇ is selected from a small, hydrophobic or neutral/polar amino acid residue, or modified form thereof;
O₈ is selected from a small or hydrophobic amino acid residue, or modified form thereof; and
O₉ is selected from small, hydrophobic, basic or neutral/polar amino acid residue, or modified form thereof; and
B₄ is selected from a small or hydrophobic amino acid residue, or modified form thereof.

76. The aggregate of claim 75, wherein B₃ is selected from Pro, Ala, Gly, Ser, Thr, Val, Leu or Cys, or modified form thereof.

77. The aggregate of claim 75, wherein O₆ is selected from Ala, Gly, Ser, Thr, Val, Leu, Ile, Met or Cys, or modified form thereof.

78. The aggregate of claim 75, wherein O₇ is selected from Ala, Ser, Phe or Asn, or modified form thereof.

79. The aggregate of claim 75, wherein O₈ is selected from Thr, Ala, Ser, Ile, Leu, Val, Met, Phe, Tyr or Trp, or modified form thereof.

80. The aggregate of claim 75, wherein O₉ is selected from Pro, Ala, Gly, Ser, Thr, Ile, Leu, Val, Phe, His or Cys, or modified form thereof.

81. The aggregate of claim 75, wherein B₄ is selected from Ala, Ser, Thr, Ile, Val, Leu, Met, Tyr or Phe, or modified form thereof.

82. The aggregate of claim 51, wherein X₆ is a hydrophobic amino acid residue selected from Leu, Val, Met or Tyr, or modified form thereof.

83. The aggregate of claim 51, wherein X₆ is a small amino acid residue selected from Pro, Ala, Gly, Ser or Thr, or modified form thereof.

84. The aggregate of claim 51, wherein [Xₗ]ₙ is represented by the formula:
B₅O₁₀O₁₁O₁₂ (V)
[SEQ ID NO:5]
wherein: B₅ is selected from a small, hydrophobic or neutral/polar amino acid residue, or modified form thereof;
at least one of O₁₀ to O₁₂ are present, in which:
O₁₀ is selected from a small, hydrophobic or neutral/polar amino acid residue, or modified form thereof;
O₁₁ is a small amino acid residue; and
O₁₂ is selected from a small, hydrophobic or neutral/polar amino acid residue, or modified form thereof.

85. The aggregate of claim 84, wherein B₅ is selected from Pro, Ala, Gly, Ser, Thr, Ile, Leu, Val, Phe, Met or Gln, or modified form thereof.

86. The aggregate of claim 84, wherein O₁₀ is selected from Gly, Ala, Ser, Thr, Val, Leu, Met, Phe, Cys, Asn or Gln, or modified form thereof.

87. The aggregate of claim 84, wherein O₁₁ is Pro, or modified form thereof;.

88. The aggregate of claim 84, wherein O₁₂ is selected from Ala, Gly, Ser, Thr, Ile, Leu, Val, Tyr, Trp or Cys, or modified form thereof.

89. The aggregate of claim 51, wherein X₇ is a hydrophobic amino acid residue selected from Leu, Ile, Val or Met, or modified form thereof.

90. The aggregate of claim 51, wherein X₇ is a small amino acid residue selected from Pro, Ala, Gly, Ser or Thr, or modified form thereof.

91. The aggregate of claim 51, wherein X₇ is a charged amino acid residue, or modified form thereof.

92. The aggregate of claim 91, wherein X₇ is a basic amino acid residue selected from Asp or Arg, or modified form thereof.

93. The aggregate of claim 51, wherein X₇ is Asn, or modified form thereof.

94. The aggregate of claim 51, wherein X₈ is a neutral/polar amino acid residue selected from Gln, Asn or Cys, or modified form thereof.

95. The aggregate of claim 51, wherein X₈ is a charged amino acid residue, or modified form thereof.

96. The aggregate of claim 95, wherein X₈ is a basic amino acid residue selected from His or Glu, or modified form thereof.

97. The aggregate of claim 51, wherein X₈ is a hydrophobic amino acid residue selected from Val, Met or Trp, or modified form thereof.

98. The aggregate of claim 51, wherein X₈ is a small amino acid residue selected from Ala or Ser, or modified form thereof.

99. The aggregate of claim 51, wherein X₉ is a small amino acid residue selected from Ala, Gly, Ser or Thr, or modified form thereof.

100. The aggregate of claim 51, wherein X₉ is a charged amino acid residue, or modified form thereof.

101. The aggregate of claim 100, wherein X₉ is an acidic amino acid residue, or modified form thereof.

102. The aggregate of claim 100, wherein X₉ is Glu, or modified form thereof.

103. The aggregate of claim 51, wherein Z₁ is represented by the formula:
J₂J₃J₄Z₂ (VI)
[SEQ ID NO:6]
wherein: J₂ is a small amino acid residue, or modified form thereof;
J₃ is absent or is a charged amino acid residue, or modified form thereof, provided that J₂ is also present;
J₄ is absent or is a charged amino acid residue or modified form thereof, provided that J₃ is also present; and
Z₂ is absent or is a sequence of n amino acid residues wherein n is from about 1 to about 15 amino acid residues, wherein the sequence comprises the same or different amino acid residues selected from any amino acid residue, provided that J₄ is also present.

104. The aggregate of claim 103, wherein J₂ is Thr, or modified form thereof.

105. The aggregate of claim 103, wherein J₃ is a basic amino acid residue, or modified form thereof.

106. The aggregate of claim 103, wherein J₃ is Lys, or modified form thereof.

107. The aggregate of claim 103, wherein J₄ is a basic amino acid residue, or modified form thereof.

108. The aggregate of claim 103, wherein J₄ is Lys, or modified form thereof.

109. The aggregate of claim 51, wherein Z₁ comprise at least one charged amino acid residue, or modified form thereof.

110. The aggregate of claim 51, wherein Z₁ comprise at least one basic amino acid residue, or modified form thereof.

111. The aggregate of claim 103, wherein Z₂ comprise at least one charged amino acid residue, or modified form thereof.

112. The aggregate of claim 103, wherein Z₂ comprise at least one basic amino acid residue, or modified form thereof.

113. An isolated or purified higher order aggregate comprising a plurality of chimeric molecules, wherein each chimeric molecule comprises at least one self-coalescing element, which is obtainable or derivable from a membrane translocating sequence or variant thereof, and which is fused, linked or otherwise associated with a molecule of interest, and wherein the or each self-coalescing element is capable of causing an individual chimeric molecule to coalesce with other chimeric molecules into higher order aggregates under conditions favorable to aggregation, wherein at least one chimeric molecule of the aggregate is other than a chimeric molecule selected from the group consisting of: a B cell activating fusion protein comprising a B cell surface immunoglobulin binding domain and a signal peptide, wherein a catalytic product of the precursor is capable of inducing B cell mitogenesis; and a fusion protein comprising protein L and ompA, and wherein the self-coalescing element is represented by the formula:
B₂J₁[Xᵢ]ₙXₗ[Xⱼ]ₙX₂X₃X₄X₅[Xₖ]ₙX₆[Xₗ]ₙX₇X₈X₉Z₁ (VII)
[SEQ ID NO:7]
wherein: B₂, J₁ and [Xᵢ]ₙ, are as defined in claim 53; and [Xⱼ]ₙ, [Xk]ₙ, [Xₗ]ₙ, X₁₋₉ and Z₁ are as defined in claim 51.

114. An isolated or purified higher order aggregate comprising a plurality of chimeric molecules, wherein each chimeric molecule comprises at least one self-coalescing element, which is obtainable or derivable from a membrane translocating sequence or variant thereof, and which is fused, linked or otherwise associated with a molecule of interest, and wherein the or each self-coalescing element is capable of causing an individual chimeric molecule to coalesce with other chimeric molecules into higher order aggregates under conditions favorable to aggregation, wherein at least one chimeric molecule of the aggregate is other than a chimeric molecule selected from the group consisting of: a B cell activating fusion protein comprising a B cell surface immunoglobulin binding domain and a signal peptide, wherein a catalytic product of the precursor is capable of inducing B cell mitogenesis; and a fusion protein comprising protein L and ompA, and wherein the self-coalescing element is represented by the formula:
B₁X₁X₂X₃X₄X₅[Xₘ]ₙX₆X₇X₈X₉X₁₀X₁₁X₁₂X_{13 l X}₁₄X₁₅X₁₆-Z₁ (VIII)
[SEQ ID NO:8]
wherein: B₁ is absent or is a sequence of n amino acid residues wherein n is from about 1 to about 5 amino acid residues, wherein the sequence comprises the same or different amino acids selected from any amino acid residue;
X₁ is a hydrophobic amino acid residue or modified form thereof;
X₂ is a small amino acid residue or modified form thereof;
X₃ is a hydrophobic amino acid residue or modified form thereof;
X₄ is selected from a hydrophobic or small amino acid residue or modified form thereof;
X₅ is a hydrophobic amino acid residue or modified form thereof; and
[Xₘ]ₙ is a sequence of n amino acid residues wherein n is from 0 to 2 amino acid residues and wherein the sequence Xₘ comprises the same or different amino acid residues selected from a hydrophobic or a small amino acid residue or modified form thereof;
X₆ is a small or hydrophobic amino acid residue or modified form thereof;
X₇ is a hydrophobic or small amino acid residue or modified form thereof;
X₈ is a hydrophobic or small amino acid residue or modified form thereof;
X₉ is a hydrophobic or small amino acid residue or modified form thereof;
X₁₀ is a hydrophobic, small or neutral/polar amino acid residue or modified form thereof;
X₁₁ is a small, hydrophobic or neutral/polar amino acid residue or modified form thereof;
X₁₂ is a small amino acid residue or modified form thereof;
X₁₃ is a hydrophobic or small amino acid residue or modified form thereof;
X₁₄ is a small amino acid residue or modified form thereof;
X₁₅ is a neutral/polar, acidic or hydrophobic amino acid residue or modified form thereof;
X₁₆ is a small amino acid residue or modified form thereof; and
Z₁ is absent or is a sequence of n amino acid residues wherein n is from about 1 to about 20 amino acid residues wherein the sequence comprises the same or different amino acid residues selected from any amino acid residue.

115. The aggregate of claim 114, wherein when B₁ is present, it is represented by the formula:
J₁J₂J₃J₄J₅ (IX)
[SEQ ID NO:9]
wherein: J₁ is absent or is a hydrophobic amino acid residue, or modified form thereof, provided that J₂ is also present;
J₂ is absent or is a charged amino acid residue, or modified form thereof, provided that J₃ is also present;
J₃ is absent or is a charged amino acid residue, or modified form thereof, provided that J₄ is also present;
J₄ is absent or is selected from a small, charged or neutral/polar amino acid residue, or modified form thereof, provided that J₅ is also present; and
J₅ is absent or is selected from a small or hydrophobic amino acid residue, or modified form thereof.

116. The aggregate of claim 115, wherein J₁ is Met, or modified form thereof.

117. The aggregate of claim 115, wherein J₂ is a basic amino acid residue, or modified form thereof.

118. The aggregate of claim 115, wherein J₂ is Lys, or modified form thereof.

119. The aggregate of claim 115, wherein J₃ is a basic amino acid residue, or modified form thereof.

120. The aggregate of claim 115, wherein J₃ is selected from Lys or Arg, or modified form thereof.

121. The aggregate of claim 115, wherein J₄ is Thr, or modified form thereof.

122. The aggregate of claim 115, wherein J₄ is a charged amino acid residue, or modified form thereof.

123. The aggregate of claim 115, wherein J₄ is a basic amino acid residue, or modified form thereof.

124. The aggregate of claim 115, wherein J₄ is selected from Lys or Arg, or modified form thereof.

125. The aggregate of claim 115, wherein J₄ is Gln, or modified form thereof.

126. The aggregate of claim 115, wherein J₅ is a small amino acid residue selected from Ala or Thr, or modified form thereof.

127. The aggregate of claim 115, wherein J₅ is Leu, or modified form thereof.

128. The aggregate of claim 114, wherein X₁ is selected from Ile, Val or Leu, or modified form thereof.

129. The aggregate of claim 114, wherein X₂ is selected from Thr, Gly, or Ala, or modified form thereof.

130. The aggregate of claim 114, wherein X₃ is selected from Ile or Leu, or modified form thereof.

131. The aggregate of claim 114, wherein X₄ is a hydrophobic amino acid residue selected from Val or Trp, or modified form thereof.

132. The aggregate of claim 114, wherein X₄ is a small amino acid residue selected from Ala, Ser or Thr, or modified form thereof.

133. The aggregate of claim 114, wherein X₅ is selected from Ile, Phe or Val, or modified form thereof.

134. The aggregate of claim 114, wherein [Xₗ]ₙ, is represented by the formula:
J₆J₇ (X)
[SEQ ID NO:10]
wherein: at least one of J₆ and J₇ are present, in which
J₆ is selected from a hydrophobic or small amino acid residue, or modified form thereof; and
J₇ is selected from a small or hydrophobic amino acid residue, or modified form thereof.

135. The aggregate of claim 134, wherein J₆ is Leu or Gly, or modified form thereof.

136. The aggregate of claim 134, wherein J₇ is Ser or Leu, or modified form thereof.

137. The aggregate of claim 114, wherein X₆ is Ala, or modified form thereof.

138. The aggregate of claim 114, wherein X₆ is a hydrophobic amino acid residue selected from Val or Leu, or modified form thereof.

139. The aggregate of claim 114, wherein X₇ is a small amino acid residue selected from Ala, Gly or Thr, or modified form thereof.

140. The aggregate of claim i14, wherein X₇ is Leu, or modified form thereof.

141. The aggregate of claim 114, wherein X₈ is a hydrophobic amino acid residue selected from Leu or Val, or modified form thereof.

142. The aggregate of claim 114, wherein X₈ is a small amino acid residue selected from Ala or Ser, or modified form thereof.

143. The aggregate of claim 114, wherein X₉ is a hydrophobic amino acid residue selected from Val or Leu, or modified form thereof.

144. The aggregate of claim 114, wherein X₉ is a small amino acid residue selected from Ala or Gly, or modified form.

145. The aggregate of claim 114, wherein X₁₀ is Gln or modified form thereof.

146. The aggregate of claim 114, wherein X₁₀ is a hydrophobic amino acid residue selected from Ile, Val or Phe, or modified form.

147. The aggregate of claim 114, wherein X₁₁ is a small amino acid residue selected from Pro, Ala or Thr or modified form thereof.

148. The aggregate of claim 114, wherein X₁₁ is Phe or modified form thereof.

149. The aggregate of claim 114, wherein X₁₁ is Gln, or modified form thereof.

150. The aggregate of claim 114, wherein X₁₂ is a small amino acid residue selected from Ala, Ser or Thr, or modified form thereof.

151. The aggregate of claim 114, wherein X₁₃ is a hydrophobic amino acid residue selected from Val, Ile or Met, or modified form thereof.

152. The aggregate of claim 114, wherein X₁₃ is Ala or modified form thereof.

153. The aggregate of claim 114, wherein X₁₄ is selected from Pro or Ala, or modified form thereof.

154. The aggregate of claim 114, wherein X₁₅ is Gln, or modified form thereof.

155. The aggregate of claim 114, wherein X₁₅ is Asp, or modified form thereof.

156. The aggregate of claim 114, wherein X₁₅ is Leu, or modified form thereof.

157. The aggregate of claim 114, wherein X₁₆ is Ala, or modified form thereof.

158. The aggregate of claim 114, wherein Z₁ is represented by the formula:
J₈J₉J₁₀ (XI)
[SEQ ID NO:11]
wherein: J₈ is a small amino acid residue, or modified form thereof;
J₉ is absent or is a charged amino acid residue, or modified form thereof, provided that J₈ is also present; and
J₁₀ is absent or is a charged amino acid residue, or modified form thereof, provided that J₉ is also present.

159. The aggregate of claim 158, wherein J₈ is Thr, or modified form thereof.

160. The aggregate of claim 158, wherein J₉ is a basic amino acid residue, or modified form thereof.

161. The aggregate of claim 158, wherein J₉ is Lys, or modified form thereof.

162. The aggregate of claim 158, wherein J₁₀ is a basic amino acid residue, or modified form thereof.

163. The aggregate of claim 158, wherein J₁₀ is Lys, or modified form thereof.

164. An isolated or purified chimeric molecule comprising a self-coalescing element that is obtainable or derivable from a membrane translocating sequence or variant thereof, which is fused attached or otherwise associated with a molecule of interest.

165. The chimeric molecule of claim 164, further comprising a linker or spacer molecule which spaces the molecule of interest from the self-coalescing element sufficiently so as to promote the proper folding of the molecule of interest.

166. The chimeric molecule of claim 165, wherein the linker or spacer molecule spaces the molecule of interest from the self-coalescing element sufficiently such that the molecule of interest retains a desired activity when the chimeric molecule forms aggregates with other chimeric molecules.

167. The chimeric molecule of claim 165, wherein the linker or spacer molecule prevents or reduces any intracellular cleavage of the self-coalescing element from the molecule of interest.

168. The chimeric molecule of claim 165, wherein the linker or spacer molecule is from about 1 to about 100 atoms in length.

169. The chimeric molecule of claim 165, wherein the linker or spacer molecule is from about 1 to about 50 amino acid residues in length.

170. The chimeric molecule of claim 165, wherein the linker or spacer molecule is an amino acid sequence selected from the group consisting of SEQ ID NO:167, 169, 171, 173, 175, 179, 181 and 183.

171. A polynucleotide comprising a nucleotide sequence that encodes the chimeric molecule of claim 164.

172. A vector that comprises a polynucleotide comprising a nucleotide sequence that encodes the chimeric molecule of any one of claims 164 to 170, operably linked to a regulatory element.

173. A host cell containing a vector that comprises a polynucleotide comprising a nucleotide sequence that encodes the chimeric molecule of claim 164, operably linked to a regulatory element.

174. A genetically modified animal having cells that comprise a polynucleotide comprising a nucleotide sequence that encodes the chimeric molecule of claim 164, operably linked to a regulatory element.

175. A method for enhancing the activity of a molecule of interest, or for combining distinct activities of different molecules of interest, the method comprising linking, fusing or otherwise associating individual molecules of interest with a self-coalescing element that is obtainable or derivable from a membrane translocating sequence or variant thereof, wherein a chimeric molecule thus produced is caused by the self-coalescing element to coalesce with other chimeric molecules into a higher molecular weight aggregate.

176. A pharmaceutical or veterinary composition comprising the aggregate of any one of claims 1, 51, 113 and 114, and a pharmaceutically-acceptable carrier.

177. The pharmaceutical or veterinary composition of claim 176, wherein the composition is immunopotentiating.

178. The composition of claim 177, additionally comprising an adjuvant.

179. A method of treating or preventing cancer or an infection in a patient, comprising administering an effective amount of a composition according to claim 176.
